(19) United States
(12) Patent Application Publication
(10) Pub. No.: US 2023/0031576 A1
(43) Pub. Date: Feb. 2, 2023

(54) RADIOLABELLED TARGETING LIGANDS

(71) Applicants: The University of Queensland, St Lucia, Queensland (AU); Clarity Pharmaceuticals Ltd, Eveleigh, New South Wales (AU)

(72) Inventors: Kristofer James THURECHT, St Lucia, Queensland (AU); Vanessa SOH YING YI, St Lucia, Queensland (AU); Idriss BLAKEY, St Lucia, Queensland (AU); Muneer Ahamed Syed MUSTHAKAHMED, St Lucia, Queensland (AU); Matthew HARRIS, Eveleigh, New South Wales (AU); Ellen VAN DAM, Eveleigh, New South Wales (AU)

(21) Appl. No.: 17/775,150

(22) PCT Filed: Nov. 6, 2020
(86) PCT No.: PCT/AU2020/051209

§ 371 Date: May 6, 2022

(30) Foreign Application Priority Data
Nov. 8, 2019 (AU) 2019904218

(51) Int. Cl. A61K 51/04

(52) U.S. Cl. CPC A61K 51/0497; A61K 51/0482

(57) ABSTRACT

The present invention relates to compounds that are useful as radioimaging agents and radiopharmaceuticals. The compounds may be coordinated with a radionuclide and may be useful in diagnostic imaging and radiotherapy. The invention also relates to methods of prognosis and therapy utilising the non-coordinated and radiolabelled compounds of the invention.

### FIELD

The present invention relates to compounds that are useful as radioimaging agents and radiopharmaceuticals. The compounds may be coordinated with a radionuclide and may be useful in diagnostic imaging and radiotherapy. The invention also relates to methods of prognosis and therapy utilising the non-coordinated and radiolabelled compounds of the invention.

### BACKGROUND

Radiolabelled compounds may be used as radioimaging agents or radiopharmaceuticals. In order for such compounds to be used in radioimaging or as a radiopharmaceutical, the compound must be able to contain a radionuclide and also have the requisite stability, compatibility and other physical properties.

Radiolabelled compounds are often used for the imaging or treatment of tumours and related cancers. Compounds that are suitable for such use contain a fragment that is capable of binding to a given receptor or target site that is characteristic of a particular tumour or cancer. The compound also contains a radionuclide that has the requisite decay properties, such that the products of decay allow for the eventual imaging and treatment of the tumour or cancer. The exact nature of the radiolabelled compound depends on the site to be targeted.

The compound must be able to coordinate the radionuclide such that dissociation of the radionuclide is minimised. Dissociation of the radionuclide, especially in vivo after administration, is unwanted, as circulation of the free radionuclide may lead to unwanted damage to other tissues and also reduced efficacy of the radiolabelled compound for radioimaging or therapy. Since the radionuclide naturally undergoes radioactive decay, the decay products may lead to decomposition of the compound coordinating the radionuclide. Known as radiolysis, this phenomenon may result in the unwanted dissociation of the radionuclide thereby affecting the overall efficacy of the administered compound and producing unwanted effects.

As the radiolabelled compounds contain at least a fragment intended to bind the target site and a fragment capable of coordinating or chelating the radionuclide. These multicomponent compounds rely on synthetic routes that enable the requisite fragments to be assembled. Since a particular fragment may contain one or more reactive functional groups, the synthetic route must be selective for the desired functional groups and minimise any unwanted side reactions from occurring. As the nature of the fragment that is intended to bind the target site strongly depends on the target site itself, the synthetic route that provides access to the compound must be compatible with any existing functionalities. The coordinated compound containing the radionuclide is typically accessed by exposing the free compound to the radionuclide, however these reactions often provide the radiolabelled compound in low yields.

There remains a need for new radiolabelled compounds that can be used in radioimaging and radiotherapy. The compounds should be sufficiently stable, have the requisite binding selectivity and be accessible in sufficient yields.

### SUMMARY OF THE INVENTION

The present invention relates to novel compounds that bind to the chemokine receptor 4 (CXCR4). The present inventors have found that the use of a particular macrocyclic ligand (sarcophagine) which is bound to a particular cyclic polypeptide via certain linkers provides compounds that effectively bind to the CXCR4 receptor. When complexed with a radioisotope, the ligand-radioisotope complex provides beneficial radioimaging and radiotherapeutic properties.

In a first aspect, the present invention provides a compound of Formula (I) or a salt, complex, isomer, solvate or prodrug thereof:

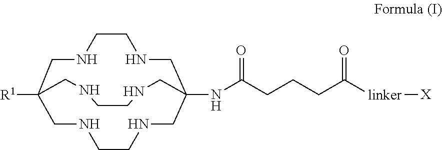

wherein:

R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;

X is

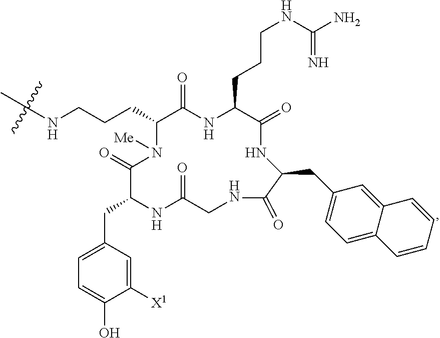

wherein X¹ is H or iodo; and

the linker is absent or selected from:

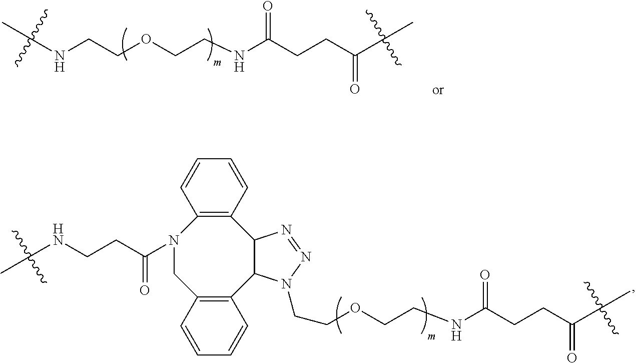

wherein m is independently an integer from 1 to 10.

In an embodiment, the linker is absent and the compound of Formula (I) has the following structure:

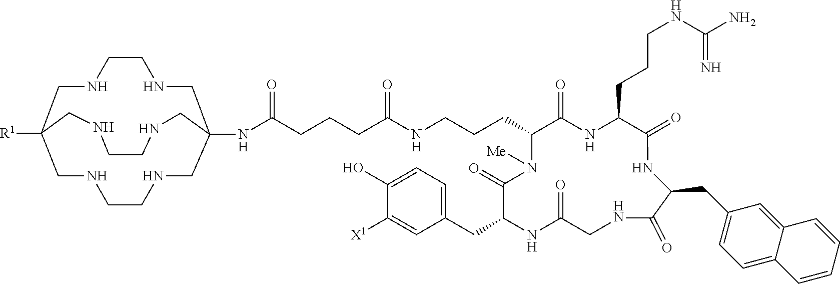

wherein R¹ and X¹ have the definitions above.

In an embodiment, the compound of Formula (I) has the following structure:

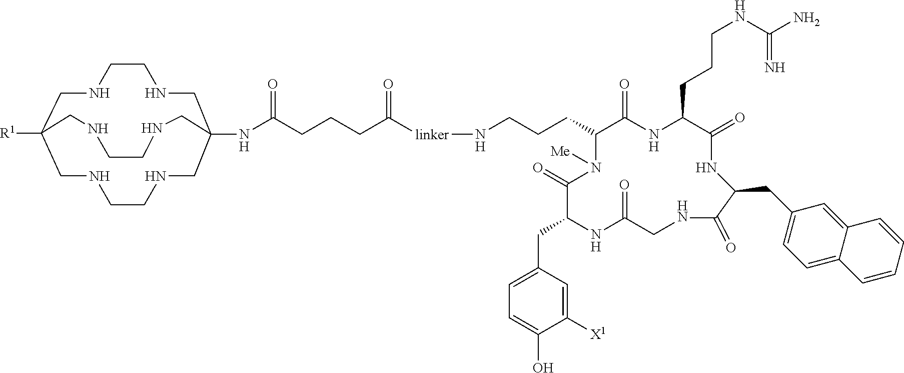

wherein the linker is selected from:

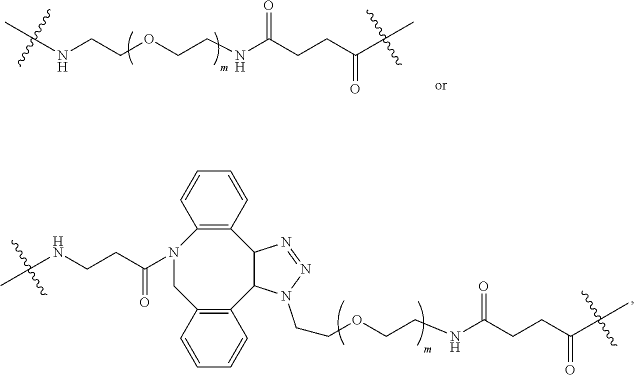

and wherein R¹, X¹ and m have the definitions above.

In another embodiment, the compound of Formula (I) has the following structure:

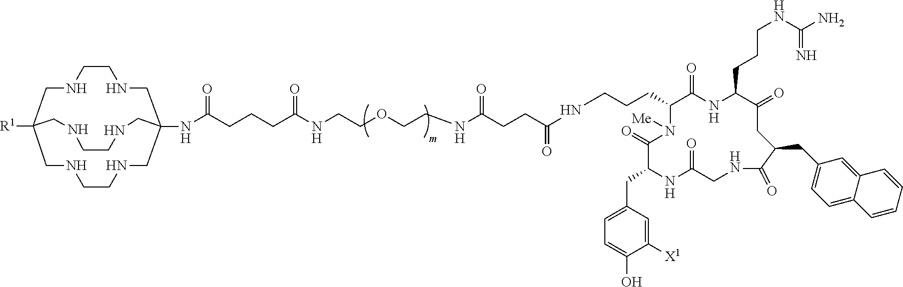

wherein R¹, X¹ and m have the definitions above.

In another embodiment, the compound of Formula (I) has the following structure:

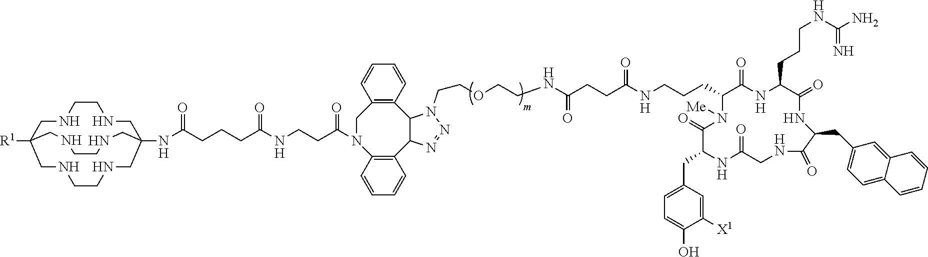

wherein R¹, X¹ and m have the definitions above.

In a second aspect, the present invention provides a compound of Formula (II) or salt, complex, isomer, solvate or prodrug thereof:

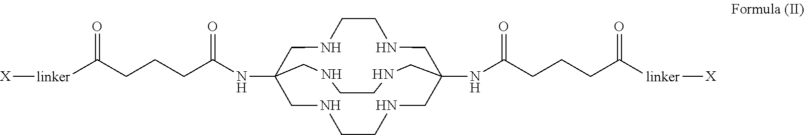

wherein:

X is

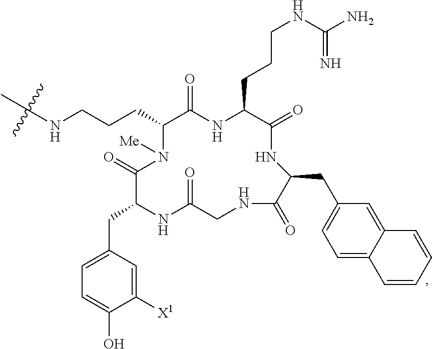

wherein X¹ is H or iodo; and

the linker is absent or selected from:

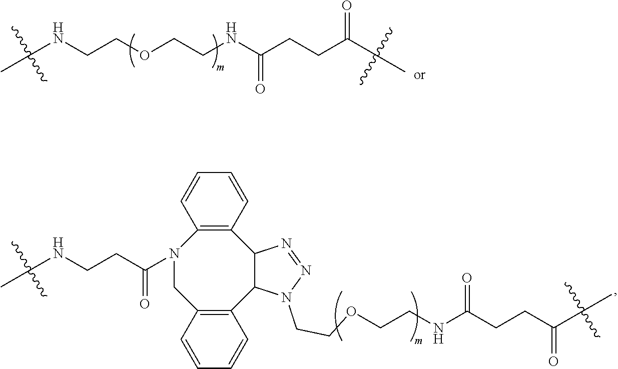

wherein m is independently an integer from 1 to 10.

In an embodiment, the linker is absent and the compound of Formula (II) has the following structure:

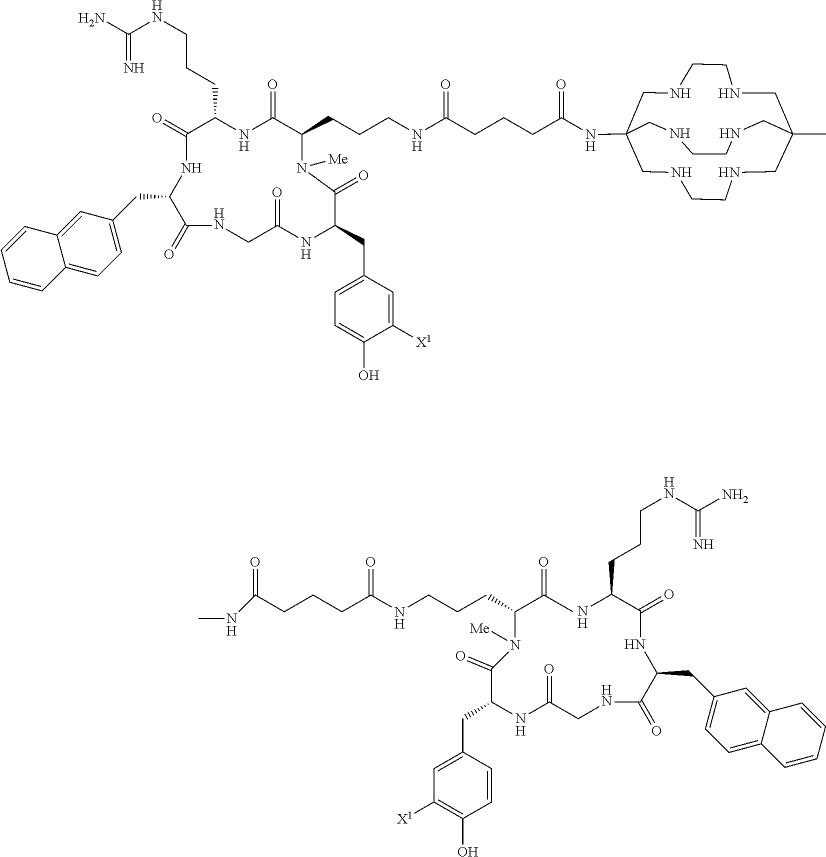

wherein X¹ has the definition above.

In an embodiment, the compound of Formula (II) has the following structure:

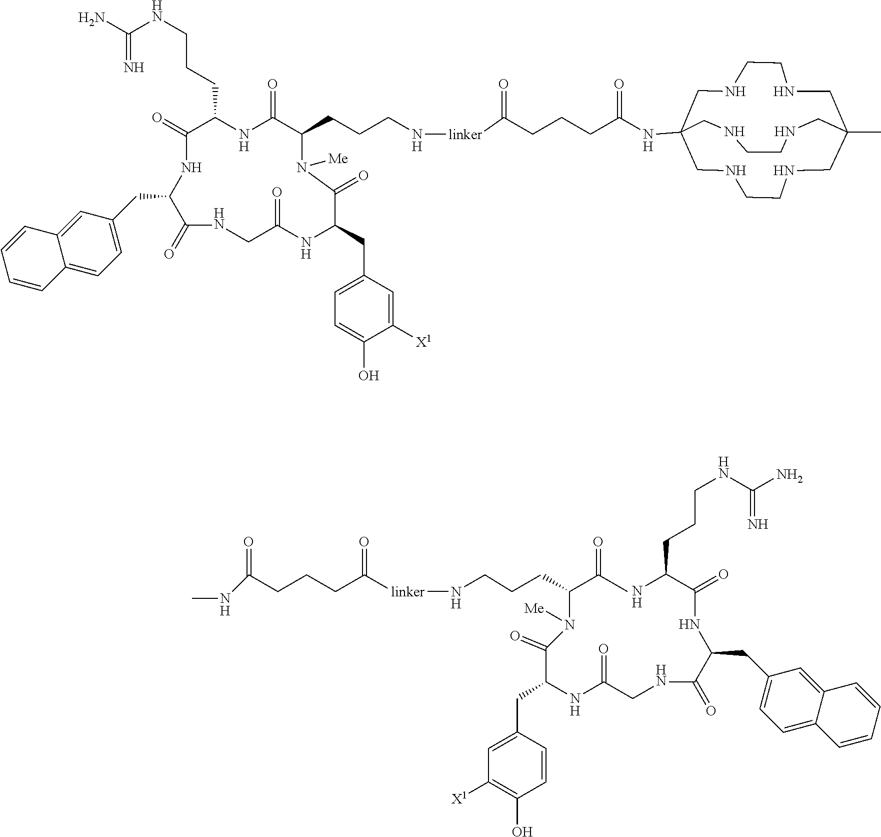

wherein the linker is selected from:

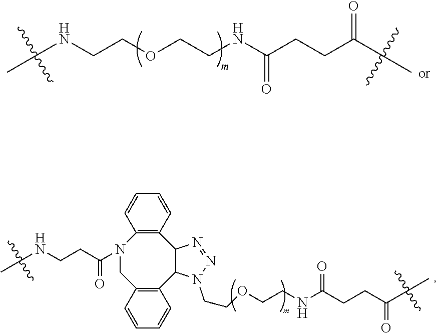

wherein X¹ and m have the definitions above.

In another embodiment, the compound of Formula (II) has the following structure:

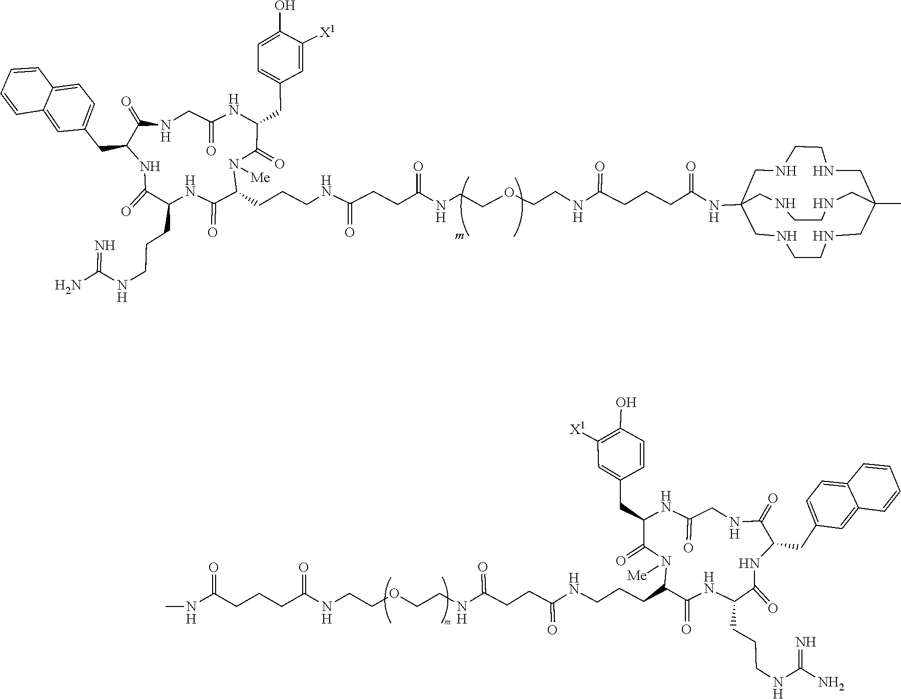

wherein X¹ and m have the definitions above.

In another embodiment, the compound of Formula (II) has the following structure:

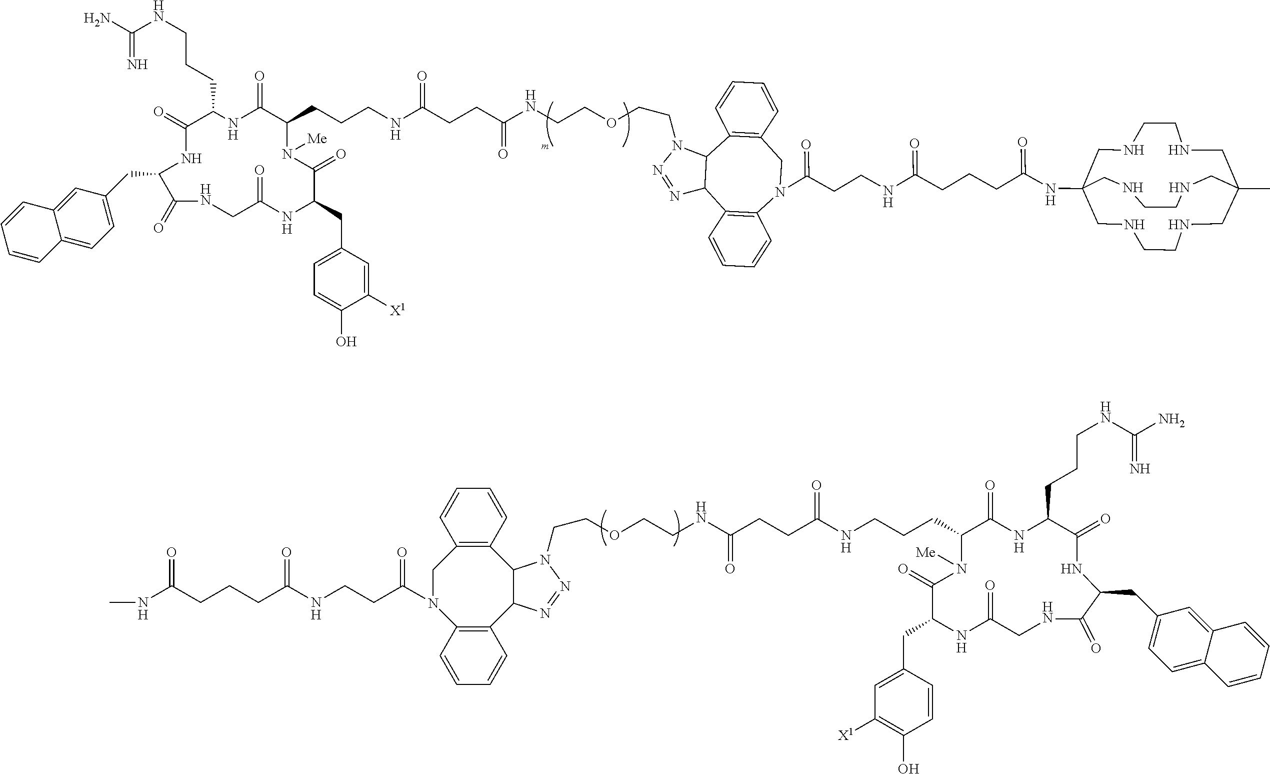

wherein X¹ and m have the definitions above.

In a third aspect, the present invention provides a compound of Formula (III) or a salt, complex, isomer, solvate or prodrug thereof:

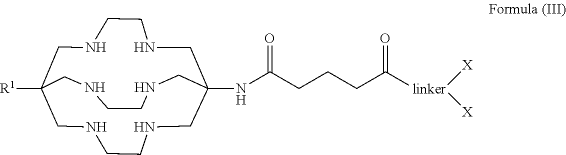

wherein:

R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;

X is

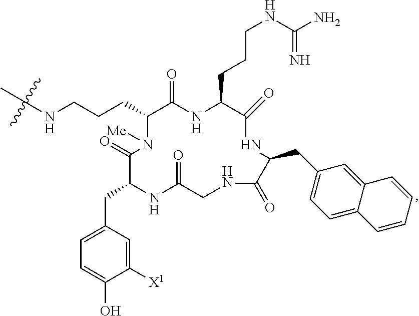

wherein X¹ is H or iodo; and

the linker is selected from:

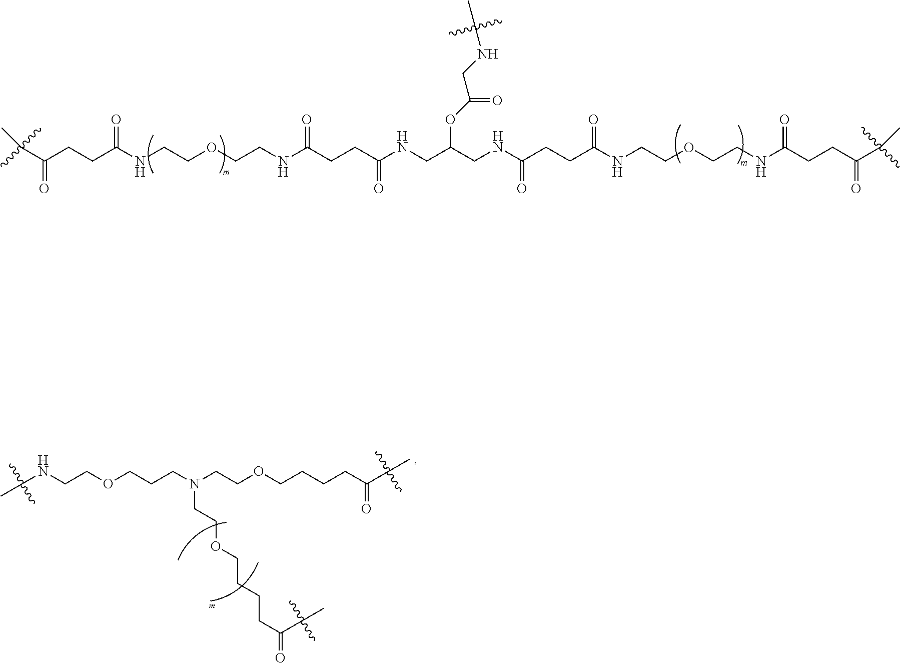

wherein mis independently an integer from 1 to 10.

In an embodiment, the compound of Formula (III) has the following structure:

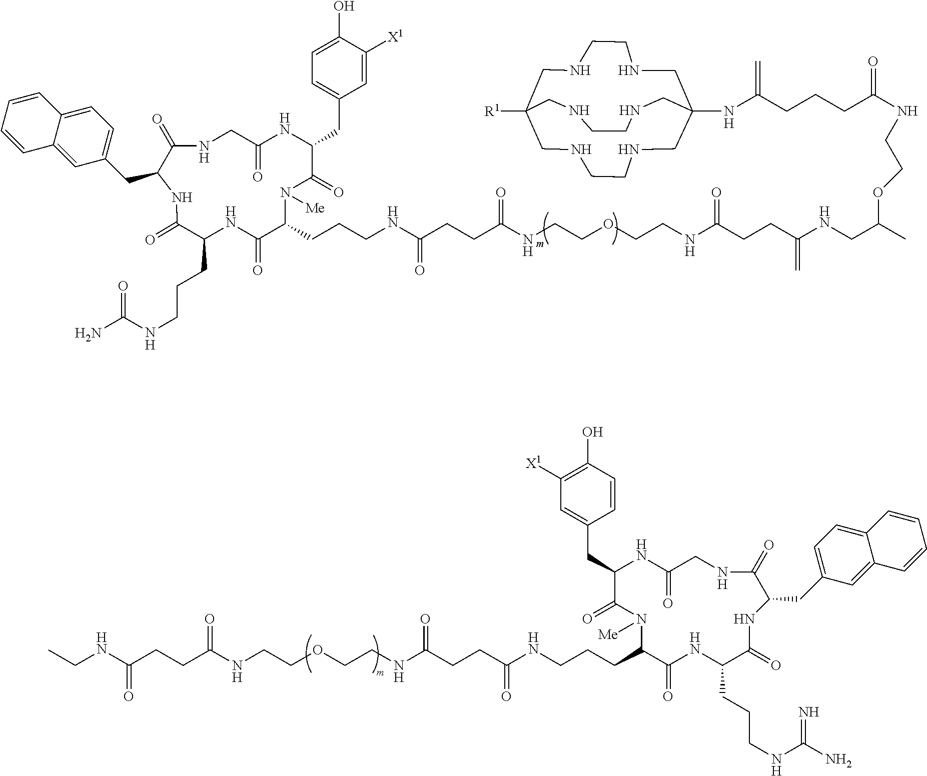

wherein R¹, X¹ and m have the definitions above.

In an embodiment, the compound of Formula (III) has the following structure:

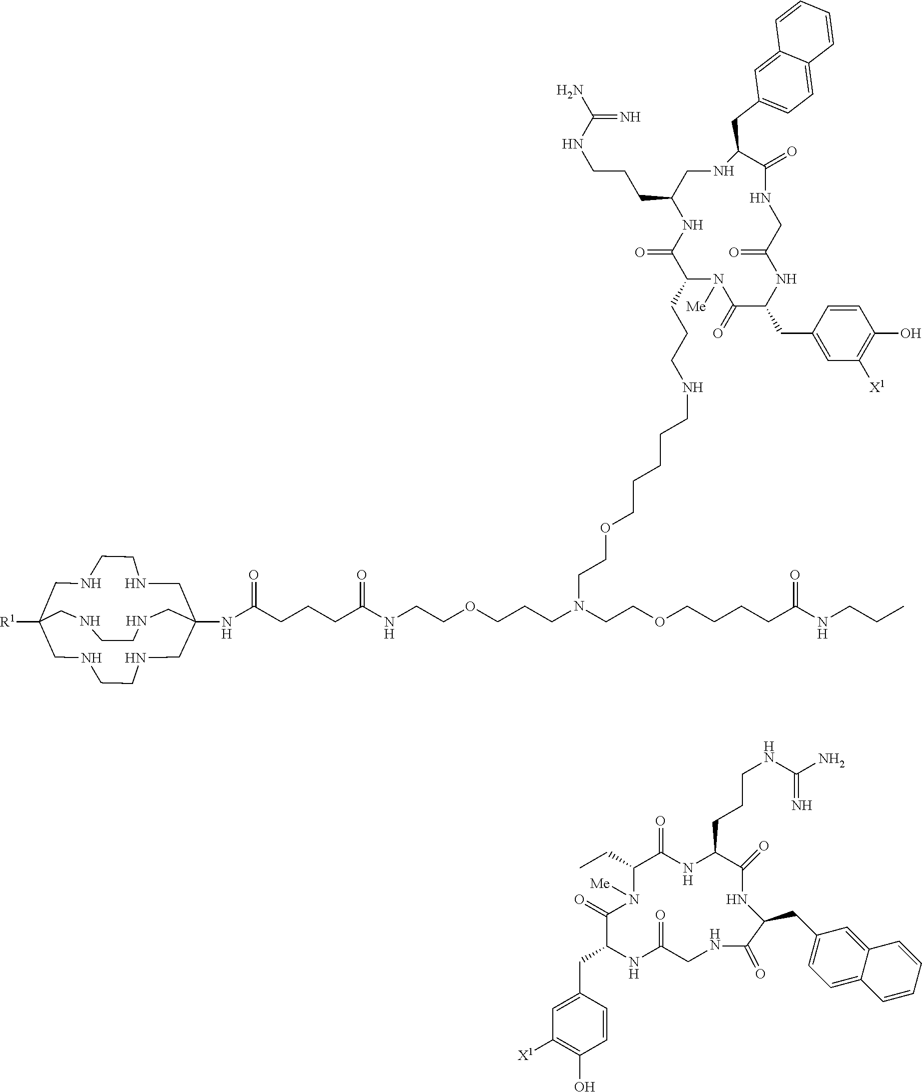

wherein R¹, X¹ and m have the definitions above.

In a fourth aspect, the present invention provides a compound of Formula (IV) or salt, complex, isomer, solvate or prodrug thereof:

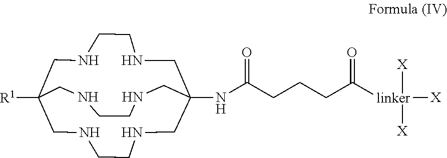

wherein:

R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;

X is

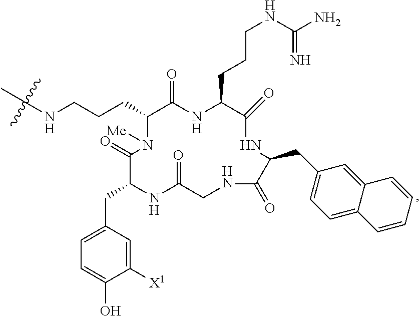

wherein X¹ is H or iodo; and

the linker is selected from:

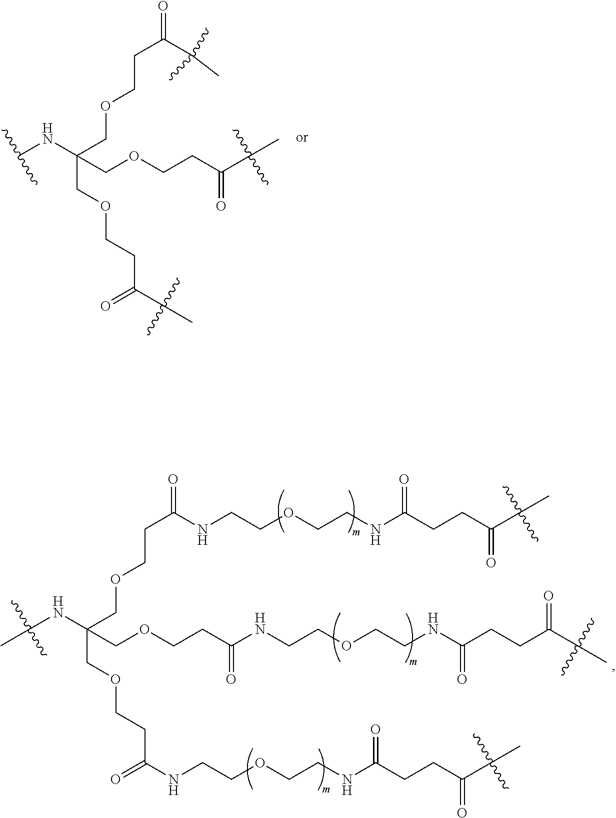

wherein m is an integer from 1 to 10.

In an embodiment, the compound of Formula (IV) has the following structure:

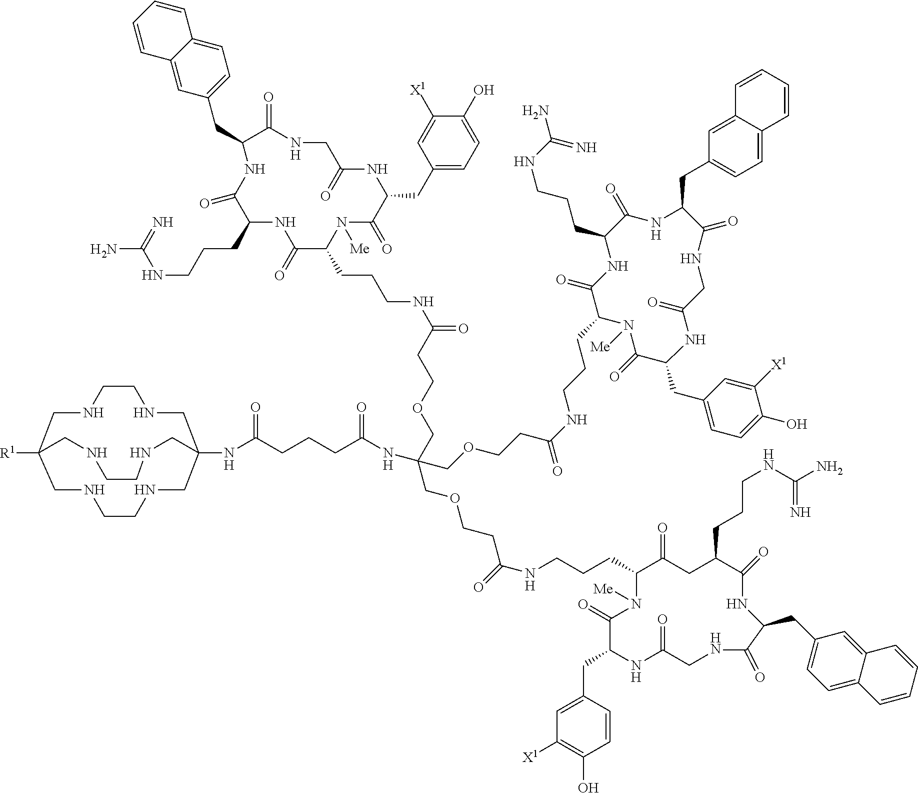

wherein R¹, X¹ have the definitions above.

In another embodiment, the compound of Formula (IV) has the following structure:

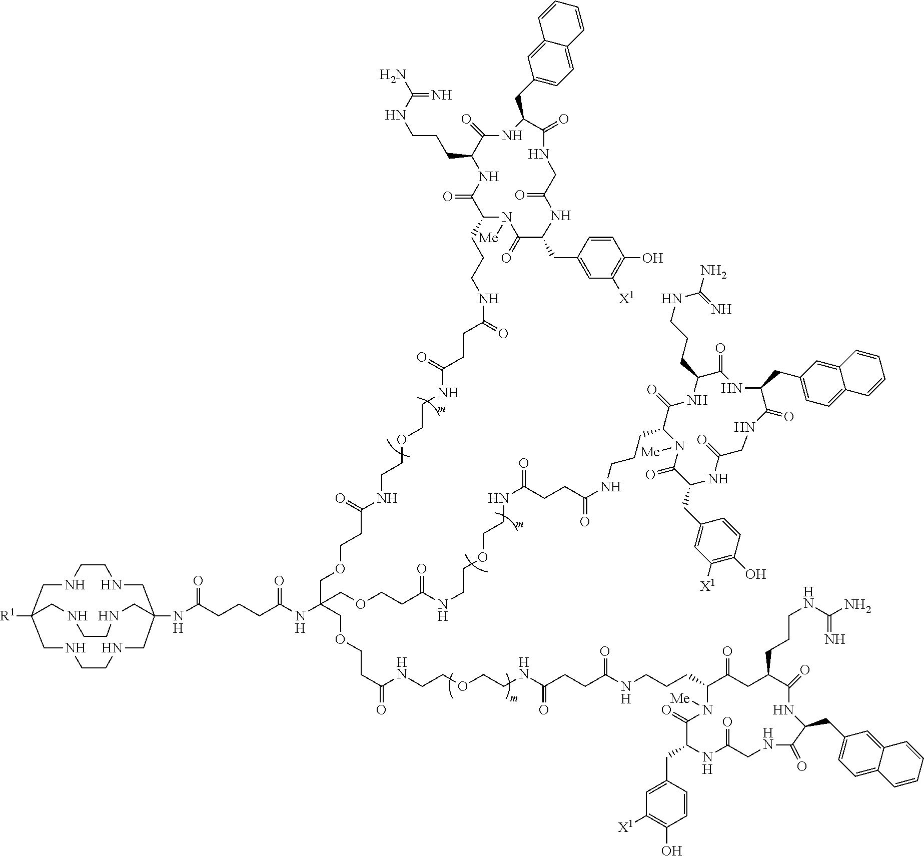

wherein R¹, X¹ and m have the definitions above.

In an embodiment, the compound of Formula (I), (II), (III) or (IV) is complexed with a metal. In another embodiment, the compound of Formula (I), (II), (III) or (IV) is complexed with a metal that is a radioisotope. In another embodiment, the radioisotope is a copper (Cu) radioisotope.

In a fifth aspect, the present invention provides a composition comprising a compound according to the first to fourth aspects, or a salt thereof, and one or more pharmaceutically acceptable excipients.

In a sixth aspect, the present invention provides a method for radioimaging a subject, the method comprising administering to the subject a compound according to the first to fourth aspects, or a salt thereof, or a composition according to the fifth aspect.

In a seventh aspect, the present invention provides a method for treating or preventing a condition in a subject, the method comprising administering to the subject a compound according to the first to fourth aspects, or a salt thereof, or a composition according to the fifth aspect.

In an embodiment, the condition is a cancer or a tumour.

In an eighth aspect, the present invention provides use of a compound according to the first to fourth aspects or a salt thereof in the manufacture of a medicament for treating or preventing a condition.

In an embodiment, the condition is a cancer or a tumour.

### BRIEF DESCRIPTION OF THE FIGURES

FIG. 1. ¹H NMR spectrum of compound 8.

FIG. 2. ¹³C NMR spectrum of compound 8.

FIG. 3. ¹⁹F NMR spectrum for compound 8.

FIG. 4. ESI−MS spectra for compound 8.

FIG. 5. ¹H NMR spectrum for compound 9.

FIG. 6. ¹³C NMR spectrum for compound 9.

FIG. 7. MALDI-TOF spectrum for compound 9.

FIG. 8. ESI−MS spectra for compound 11.

FIG. 9. ESI−MS spectrum for compound 12.

FIG. 10. ESI−MS spectra for compound 13.

FIG. 11. ESI−MS spectrum for compound 14.

FIG. 12. Analytical HPLC chromatogram for compound 15.

FIG. 13. ESI−MS spectra for compound 15.

FIG. 14. Analytical HPLC chromatogram for compound 16.

FIG. 15. ESI−MS spectra for compound 16.

FIG. 16. Analytical HPLC chromatogram for compound 17.

FIG. 17. ESI−MS spectra for compound 17.

FIG. 18. HPLC chromatogram for compound 18.

FIG. 19. ESI−MS spectrum for compound 18.

FIG. 20. HPLC chromatogram for compound 19.

FIG. 21. ESI−MS spectra for compound 19.

FIG. 22. HPLC chromatogram for compound 20.

FIG. 23. ESI−MS spectra for compound 20.

FIG. 24. Tabulated results for radiolabelled peptides for Example 7 animal experiments

FIG. 25. Images of rat dosing studies of Example 7 over time (22 hrs).

FIG. 26. Graphs of dosage studies of Example 7.

FIG. 27. Bio distribution graph of Example 7 dosage studies at 22 hr p.i. comparison between both tracers (Organs were weighed and activity counted using gamma counter to calculate % ID/g).

FIG. 28. Graph depicting Example 7 cell uptake studies over time.

FIG. 29. HPLC trace of (t-BOC)₄₋₅ BisCOSar-peptide of Example 8.

FIG. 30. LCMS trace of branched PEG peptide analogue of Example 10.

FIG. 31. ESI−MS of (t-BOC)₄₋₅ BisCOSar-peptide of Example 8.

FIG. 32. HPLC trace of branched PEG peptides of Example 11.

FIG. 33. EIS-MS trace of branched PEG peptides of Example 11.

FIG. 34. LCMS trace of crude bifunctional compound of Example 11.

FIG. 35. Table showing radiochemical yield and purity over time for compounds of Example 13.

FIG. 36a. Graph depicting specific binding based on % Applied dose over time for compounds disclosed in Example 13.

FIG. 36b. Graph depicting specific internalization based on % Applied dose over time for compounds disclosed in Example 13.

FIG. 37a. Graph depicting radiolabel stability of [64Cu]SAR-PEG3-pentixather of Example 14.

FIG. 37b. Graph depicting radiolabel stability of [64Cu]SAR-bis-pentixather of Example 14.

FIG. 38. Table showing LogD values for the compounds of Example 16.

FIG. 39. HPLC trace of HAS trimer stability of Example 17.

FIG. 40a. Graph depicting binding over time of trimer of Example 17.

FIG. 40b. Graph depicting internalisation over time of trimer of Example 17.

FIG. 41a. Graph depicting comparison cellular binding over time of trimer vs mono and bis pentixanther (Example 18).

FIG. 41b. Graph depicting comparison internalisation over time of trimer vs mono and bis pentixanther (Example 18).

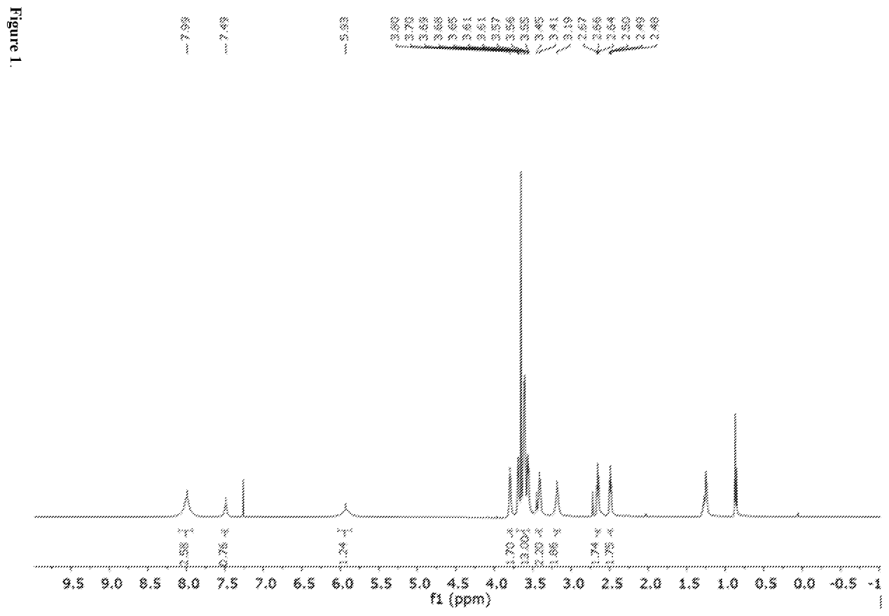

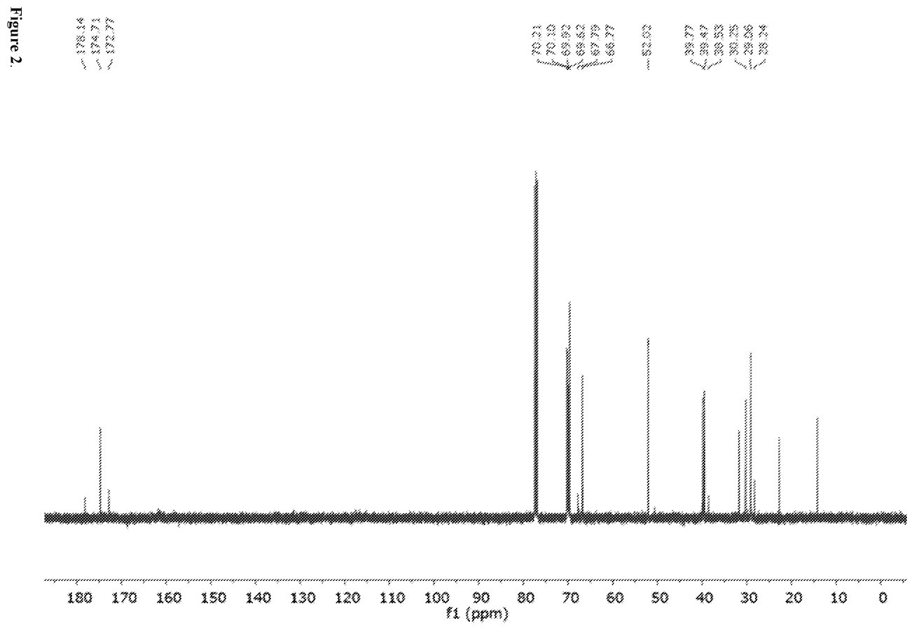

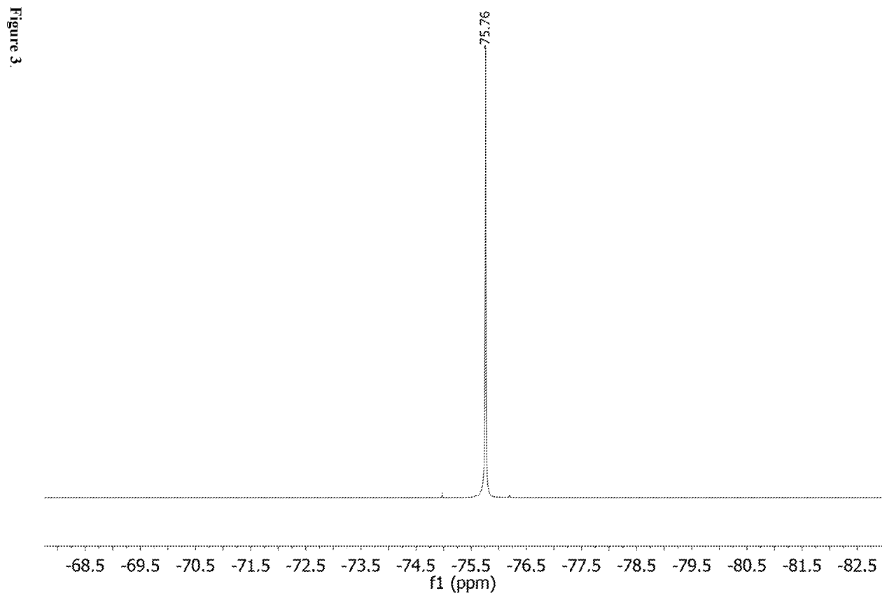

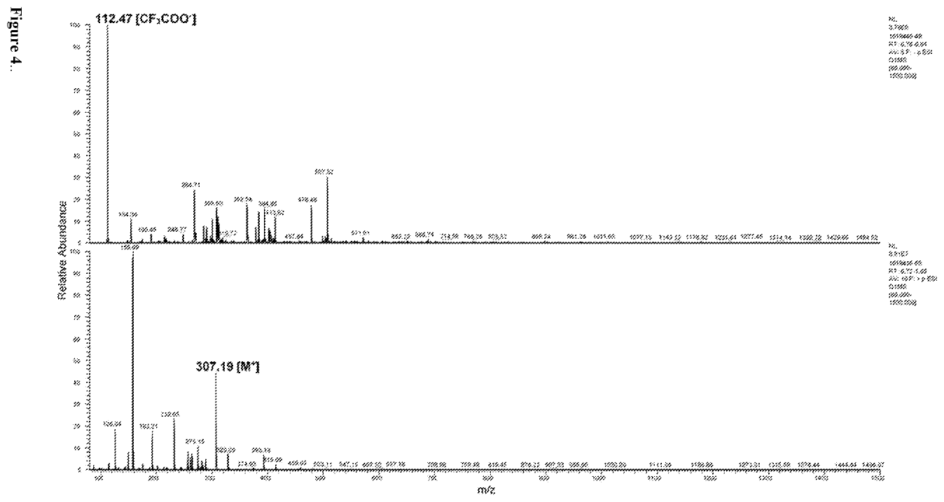

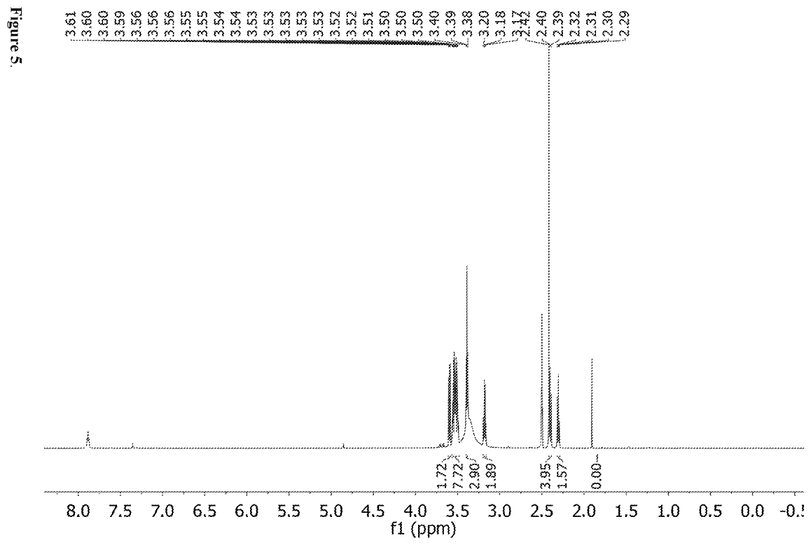

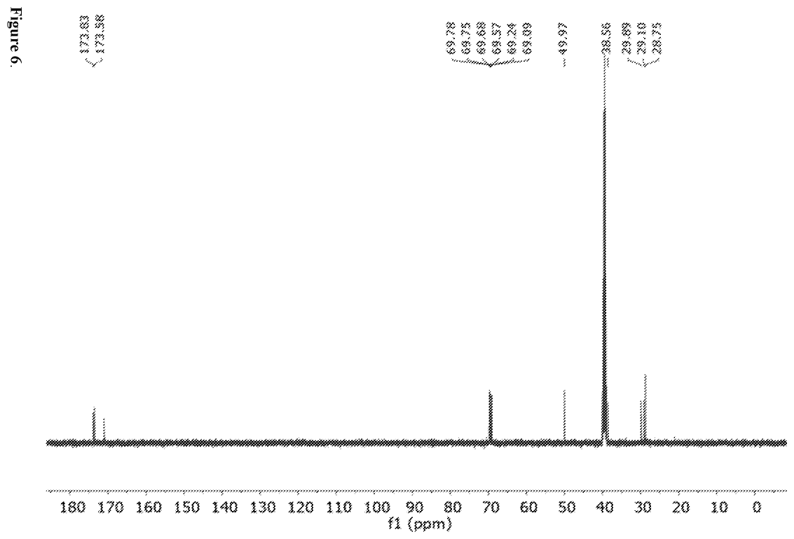

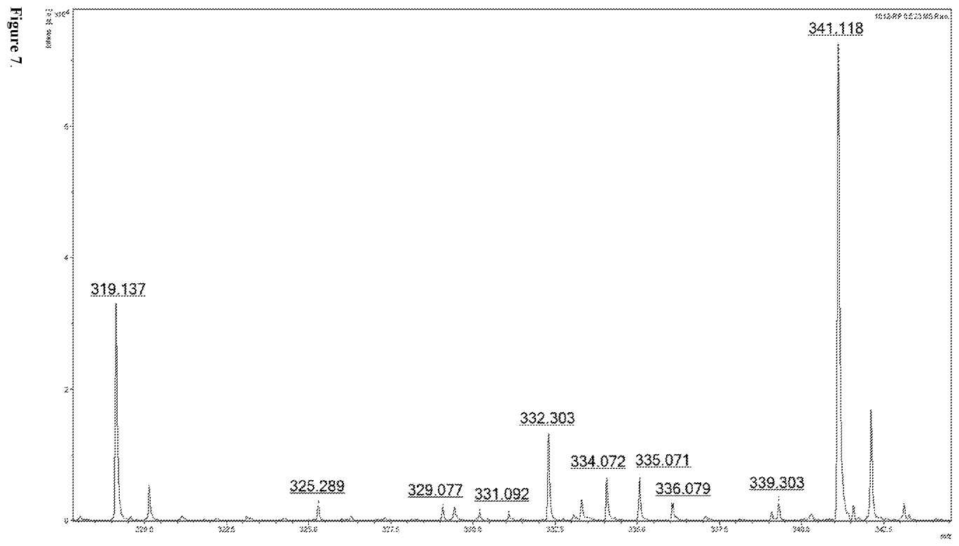

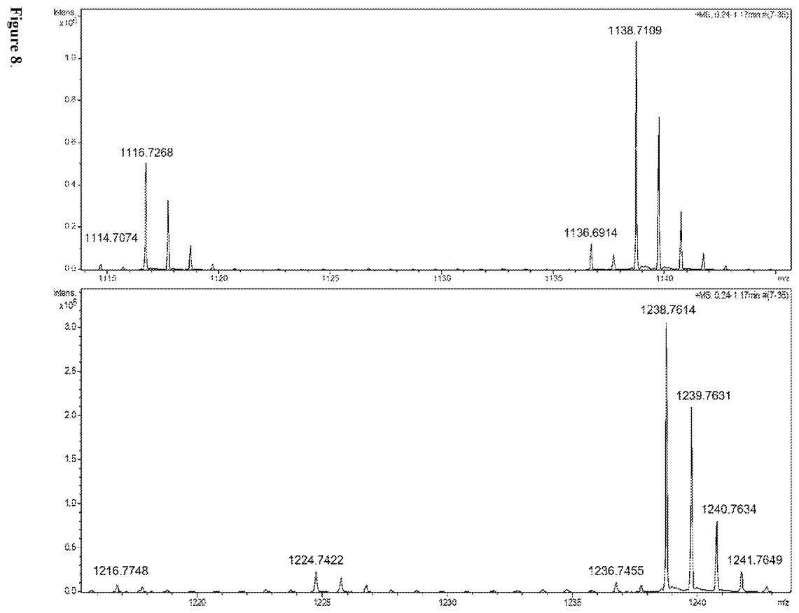

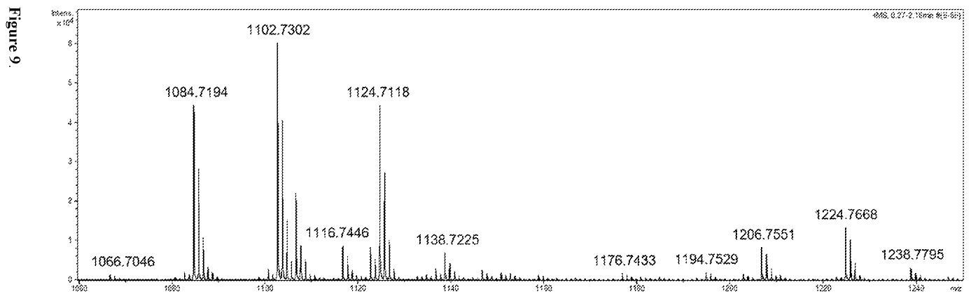

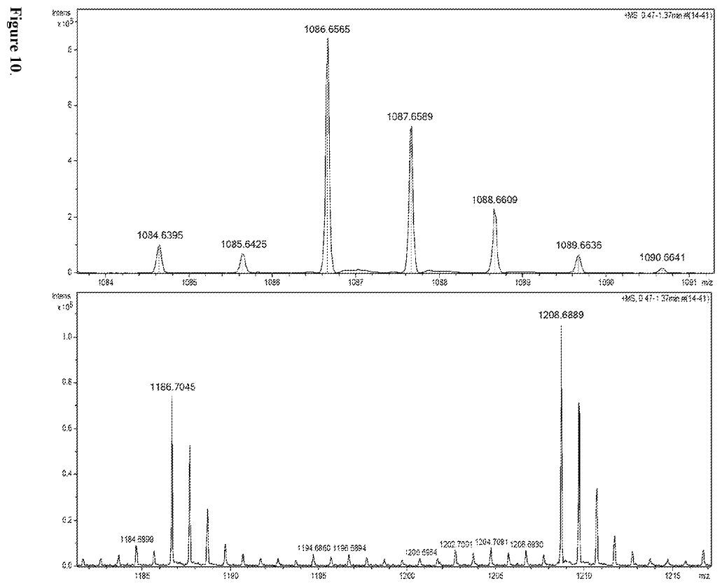

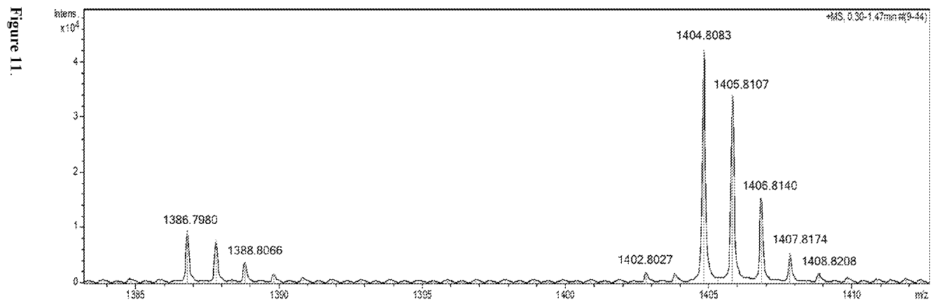

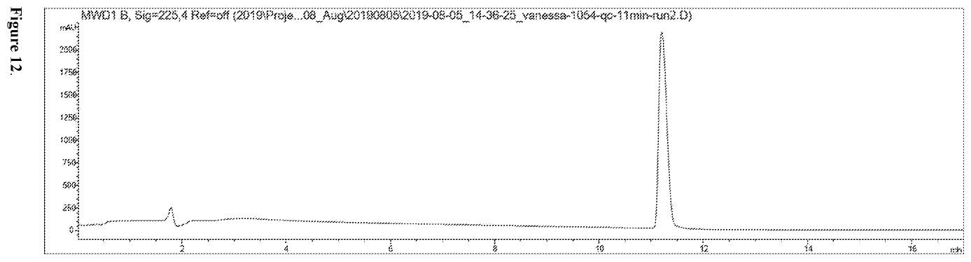

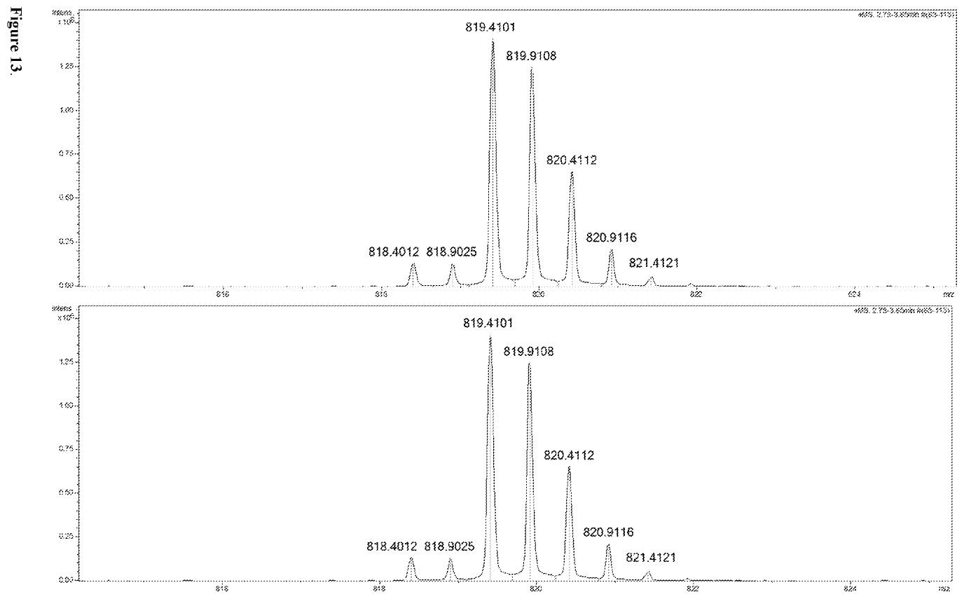

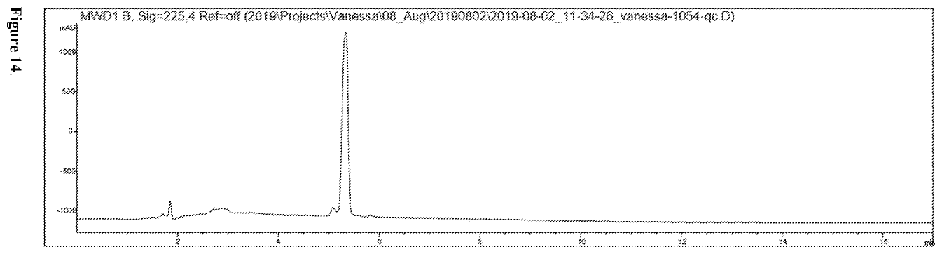

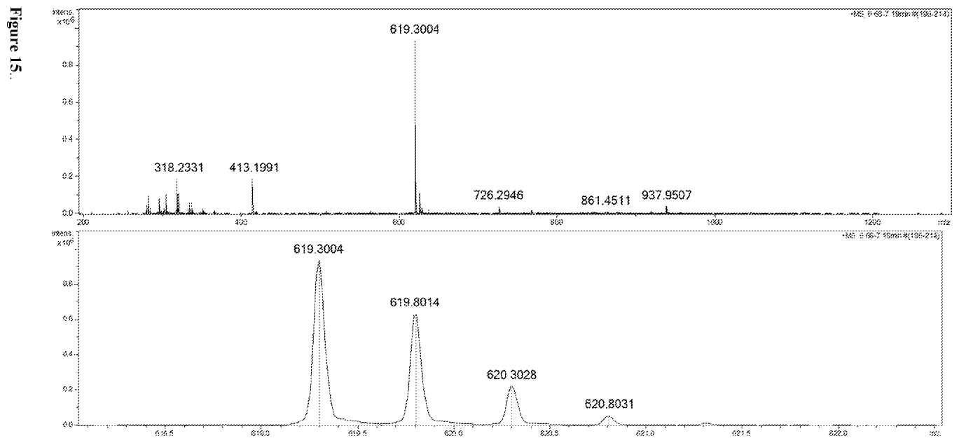

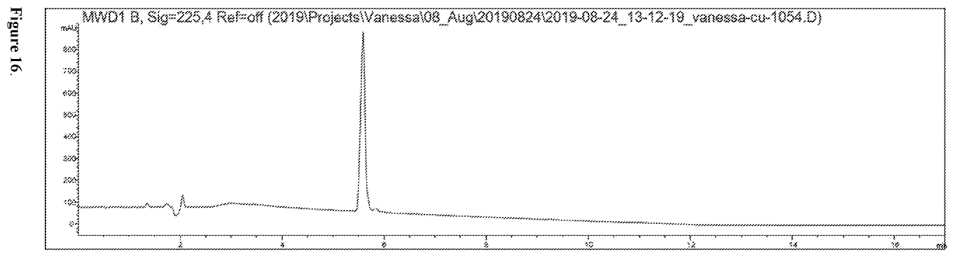

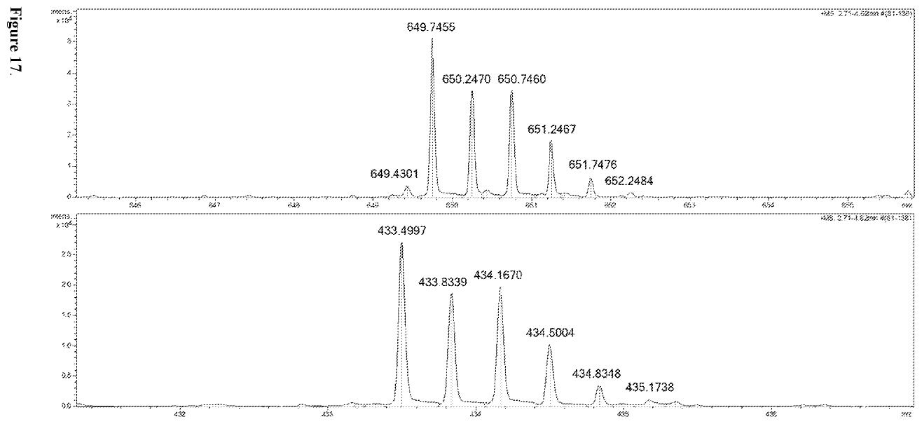

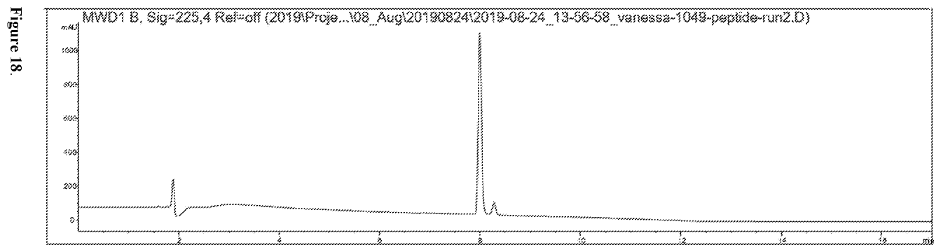

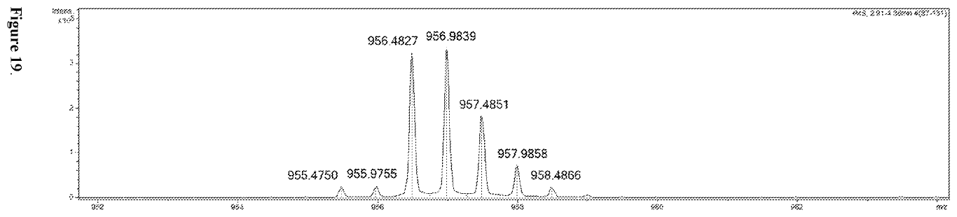

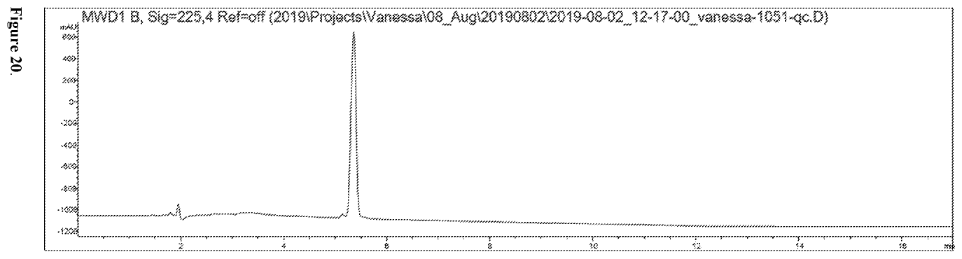

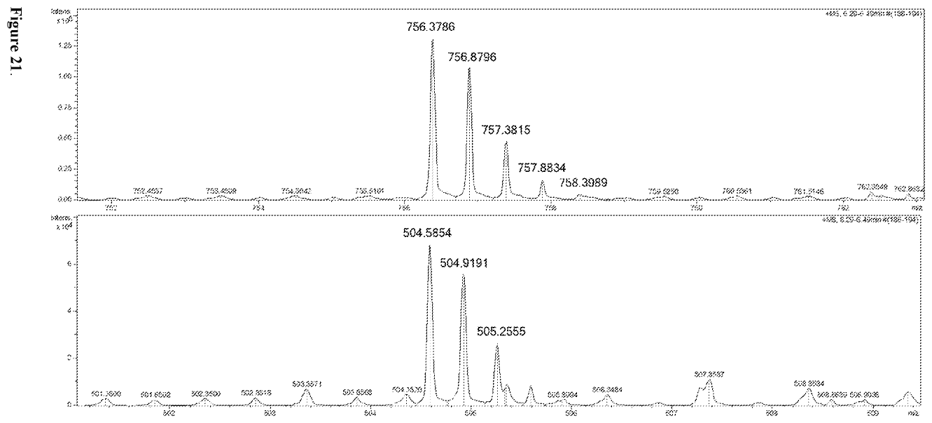

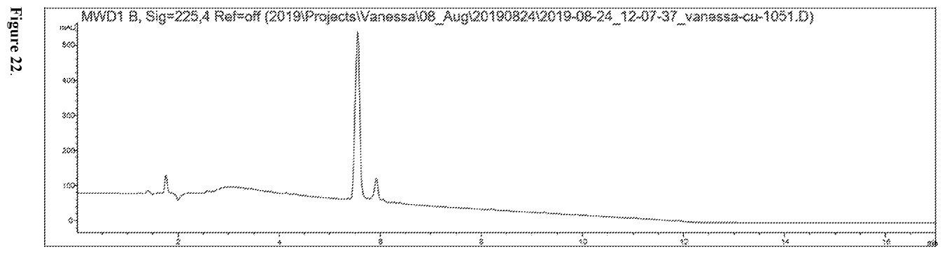

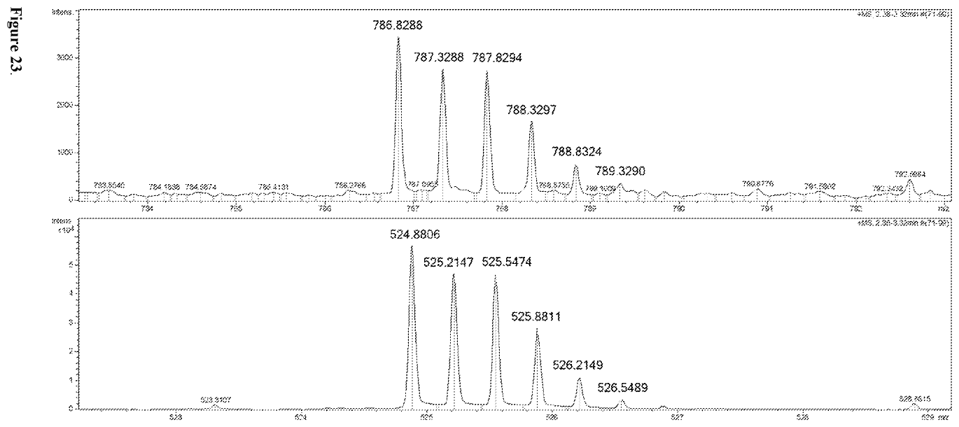

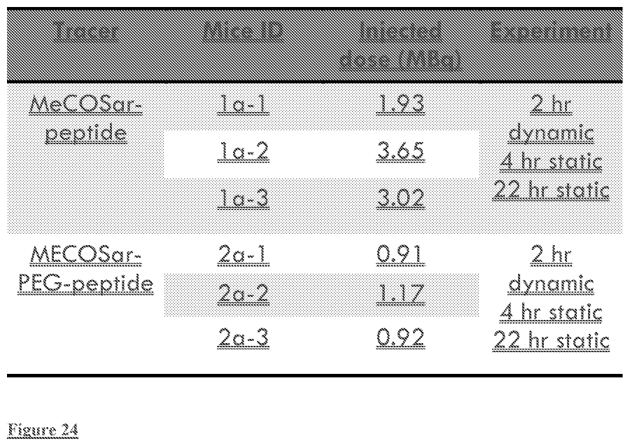

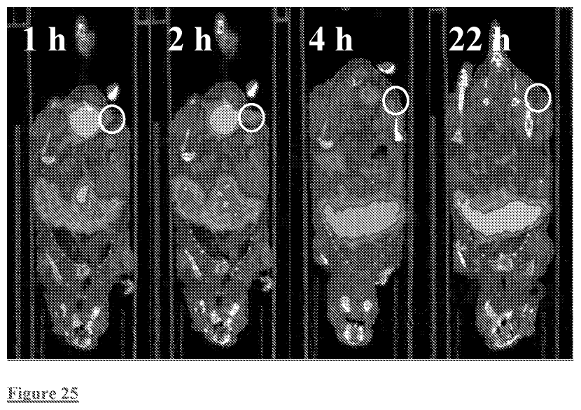

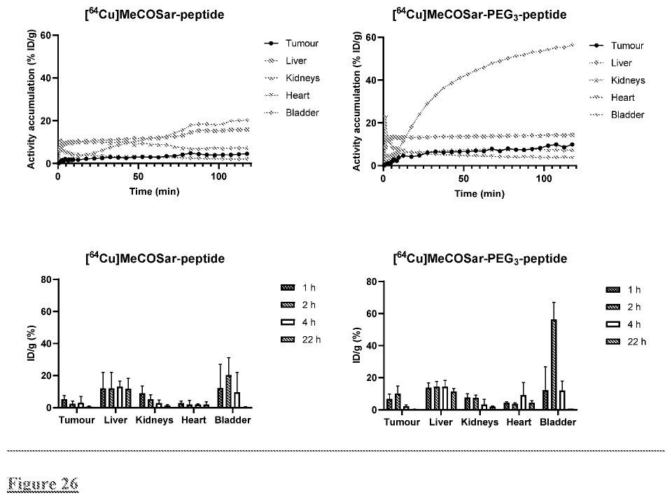

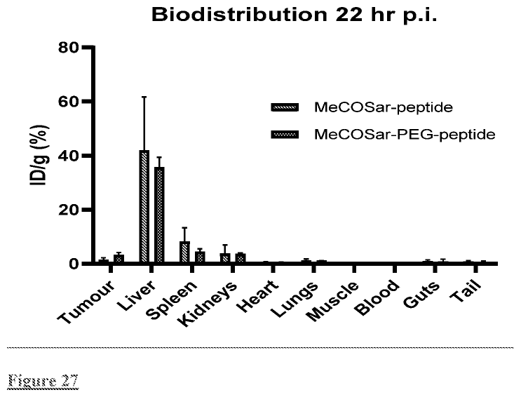

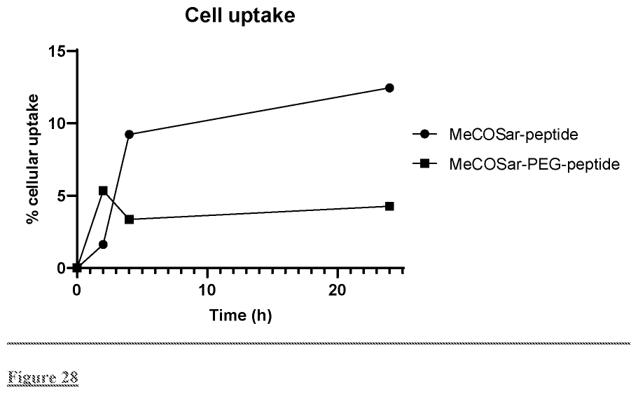

### DETAILED DESCRIPTION OF THE INVENTION

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

As used herein, the term “alkyl” refers to a monovalent alkyl groups that may be straight chained or branched, and preferably have from 1 to 12 carbon atoms, or more preferably 1 to 6 carbon atoms. Examples of such groups include methyl, ethyl, n-isopropyl, iso-propyl, n-butyl, iso-butyl, n-hexyl, and the like.

As used herein, the term “alkenyl” as a group or part of a group denotes an aliphatic hydrocarbon group containing at least one carbon-carbon double bond and which may be straight or branched preferably having 2-12 carbon atoms, more preferably 2-10 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl.

As used herein, the term “alkynyl” as a group or part of a group means an aliphatic hydrocarbon group containing a carbon-carbon triple bond and which may be straight or branched preferably having from 2-12 carbon atoms, more preferably 2-10 carbon atoms, more preferably 2-6 carbon atoms in the normal chain. Exemplary structures include, but are not limited to, ethynyl and propynyl.

As used herein, the term “cycloalkyl” refers to cyclic alkyl groups having a single cyclic ring or multiple condensed rings, preferably incorporating 3 to 8 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, and the like.

As used herein, the term “alkoxy” refers to the group “—O-alkyl”, wherein the alkyl groups is described above.

As used herein, the term “alkylene” refers to divalent alkyl groups preferably having from 1 to 12 carbon atoms and more preferably 1 to 6 carbon atoms, and even more preferably 1 to 3 carbon atoms. Examples of such alkylene groups include methylene (—CH₂—), ethylene (—CH₂CH₂—), and the propylene isomers (e.g., —CH₂CH₂CH₂— and —CH(CH₃)CH₂—), and the like.

As used herein, the term “acyl” refers to groups such as H—C(O)—, alkyl-C(O)—, cycloalkyl-C(O)— and aryl-C(O)— where alkyl, cycloalkyl and aryl are as described herein.

As used herein, the term “amino” refers to an —NH₂ group. The amino group may be optionally substituted, where the one or more hydrogen atoms of the group may be substituted with a group, such as an alkyl, cycloalkyl, aryl or heteroaryl group. The term “optionally substituted amino” refers to an amino group that bears further substitution.

As used herein, the term “amide” refers to a functional group consisting of a carbonyl group attached to a nitrogen atom. The term “optionally substituted amide” refers to an amide functional group that bears further substitution.

As used herein, the term “halogen” refers to the groups fluoro, chloro, bromo and iodo.

As used herein, the term “aryl” refers to a monovalent unsaturated aromatic carbocyclic group having a single ring (e.g. phenyl) or multiple condensed rings (e.g. naphthyl, anthracenyl), preferably having from 6 to 14 carbon atoms. Examples of aryl groups include phenyl, naphthyl, anthracenyl and the like.

As used herein, the term “optionally substituted” in relation to a particular group is taken to mean that the group may or may not be further substituted with one or more groups selected from hydroxyl, acyl, alkyl, alkoxy, alkenyl, alkenyloxy, alkynyl, alkynyloxy, amino, aminoacyl, alkylaryl, aryl, aryloxy, carboxyl, acylamino, cyano, halogen, nitro, sulphate, phosphate, phosphine, heteroaryl, heterocyclyl, oxyacyl, oxyacylamino, aminoacyloxy, trihalomethyl, and the like.

Examples of particularly suitable optional substituents include F, Cl, Br, I, CH₃, CH₂CH₃, OH, OCH₃, CF₃, OCF₃, NO₂, NH₂, COCH₃ and CN.

The compounds of Formula (I) to (IV) comprise a macrocyclic nitrogen-containing ligand also known as a “sarcophagine”. The sarcophagine of Formula (I) has the formula 5-(8-methyl-3,6,10,13,16,19-hexaaza-bicyclo[6.6.6]icosan-1-ylamino)-5-oxopentanoic acid and is also known as MeCOSar. The ligand contains six nitrogen atoms can coordinate a metal ion.

In certain embodiments, the compound of Formula (I), (II), (III) or (IV) is coordinated with a metal ion. In an embodiment, the metal ion is an ion of Cu, Tc, Gd, Ga, In, Co, Re, Fe, Au, Mg, Ca, Ag, Rh, Pt, Bi, Cr, W, Ni, V, Ir, Zn, Cd, Mn, Ru, Pd, Hg, Ti, Lu or Y.

In an embodiment, the compound of Formula (I) to (IV) is coordinated with a radionuclide. In some embodiments, the radionuclide is a metal ion of a metal selected from the group consisting of Cu, Tc, Ga, Co, In, Fe and Ti. The present inventors have found that the compounds herein disclosed are particularly useful in binding Cu ions. In an embodiment, the compound of Formula (I) is coordinated with a Cu ion. In another embodiment, the compound of Formula (I) is coordinated with a Cu ion that is a radionuclide. In some embodiments, the compound of Formula (I) is coordinated with a radionuclide selected from the group consisting of ⁶⁰Cu, ⁶²Cu, ⁶⁴Cu and ⁶⁷Cu. In one embodiment, the compound of Formula (I) to (IV) is coordinated with ⁶⁰Cu. In another embodiment, the compound of Formula (I) to (IV) is coordinated with ⁶²Cu. In another embodiment, the compound of Formula (I) to (IV) is coordinated with ⁶⁴Cu. In another embodiment, the compound of Formula (I) to (IV) is coordinated with ⁶⁷Cu. Where the compound of Formula (I) to (IV) is coordinated with a radionuclide and binds to a target receptor, the radiolabelled compound is in close proximity to the site at which the receptor is located. The radionuclide can undergo decay and the products of radioactive decay may then come into contact to the site at which the radiolabelled compound is bound.

The compounds of Formula (I) to (IV) comprise a cyclic polypeptide group attached to the sarcophagine via a linker group. Each of the components in the compound are linked together, however the manner and order in which they are linked together should ensure that the intended activity provided by each component is preserved in the compound. In other words, the compound comprising each of these compounds must be linked together in such a way so as to ensure that the activity of the radionuclide coordinated to the sarcophagine is maintained and the cyclic polypeptide binds to the intended receptor sufficiently.

The cyclic polypeptide group is a pentapeptide residue having the following structure:

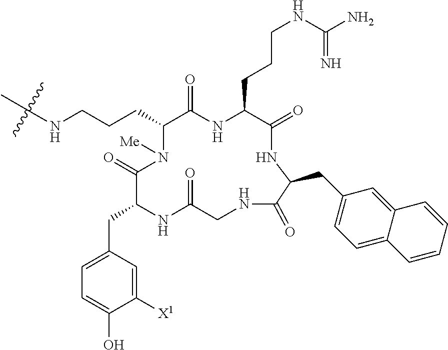

wherein X¹ is H or I. The cyclic pentapeptide has the sequence cyclo(D-Tyr-N-Me-D-Orn-L-Arg-L-2-Nal-Gly) and is attached to the linker group via the sidechain of the ornithine (Orn) residue. The pentapeptide has a variable X¹, which may be H or I. Peptides and residues of this nature may be prepared according to known methodologies. For instance, the peptides and their residues may be prepared by solid phase or solution phase procedures known in the art. In relation to the present invention, the peptide residue depicted is a group that is able to bind to the CXCR4 receptor, which is overexpressed on the surface of some cancer cells.

The linker group in the compounds of Formula (I) to (IV) acts as a spacer between the polypeptide and the sarcophagine and maintains a distance between the site at which the polypeptide binds and the radionuclide coordinated with the compound of Formula (I) to (IV). The length of the linker group is affected by the structure of the linker group. The distance between the site at which the polypeptide binds and the radionuclide should be optimised so as to ensure that the radioactivity provided by the radionuclide is localised to the binding site. The appropriate distance may depend on the nature of the receptor to which the polypeptide is to bind, the nature of the polypeptide itself and also the radionuclide that is complexed within the compound itself. The linking group should be such that it does not participate in any side reactions with the radionuclide, other functional groups present in the compound or in vivo.

Without wishing to be bound by theory, the present inventors have found that the overall length of the compounds of the present invention is affected by the nature of the linker group and the size and shape of the polypeptide and sarcophagine components of the compounds. The linker maintains a degree of separation between the polypeptide and sarcophagine, such that the distance between the polypeptide and sarcophagine is suitable for delivery of the radionuclide to the site at which the polypeptide binds. It is desirable that the degree of separation provided by the linker group is such that the activity of the polypeptide, i.e. binding to the target site, and the sarcophagine bound with the radionuclide do not interfere with each other. The present inventors believe that the combination of the polypeptide, the linker and the sarcophagine containing a radionuclide allows for the delivery of the radionuclide and the associated radioactivity to the surface of a cancer cell that expresses the receptor to which the polypeptide may bind. Upon binding of the compound to the surface of an appropriate cell, the radionuclide coordinated within the sarcophagine is maintained at a distance away from the cell, with the distance dictated by the nature of the linker group of the compound. The distance between the radionuclide and the cell of the surface is such that the radioactivity delivered by the radionuclide is sufficient to reach the cell surface.

In the compounds of the present invention, the terminal positions of the sarcophagine contain a propylamide group. The propylamide group may also be considered a linker and contributes to the separation of the sarcophagine containing the radionuclide and the polypeptide when bound to the surface of the cell. Since the polypeptide in the compounds are bound via the sidechain of the ornithine residue, i.e. the propylamine group, this sidechain also contributes to the overall distance between sarcophagine and the polypeptide. This may in turn mean that the linker group in the compounds of the present invention may be selected based on the desired length of the compound after taking into account the length provided by the propylamide group adjacent to the sarcophagine and the propylamine group of the polypeptide. The present inventors have found that connecting the linker and the polypeptide via a different amino acid, i.e. via the arginine (Arg) or tyrosine (Tyr) residues, would result in a compound of a different size and length. Where the polypeptide and linker are connected differently, in addition to the compounds having a different size, shape and length, the binding properties of the polypeptide to the target receptor may be different as the nature of the sidechains influences the ability of the polypeptide to bind. In turn, whether a particular functional group on the polypeptide is occupied by binding or is free and unbound influences the stability of the overall compound. For instance, if the polypeptide was not bound via the propylamine sidechain as depicted above, the amine group on the sidechain would lead to the compound having a reactive primary amine group exposed. Since primary amine groups are known for their considerable reactivity, the overall compound may in fact be unstable. Therefore it is important that the polypeptide is bound to the sarcophagine and/or linker in the correct manner.

The present invention provides compounds of Formula (I) to (IV) containing a pentixafor or pentixather polypeptide and a sarcophagine bound by a linker.

The compound of Formula (I) contains a single polypeptide and a single sarcophagine unit.

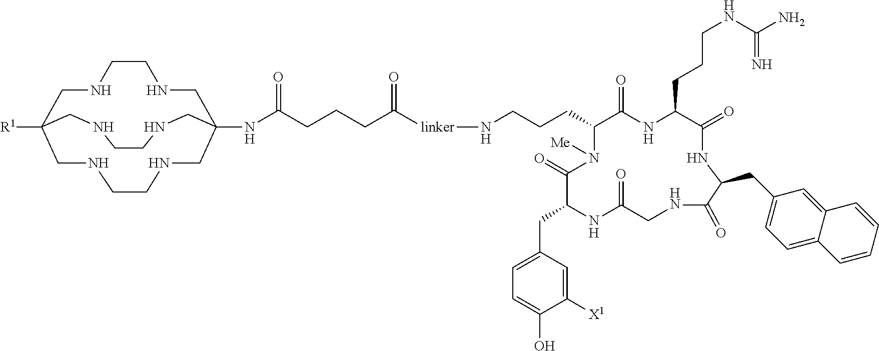

In certain embodiments of the compound of Formula (I), the linker is absent or selected from

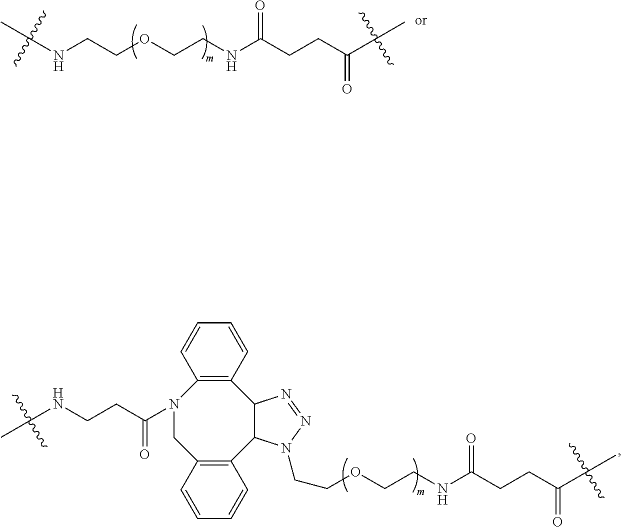

wherein m is an integer from 1 to 10.

In an embodiment, the linker is absent and the compound of Formula (I) has the following structure:

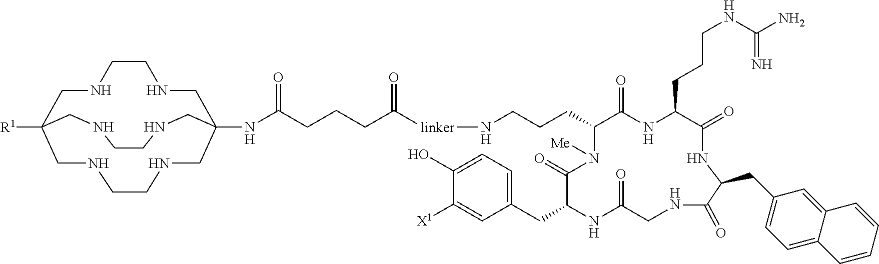

wherein R¹ and X¹ have the definitions above.

The group represented by variable R¹ is at the terminal position of the sarcophagine cage. Where the group R¹ is a reactive functional group, for example, an amino group, the compound may be further functionalised through reactions with appropriate coupling partners. In an embodiment, R¹ is optionally substituted amino. In an embodiment, R¹ is optionally substituted C₁-C₁₂ alkyl. In another embodiment, R¹ is optionally substituted C₁ alkyl. In a preferred embodiment, R¹ is unsubstituted C₁ alkyl. In a preferred embodiment, R¹ is methyl.

One skilled in the art would understand that the linkers for the compounds of Formula (I) may be joined to the other components of the compound at either end. In an embodiment, the end of the linker indicated with a * is attached to the propylamide group of the sarcophagine:

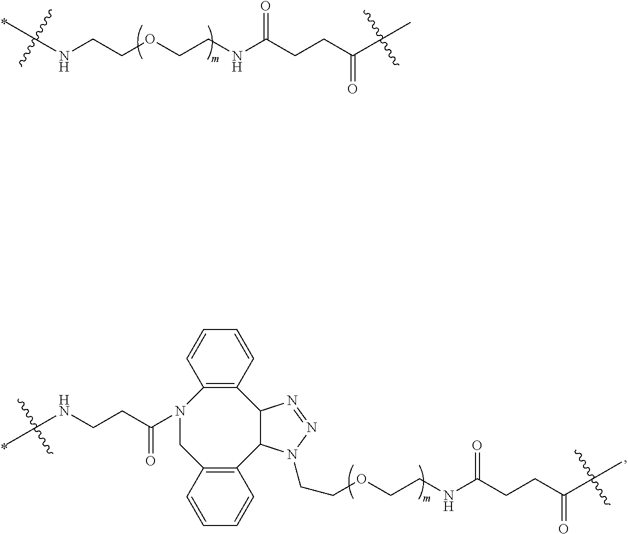

In an embodiment, the compound of Formula (I) has the following structure:

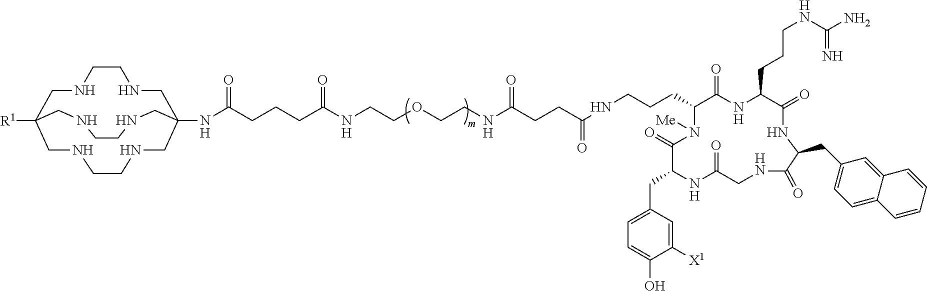

wherein R¹, X¹ and m have the definitions above. This embodiment of Formula (I) contains one or more polyethylene glycol (PEG) units in the linker. The number of PEG units in the compound affects the overall length of the compound of Formula (I). In some embodiments, m is an integer from 1 to 10. In some embodiments, m is an integer selected from the group consisting of 1, 2, 3, 4, 5, 6, 7, 8, 9 and 10. In an embodiment, X¹ is H. In another embodiment, X¹ is I.

In another embodiment, the compound of Formula (I) has the following structure:

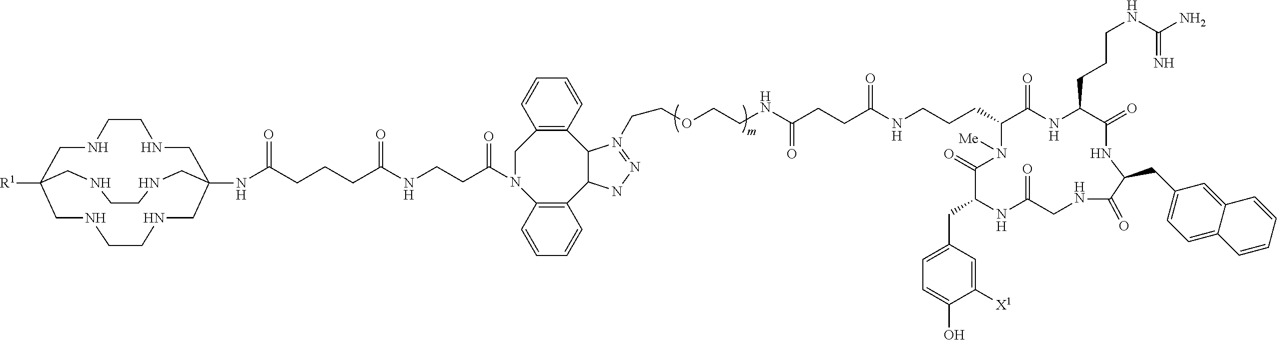

wherein R¹, X¹ and m have the definitions above. This embodiment of Formula (I) contains a cyclooctene-triazole unit and one or more PEG units in the linker. In some embodiments, m is an integer from 1 to 10. In some embodiments, m is an integer selected from the group consisting of 1, 2, 3, 4, 5, 6, 7, 8, 9 and 10. In an embodiment, X¹ is H. In another embodiment, X¹ is I.

The present invention also provides compounds of Formula (II), which contain a single sarcophagine unit two linker and polypeptide units. The linker and polypeptide units are bound to the terminal positions of the sarcophagine and the compounds have the following structure:

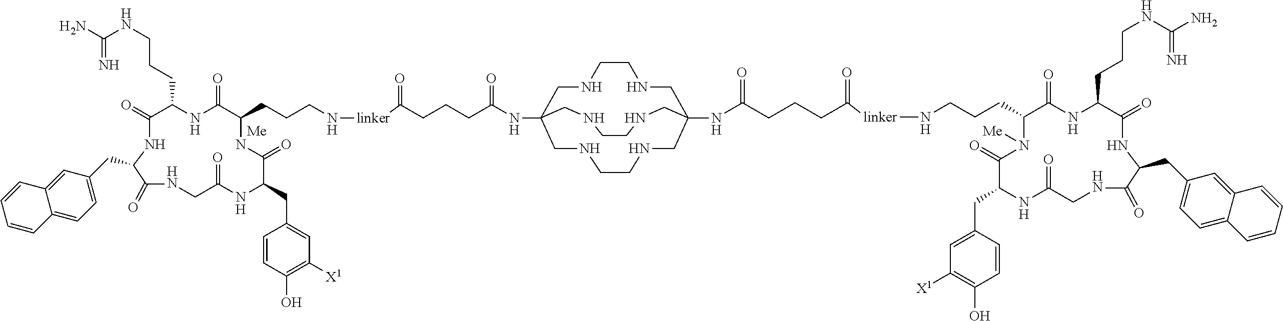

wherein X¹ has the definition above.

Since the compounds contain two polypeptide units that are capable of binding to the CXCR4 receptor on a cell surface, the compounds of Formula (II) may show increased binding affinity. In some cases, one polypeptide unit in the compound of Formula (II) may bind to the cell surface. In other cases both polypeptide units in the compound of Formula (II) may be bound.

As for the compounds of Formula (I), the linkers for compounds of Formula (II) may be joined to the other components of the compound at either end. In an embodiment, the end of the linker indicated with a * is attached to the propylamide group of the sarcophagine:

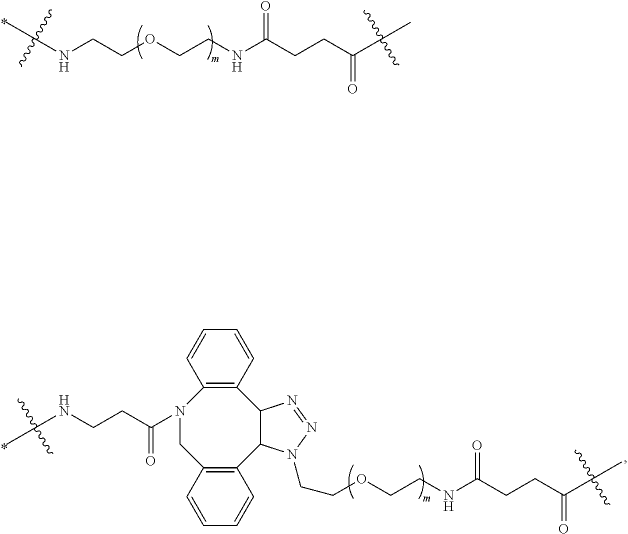

In an embodiment, the compound of Formula (II) has the following structure:

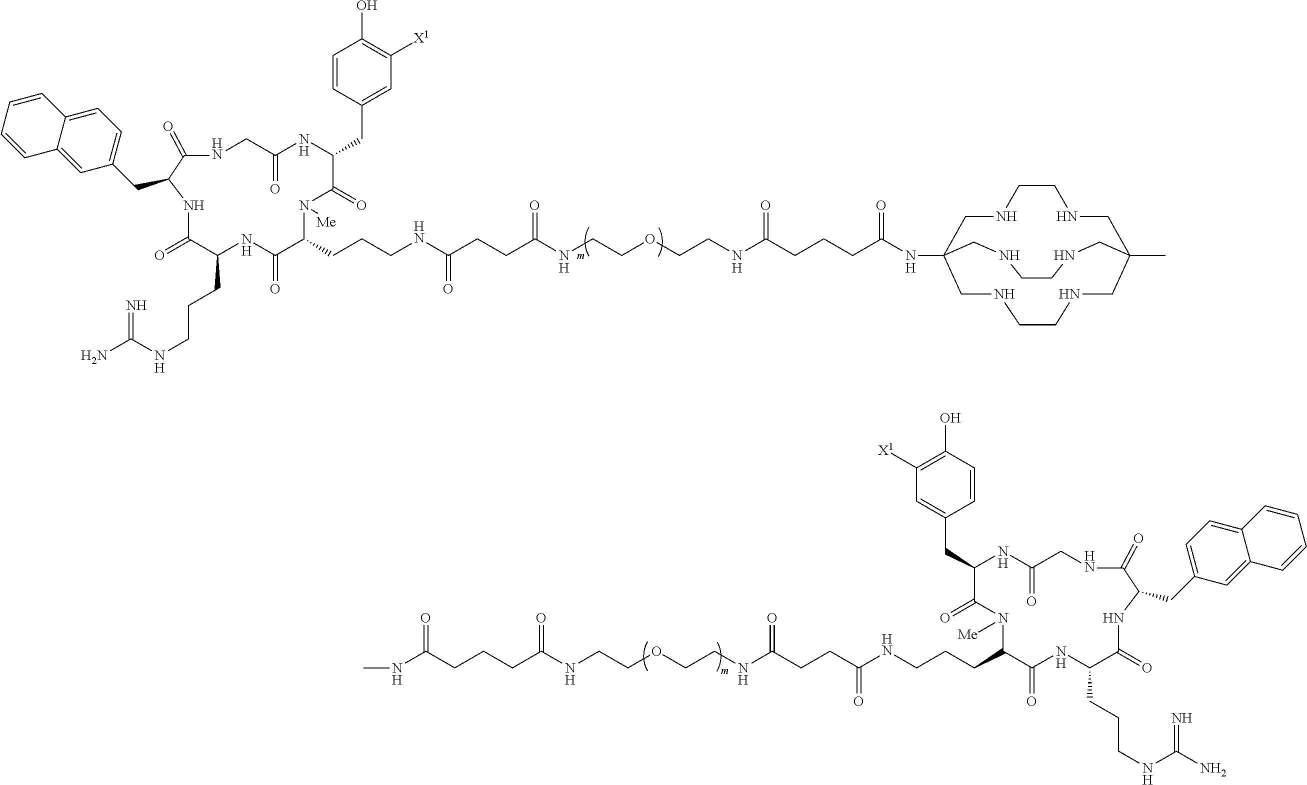

wherein X¹ and m have the definitions above. This embodiment of Formula (II) contains one or more PEG units in each linker. The number of PEG units in the compound affects the overall length of the compound of Formula (II). In some embodiments, m is independently an integer from 1 to 10. In some embodiments, m is independently an integer selected from the group consisting of 1, 2, 3, 4, 5, 6, 7, 8, 9 and 10. In some embodiments, each occurrence of m is the same. In some embodiments, each occurrence of m is different. In an embodiment, X¹ is H. In another embodiment, X¹ is I.

In another embodiment, the compound of Formula (II) has the following structure:

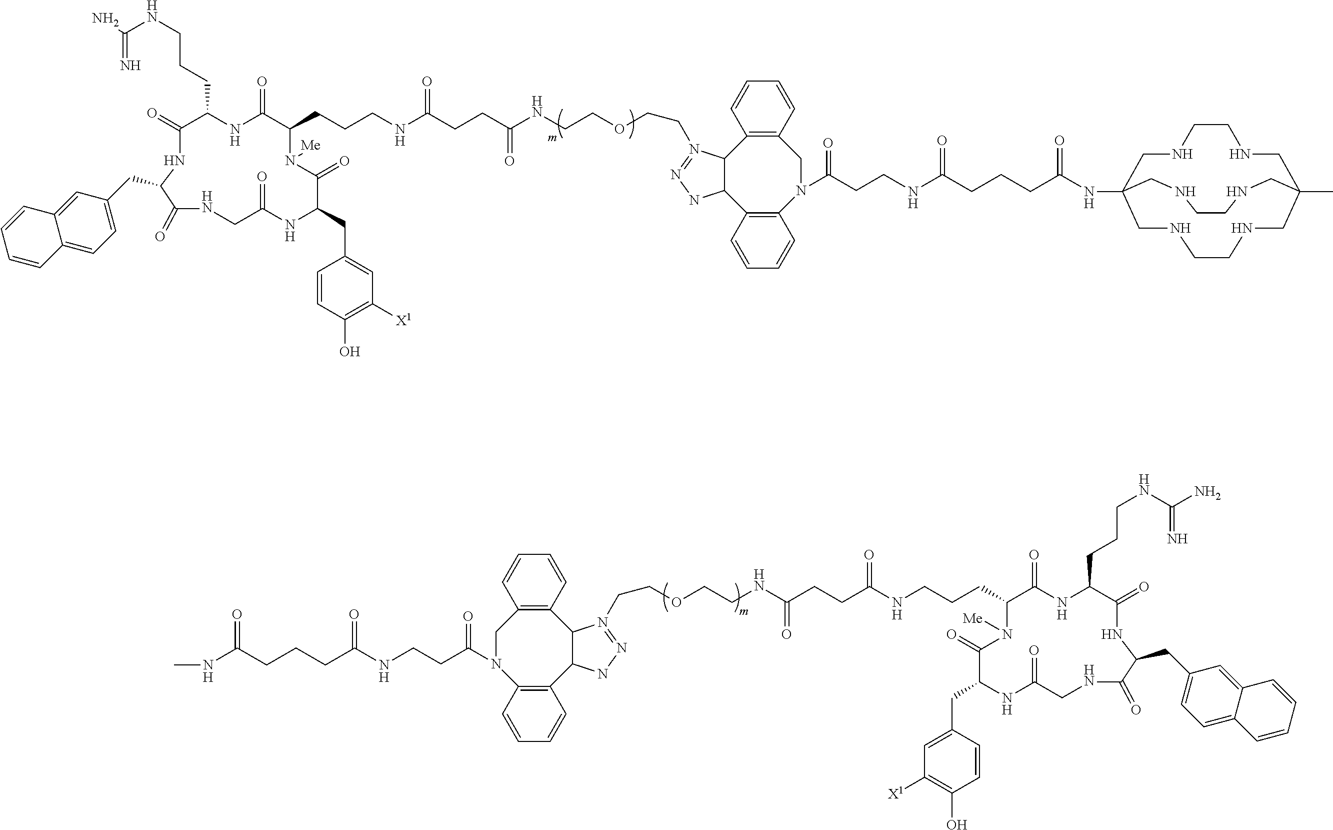

wherein X¹ and m have the definitions above. This embodiment of Formula (II) contains two cyclooctene-triazole units and two PEG units in the linker. In some embodiments, m is independently an integer from 1 to 10. In some embodiments, m is independently an integer selected from the group consisting of 1, 2, 3, 4, 5, 6, 7, 8, 9 and 10. In some embodiments, each occurrence of m is the same. In some embodiments, each occurrence of m is different. In an embodiment, X¹ is H. In another embodiment, X¹ is I.

The present invention also provides compounds of Formula (III), which contain a single linker unit bound to the sarcophagine, however the linker unit also binds two polypeptide units. The linker is bound to a terminal position of the sarcophagine and subsequently binds two different polypeptide units. This means that compounds of Formula (III) may have a better overall binding affinity than compounds containing a single polypeptide unit.

The compound of Formula (III) has the following structure:

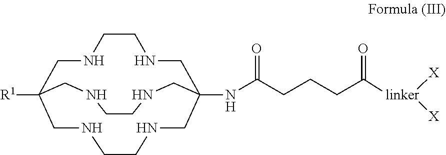

wherein:

R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;

X is

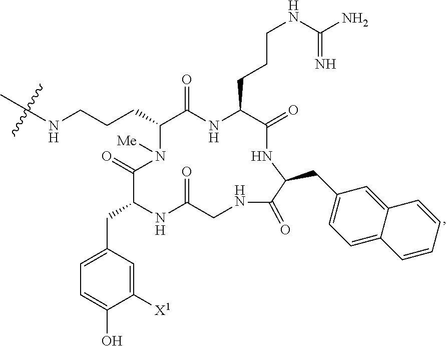

wherein X¹ is H or iodo; and

the linker is selected from:

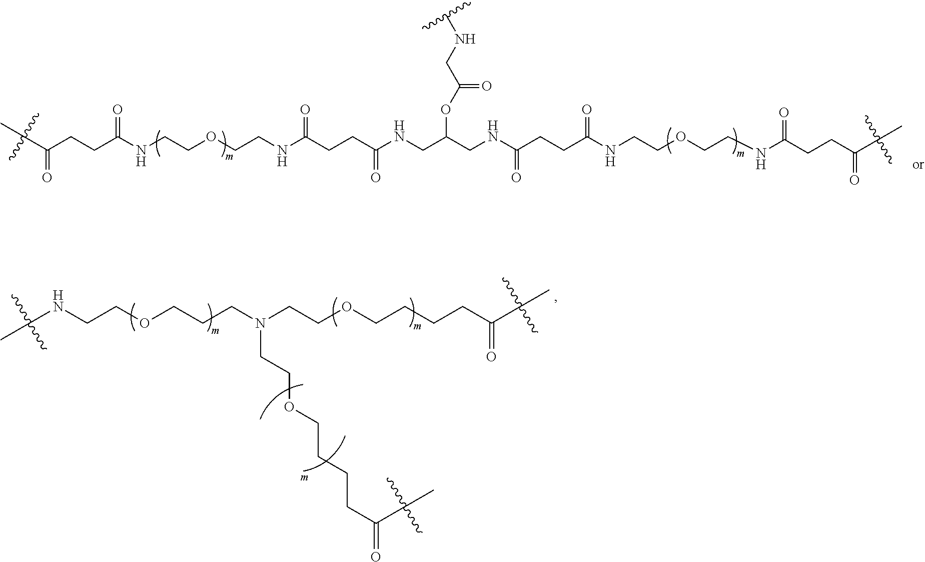

wherein m is independently an integer from 1 to 10.

As for the compounds of Formula (I), the group represented by variable R¹ in the compound of Formula (III) is at the terminal position of the sarcophagine cage. Where the group R¹ is a reactive functional group, for example, an amino group, the compound may be further functionalised through reactions with appropriate coupling partners. In an embodiment, R¹ is optionally substituted amino. In an embodiment, R¹ is optionally substituted C₁-C₁₂ alkyl. In another embodiment, R¹ is optionally substituted C₁ alkyl. In a preferred embodiment, R¹ is unsubstituted C₁ alkyl. In a preferred embodiment, R¹ is methyl.

As for the compounds of Formula (I) and (II), the linkers of Formula (III) may be joined to the other components of the compound at either end. In an embodiment, the end of the linker indicated with a * is attached to the propylamide group of the sarcophagine:

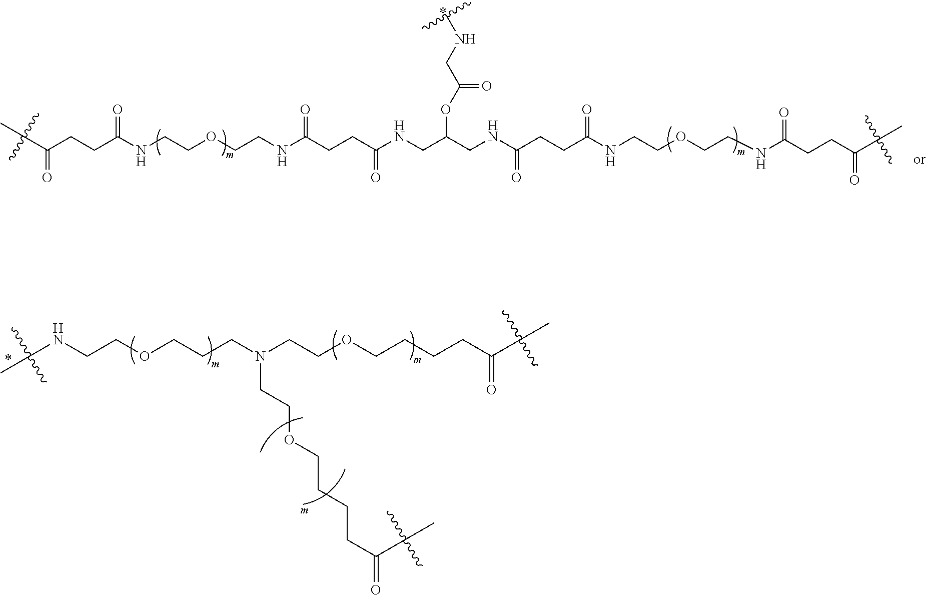

In an embodiment, the compound of Formula (III) has the following structure:

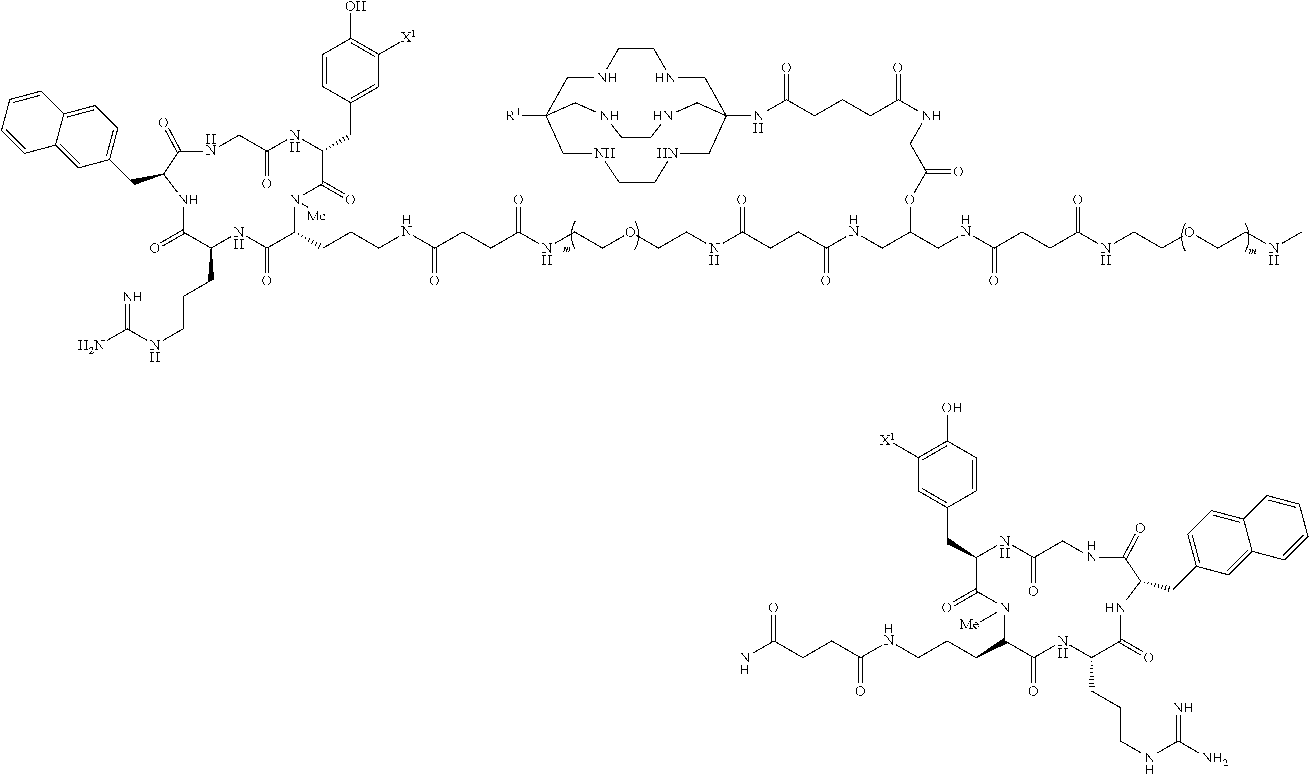

wherein R¹, X and m have the definitions above. In this embodiment of Formula (III), the compound contains a single linker containing one or more PEG groups interspersed with ethylamide-type groups. In addition to the ethylamide groups, the linker contains a propylamide group bound directly to the sarcophagine. The linker group in this embodiment, contains a tertiary carbon centre, which results in the compound of Formula (III) having a roughly two-armed structure. This may mean that each of the polypeptide units occupy a different area of space and may allow for greater flexibility in binding. In some embodiments, m is independently an integer from 1 to 10. In some embodiments, m is independently an integer selected from the group consisting of 1, 2, 3, 4, 5, 6, 7, 8, 9 and 10. In some embodiments, each occurrence of m is the same. In some embodiments, each occurrence of m is different.

In an embodiment, the compound of Formula (III) has the following structure:

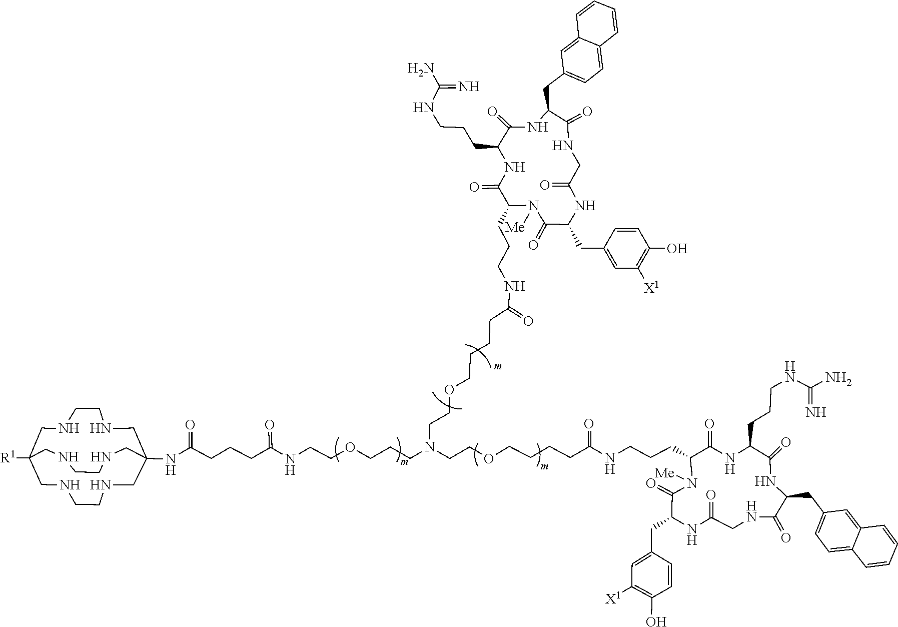

wherein R¹, X¹ and m have the definitions above. In this embodiment of Formula (III), the compound also contains a single linker containing one or more PEG groups. The PEG groups are located around a tertiary nitrogen centre and also provides the compound of Formula (III) with a roughly two-armed structure. The linker also contains a propylamide group bound directly to the sarcophagine. In some embodiments, m is independently an integer from 1 to 10. In some embodiments, m is independently an integer selected from the group consisting of 1, 2, 3, 4, 5, 6, 7, 8, 9 and 10. In some embodiments, each occurrence of m is the same. In some embodiments, each occurrence of m is different. In an embodiment, X¹ is H. In another embodiment, X¹ is I.

The present invention also provides compounds of Formula (IV), which contain a single linker unit bound to the sarcophagine and where the linker then binds three units of the polypeptide. Since compounds of Formula (IV) have three units of the polypeptide that are capable of binding to a receptor, compounds of Formula (IV) may show a greater overall binding affinity when compared to other compounds containing fewer units of the polypeptide.

The compounds of Formula (IV) have the following structure:

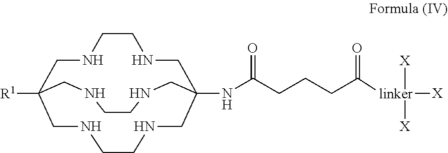

wherein:

R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;

X is

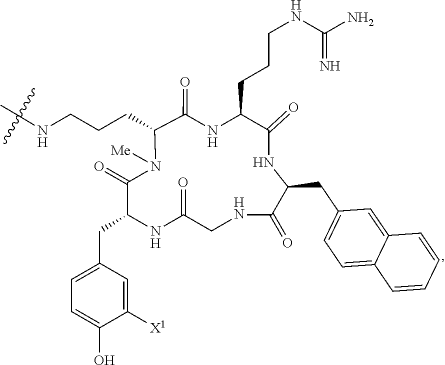

wherein X¹ is H or iodo; and

the linker is selected from:

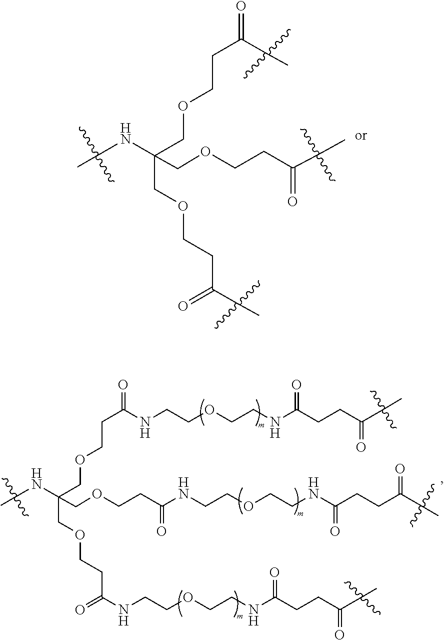

wherein m is an integer from 1 to 10.

As for the compounds of Formula (I) to (III), the linkers of Formula (IV) may be joined to the other components of the compound at several points. In an embodiment, the end of the linker indicated with a * is attached to the propylamide group of the sarcophagine:

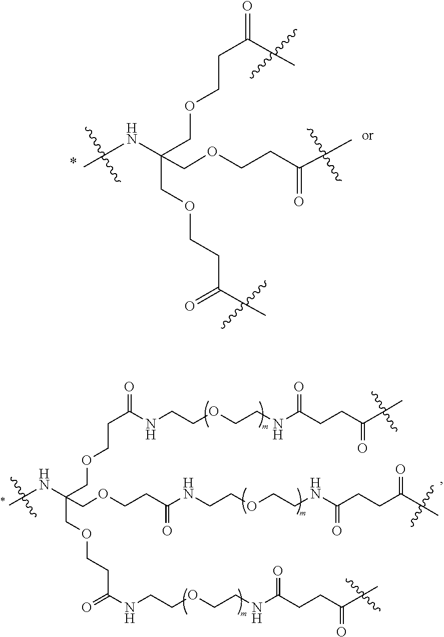

In an embodiment, the compound of Formula (IV) has the following structure:

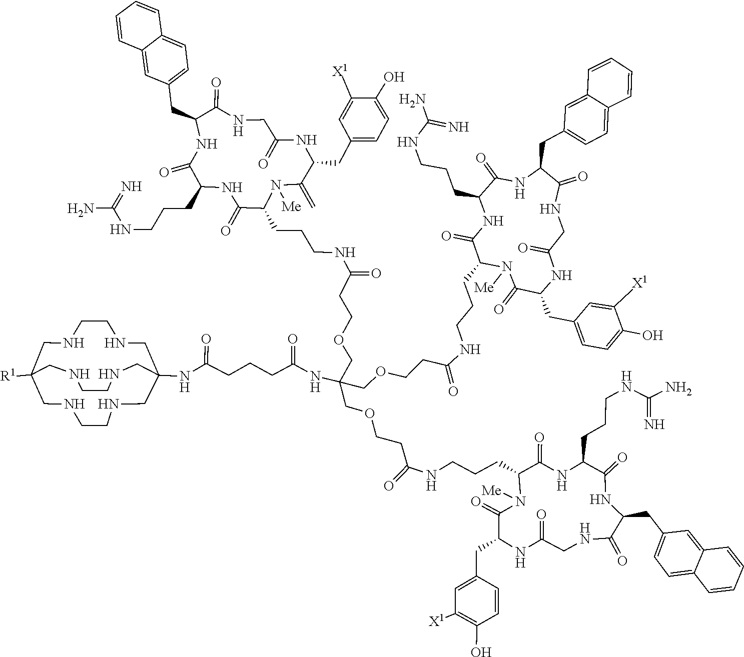

wherein R¹ and X¹ have the definitions above. In this embodiment of Formula (IV), the linker contains a carbon atom to which three alkylene ether groups are bound. Each of the three polypeptide units is bound an arm of the linker, thus creating a roughly three-armed structure.

Each polypeptide unit is capable of binding to a receptor. In some embodiments, only one polypeptide unit of a compound of Formula (IV) is bound to a receptor. In other embodiments, more than one polypeptide unit of a compound of Formula (IV) is bound to a receptor. Where more than one polypeptide unit binds to a receptor, the overall binding of the compound to the binding site may be stronger than compounds having a single polypeptide unit or compounds where only single polypeptide unit is capable of binding. In an embodiment, X¹ is H. In another embodiment, X¹ is I.

In another embodiment, the compound of Formula (IV) has the following structure:

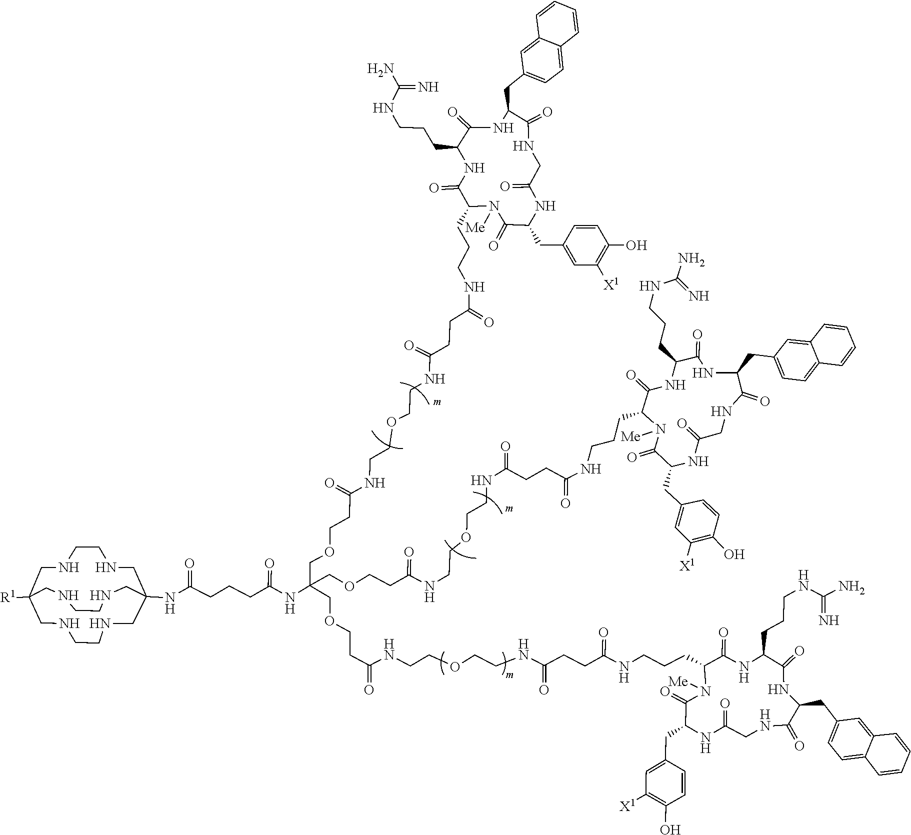

wherein R¹, X¹ and m have the definitions above. In these embodiments, the linker contains a carbon atom to which three linear chains containing amide groups, alkylene groups and PEG groups are bound. These compounds of the present invention have a roughly three armed structure and contain three polypeptide units bound to each linear chain of the linker. In some embodiments, m is independently an integer from 1 to 10. In some embodiments, m is independently an integer selected from the group consisting of 1, 2, 3, 4, 5, 6, 7, 8, 9 and 10. In some embodiments, each occurrence of m is the same. In some embodiments, each occurrence of m is different. In an embodiment, X¹ is H. In another embodiment, X¹ is I.

As used herein, the term “pharmaceutically acceptable salts” refers to salts that retain the desired biological activity of the parent compound, and include pharmaceutically acceptable acid addition salts and base addition salts. Suitable pharmaceutically acceptable acid addition salts of compounds of Formula (I) may be prepared from an inorganic acid or an organic acid. Examples of an inorganic acid include hydrochloric acid, sulphuric acid and phosphoric acid. Examples of organic acids include aliphatic, cycloaliphatic, aromatic, heterocyclic carboxylic and sulfonic organic acids, such as, formic, acetic, proprionic, succinic, glycolic, gluronic, lactic, malic, tartaric, citric, fumaric, maleic, alkylsulfonic and arylsulfonic acids. Where the compound of Formula (I) is a solid, the compounds and salts thereof may exist in one or more different crystalline or polymorphic forms, all of which are intended to be within the scope of Formula (I).

In an embodiment, the present invention provides compositions comprising a compound as described above together with one or more pharmaceutically acceptable excipients.

Pharmaceutical compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

The present invention provides a method for the radioimaging of a subject, the method comprising administering a therapeutically effective amount of a compound as described herein or a composition as described herein.

The compounds of the present invention may be radiolabelled with a radionuclide or a radioisotope that undergoes spontaneous decay. Where these byproducts of decay are detected by means such as positron emission tomography (PET) or single-photon emission computed tomography (SPECT), images showing the localisation of the radiolabelled compounds may be obtained. The images may then be used in the diagnosis of various conditions, where the location of the radiolabelled compound is reflected in the image. The radiolabelled compounds according to the present invention localise in particular areas because the cyclic peptide fragment of the compound shows an affinity for a receptor that may be expressed or overexpressed in a particular area. Where the expression or overexpression of a particular receptor is characteristic for a particular condition, the identification of these localised areas in the generated image may contribute to the diagnosis of the condition.

In an embodiment, the method for radioimaging of a subject comprises the administration of an effective amount of a compound of Formula (I) to (IV) or a salt thereof, wherein the compound contains a radionuclide. In an embodiment, the compound of Formula (I) to (IV) or a salt thereof is coordinated with a radionuclide that is a Cu ion. In some embodiments, the compound of Formula (I) to (IV) or a salt thereof is coordinated with a radionuclide selected from the group consisting of ⁶⁰Cu, ⁶²Cu, ⁶⁴Cu and ⁶⁷Cu. In one embodiment, the compound of Formula (I) to (IV) or a salt thereof is coordinated with ⁶⁰Cu. In another embodiment, the compound of Formula (I) to (IV) or a salt thereof is coordinated with ⁶²Cu. In another embodiment, the compound of Formula (I) to (IV) or a salt thereof is coordinated with ⁶⁴Cu. In another embodiment, the compound of Formula (I) to (IV) or a salt thereof is coordinated with ⁶⁷Cu.

The present invention also provides a method of treating or preventing a condition in a subject, the method comprising administering a therapeutically effective amount of a compound as described herein or a composition as described herein.

The radiolabelled compounds of the present invention may also be used for treating or preventing a condition in a subject. Where the radiolabelled compounds of the present invention contain a radionuclide capable of undergoing radioactive decay, the localisation of the radiolabelled compound exposes the immediate area to the decay products. Where the radiolabelled compound is bound to a cancer or a tumour site, owing to the expression or overexpression of a receptor for which the peptide has an affinity, the compound may be useful in the treatment of the tumour or cancer by radiotherapy.

In an embodiment, the method for treating or preventing a condition in a subject comprises the administration of a therapeutically effective amount of a compound of Formula (I) to (IV) or a salt thereof, wherein the compound contains a radionuclide. In an embodiment, the compound of Formula (I) to (IV) or a salt thereof is coordinated with a radionuclide that is a Cu ion. In some embodiments, the compound of Formula (I) to (IV) or a salt thereof is coordinated with a radionuclide selected from the group consisting of ⁶⁰Cu, ⁶²Cu, ⁶⁴Cu and ⁶⁷Cu. In one embodiment, the compound of Formula (I) to (IV) or a salt thereof is coordinated with ⁶⁰Cu. In another embodiment, the compound of Formula (I) to (IV) or a salt thereof is coordinated with ⁶²Cu. In another embodiment, the compound of Formula (I) to (IV) or a salt thereof is coordinated with ⁶⁴Cu. In another embodiment, the compound of Formula (I) to (IV) or a salt thereof is coordinated with ⁶⁷Cu.

A therapeutically effective amount can be readily determined by an attending clinician by the use of conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular radio labelled compound administered, the mode of administration, the bioavailability of the preparation administered, the dose regime selected, the use of other medications and other relevant circumstances.

In addition, the treatment regime will typically involve a number of cycles of radiation treatment with the cycles being continued until such time as the condition has been ameliorated. Once again the optimal number of cycles and the spacing between each treatment cycle will depend upon a number of factors such as the severity of the condition being treated, the health (or lack thereof) of the subject being treated and their reaction to radiotherapy. In general the optimal dosage amount and the optimal treatment regime can be readily determined by a skilled addressee in the art using well known techniques.

The compounds of the invention may be administered in any form or mode which makes the compound available for the desired application (imaging or radio therapy). One skilled in the art of preparing formulations of this type can readily select the proper form and mode of administration depending upon the particular characteristics of the compound selected, the condition to be treated, the stage of the condition to be treated and other relevant circumstances. We refer the reader to Remington's Pharmaceutical Sciences, 19th edition, Mack Publishing Co. (1995) for further information.

The compounds of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds are, however, typically used in the form of pharmaceutical compositions which are formulated depending on the desired mode of administration. The compositions are prepared in manners well known in the art.

The invention in other embodiments provides a pharmaceutical pack or kit comprising one or more containers filled with one or more of the ingredients of the pharmaceutical compositions of the invention. In such a pack or kit can be found at least one container having a unit dosage of the agent(s). Conveniently, in the kits, single dosages can be provided in sterile vials so that the clinician can employ the vials directly, where the vials will have the desired amount and concentration of compound and radio nucleotide which may be admixed prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

The compounds of the invention may be used or administered in combination with one or more additional drugs that are anti-cancer drugs and/or procedures (e.g. surgery, radiotherapy) for the treatment of the disorder/diseases mentioned. The components can be administered in the same formulation or in separate formulations. If administered in separate formulations the compounds of the invention may be administered sequentially or simultaneously with the other drugs.

In addition to being able to be administered in combination with one or more additional drugs that include anti-cancer drugs, the compounds of the invention may be used in a combination therapy. When this is done the compounds are typically administered in combination with each other. Thus one or more of the compounds of the invention may be administered either simultaneously (as a combined preparation) or sequentially in order to achieve a desired effect. This is especially desirable where the therapeutic profile of each compound is different such that the combined effect of the two drugs provides an improved therapeutic result.

As discussed above, the compounds of the present invention may be useful for treating and/or detecting conditions such as a cancer. The compounds of the present invention may be particularly useful for treating and/or detecting tumours such as breast cancer, colon cancer, lung cancer, ovarian cancer, prostate cancer, head and/or neck cancer, or renal, gastric, pancreatic cancer and brain cancer as well as hematologic malignancies such as lymphoma and leukaemia. In addition, the compounds of the present invention may be useful for treating and/or detecting a cancer that is refractory to the treatment and/or detecting with other anti-cancer drugs; and for treating and/or detecting hyperproliferative conditions such as leukaemias, psoriasis and restenosis. In other embodiments, compounds of this invention can be used to treat and/or detect pre-cancer conditions or hyperplasia including familial adenomatous polyposis, colonic adenomatous polyps, myeloid dysplasia, endometrial dysplasia, endometrial hyperplasia with atypia, cervical dysplasia, vaginal intraepithelial neoplasia, benign prostatic hyperplasia, papillomas of the larynx, actinic and solar keratosis, seborrheic keratosis and keratoacanthoma. In an embodiment, the cancer is breast cancer. In an embodiment, the cancer may be associated with a tumour.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

### EXAMPLES

Synthesis of the Compounds of the Invention

The agents of the various embodiments may be prepared using the reaction routes and synthesis schemes as described below, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T.W. Greene's Protective Groups in Organic Synthesis, 3rd Edition, John Wiley & Sons, 1991. Alternatively, other reactions disclosed herein or known in the art will be recognized as having applicability for preparing other compounds of the various embodiments.

General Experimental Details

All reagents and solvents were obtained from commercial sources and used as received. 5-(8-methyl-3,6,10,13,16,19-hexaaza-bicyclo[6.6.6]icosan-1-ylamino)-5-oxopentanoic acid (MeCOSar) was provided by Clarity Pharmaceuticals (Sydney, Australia). The compound (t-Boc)₄₋₅MeCOSar was synthesized using a previously reported procedure. The peptide (cyclo(D-3-iodo-Tyr¹-[NMe]-D-Orn²-Arg³-2-Nal⁴-Gly⁵) was obtained from WuXi Apptec (Shanghai, China). Peptide conjugation and synthesis of non-radioactive reference compounds were carried out using previously reported procedures.

NMR spectra were recorded on a Bruker Avance 500 spectrometer. The chemical shifts were internally referenced to the residual solvent signals relative to tetramethylsilane (¹H, ¹³C) or externally to CF₃CO₂H (¹⁹F). High resolution ESI−MS mass spectra were acquired using a Bruker Micro TOF Q II spectrometer. MALDI-TOF spectra were acquired using a Bruker Daltonics Autoflex Speed device.

Purification of the peptides was performed by semi-preparative HPLC on a Agilent Eclipse XDB-C18 5u (9.4×250 mm) column applying a gradient of 5-95% (B) in 17 min at a flow rate of 4 mL/min ((A) 0.1% TFA in water; (B) acetonitrile). Ultraviolet detection was performed using a Agilent 35900E Series II detector at λ=225 nm. Quality control of the peptides was performed by analytical HPLC on a Agilent Eclipse Plus C18 (150×4.6 mm) column using the same gradient as previously described at a flow rate of 1 mL/min.

### Example 1—Synthesis of Heterobifunctional PEG Linkers

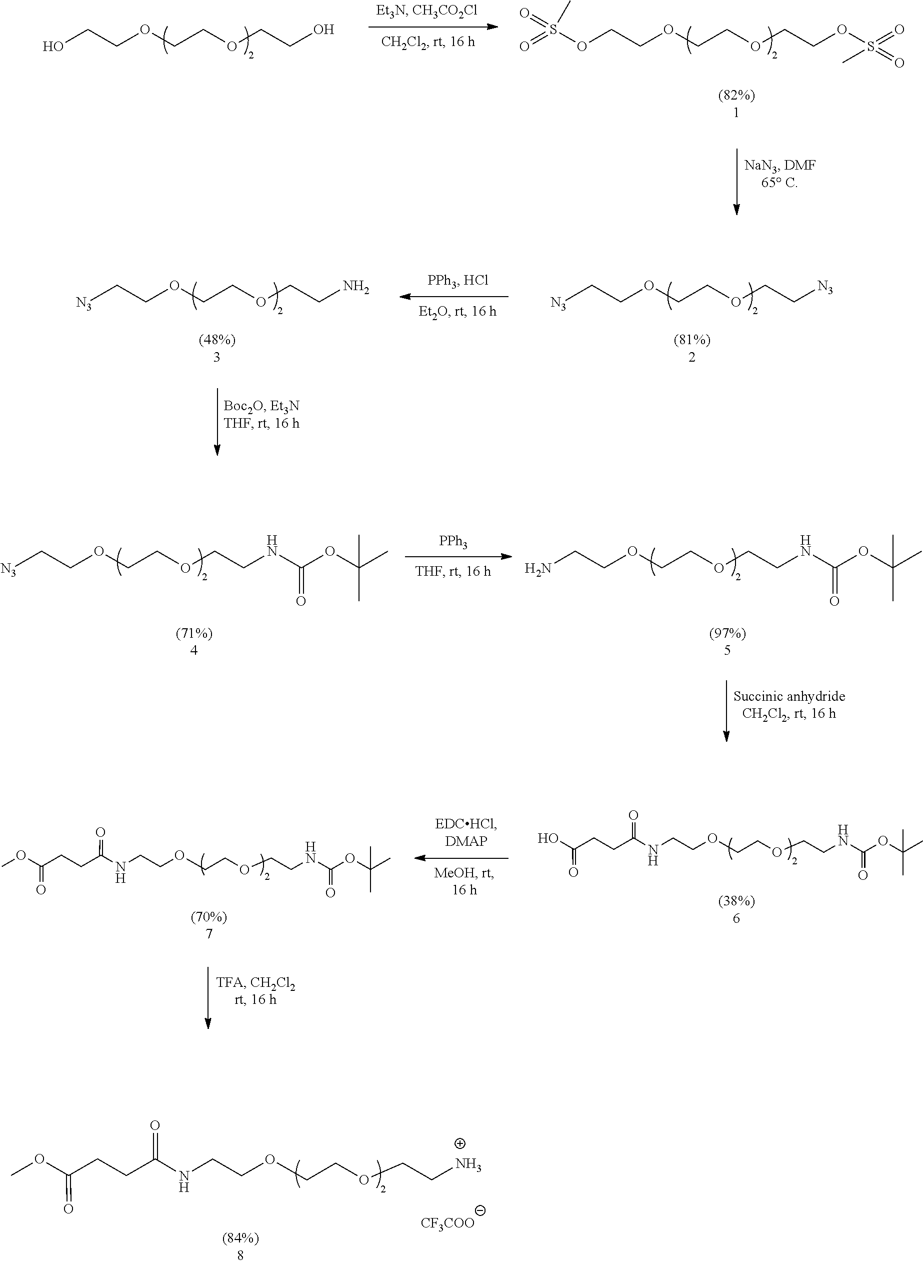

((Oxybis(ethane-2,1-diyl))bis(oxy))bis(ethane-2,1-diyl) dimethanesulfonate (1). Tetraethylene glycol (10 g, 0.0515 mol) was dissolved in dry DCM (50 mL) and combined with triethylamine (15 mL, 0.107 mol) under nitrogen at 0° C. Methanesulfonyl chloride (12.6 g, 0.109 mol) was dissolved in dry DCM (10 mL) and added dropwise to the reaction mixture over 30 min. The reaction was allowed to stir at room temperature for 24 h under a nitrogen flow. The resulting reaction mixture was then washed with a 3% HCl solution followed by brine. The organic layers were combined and dried over anhydrous sodium sulfate. The suspension was filtered, and solvents removed in vacuo affording the product as a yellow oil (14.7 g, 82% yield). ¹H NMR (CDCl₃, 500 MHz) δ 3.07 (s, 6H), 3.62-3.67 (m, 8H), 3.76 (t, 4H, J=5.0 Hz), 4.37 (t, 4H, J=5.0 Hz); ¹³C NMR (CDCl₃, 125 MHz) δ 38.3 (CH₃), 69.7, 69.9, 71.2, 71.3 (CH₂).

1-Azido-2-(2-(2-(2-azidoethoxy)ethoxy)ethoxy)ethane (2). Compound 1 (14.7 g, 0.0420 mol) was combined with sodium azide (5.46 g, 0.0420 mol) in dry DMF (50 mL) under nitrogen. The reaction was allowed to stir at 65° C. overnight. The reaction mixture was diluted with diethyl ether (50 mL) and washed with water (3×50 mL) and brine (2×50 mL). The organic layers were collected and dried over anhydrous sodium sulfate, and solvents removed in vacuo affording the product as a colourless oil (8.31 g, 66% yield). ¹H NMR (CDCl₃, 500 MHz) δ 3.31 (t, 4H, J=5.0 Hz), 3.59-3.61 (m, 12H); ¹³C NMR (CDCl₃, 125 MHz) δ 51.1 (CH₂), 70.4 (NCH₂), 71.1 (OCH₂).

1-Azido-2-(2-(2-(2-azidoethoxy)ethoxy)ethoxy)ethan-1-amine (3). Compound 2 (3.43 g, 0.0140 mol) was dissolved in 5% HCl (40 mL). Triphenylphosphine (3.67 g, 0.0140 mol) was dissolved in diethyl ether, and added dropwise to the reaction mixture using an automated syringe pump set (10 mL/h). The reaction mixture was allowed to stir at room temperature for 24 h under a nitrogen flow. The resulting mixture was washed with ethyl acetate (3×50 mL). The aqueous layers were combined and pH adjusted to 12 using NaOH (2M, 80 mL). The aqueous layer was extracted with DCM. The organic layers were combined and dried over anhydrous sodium sulphate. Solvents were removed in vacuo to afford the product as a slightly yellowish oil (1.45 g, 47% yield). ¹H NMR (CDCl₃, 500 MHz) δ 1.06 (t, 2H, J=5.0 Hz), 1.96 (br, 2H), 2.71 (t, 2H, J=5.0 Hz), 3.25 (t, 2H, J=5.0 Hz), 3.37 (t, 2H, J=5.0 Hz), 3.49-3.55 (m, 10H). ¹³C NMR (CDCl₃, 125 MHz) δ 18.7, 42.0, 51.0, 57.8, 70.4, 70.6, 70.95, 71.01, 73.6 (CH₂).

tert-Butyl (2-(2-(2-(2-azidoethoxy)ethoxy)ethoxy)ethyl)carbamate (4). Compound 3 (3.58 g, 0.0164 mol) was combined with di-tert-butyl dicarbonate (3.94 g, 0.0180 mol) in dry THF (20 mL) at 0° C. Triethylamine (3 mL, 0.0213 mol) was added to the reaction mixture and the latter was brought to room temperature and allowed to stir overnight under a nitrogen flow. The resulting mixture was diluted with DCM and washed with sodium bicarbonate and brine. The organic layers were combined and dried over anhydrous sodium sulfate. Solvents were removed in vacuo. The crude product was purified with column chromatography (silica, neat DCM ˜3% v/v methanol in DCM), affording the product as a colourless oil (3.71 g, 71% yield). ¹H NMR (CDCl₃, 500 MHz) δ 1.42 (s, 9H), 3.30 (t, 2H, J=5.0 Hz), 3.37 (t, 2H, J=5.0 Hz), 3.52 (t, 2H, J=5.0 Hz), 3.60-3.68 (m, 10H), 5.02 (br, 1H); ¹³C NMR (CDCl₃, 125 MHz) δ 29.1, 41.0, 51.3, 70.7, 70.9, 71.26, 71.28, 71.4, 79.8, 156.6.

tert-Butyl (2-(2-(2-(2-aminoethoxy)ethoxy)ethoxy)ethyl)carbamate (5). Compound 4 (3.71 g, 0.0117 mol) was combined with triphenylphosphine (3.07 g, 0.0117 mol) and dissolved in THE (20 mL) and water (15 mL). The reaction was allowed to stir at room temperature overnight. The solvents were removed in vacuo and the residue dissolved in toluene and water. The aqueous layer was washed with toluene (3×50 mL) and solvents removed in vacuo. The product was afforded as a colourless oil (3.31 g, 97% yield). ¹H NMR (DMSO-d₆, 500 MHz) δ 1.37 (s, 9H), 2.66 (t, 2H, J=5.0 Hz), 3.06 (q, 2H, J=5.0 Hz), 3.37 (t, 4H, J=5.0 Hz), 3.50-3.52 (m, 8H); ¹³C NMR (DMSO-d₆, 125 MHz) δ 28.2, 41.1, 69.2, 69.5, 69.6, 72.5, 77.6, 155.6.

2,2-Dimethyl-4,18-dioxo-3,8,11,14-tetraoxa-5,7-diazahenicosan-21-oic acid (6). Compound 5 (3.31 g, 0.0113 mol) was combined with succinic anhydride (2.26 g, 0.226 mol) in dry DCM and allowed to stir overnight at room temperature. The reaction mixture was washed with water (3×50 mL) and brine (2×30 mL). The organic layers were combined and dried over anhydrous sodium sulfate. The crude product was purified by column chromatography (silica, neat DCM ˜3% v/v methanol in DCM with 0.5% v/v glacial acetic acid) affording compound 6 as a colourless oil (1.69 g, 38% yield). ¹H NMR (CDCl₃, 500 MHz) δ 1.44 (s, 9H), 2.52 (t, 2H, J=5.0 Hz), 2.67 (t, 2H, J=5.0 Hz), 3.31 (bs, 2H), 3.43-3.46 (m, 2H), 3.55 (t, 4H, J=5.0 Hz), 3.54-3.65 (m, 8H), 5.15 (bs, 1H), 6.67 (bs, 1H); ¹³C NMR (CDCl₃, 125 MHz) δ 28.5 (CH₃), 30.4, 31.2, 39.6, 40.4, 41.7, 69.8, 70.2, 70.4, 70.5, 70.7, 79.5 (CH₂), 156.3, 172.6, 175.1 (C═O). ESI−MS: calculated for C₁₇H₃₂N₂O₈: 392.2; found 391.1 [M−H].

Methyl 2,2-dimethyl-4,18-dioxo-3,8,11,14-tetraoxa-5,17-diazahenicosan-21-oate (7). Compound 6 (0.086 g, 0.219 mmol) was stirred in methanol (2 mL) and N-(3-dimethylaminopropyl)-N′-ethylcarbodiimide hydrochloride (0.043 g, 0.224 mmol) and 4-dimethylaminopyridine (0.00133 g, 0.01089 mmol) subsequently added to the reaction mixture. The reaction mixture was allowed to stir overnight at room temperature. The solvent was removed in vacuo. The mixture was diluted with DCM and washed with saturated sodium bicarbonate (2×50 mL) and brine (2×50 mL). Organic layers were collected and dried over anhydrous magnesium sulphate. Solvents were removed in vacuo to afford the product as a colourless oil (0.062 g, 70% yield). ¹H NMR (CDCl₃, 500 MHz) δ 1.41 (s, 9H), 2.47 (t, 2H, J=5.0 Hz), 2.64 (t, 2H, J=5.0 Hz), 3.29 (s, 2H), 3.43 (q, 2H, J=5.0 Hz), 3.53 (q, 2H, J=5.0 Hz), 3.58-3.61 (m, 8H), 3.65 (s, 3H), 5.14 (s, 1H), 6.46 (s, 1H); ¹³C NMR (CDCl₃, 125 MHz) δ 28.2, 28.5 (CH₃), 29.4, 29.8, 30.9, 39.4, 40.4 (CH₂), 51.9 (CH₃), 67.2, 70.0, 70.2, 70.3, 70.5, 70.53 (CH₂), 156.2, 171.6, 173.6 (C═O). ESI−MS: calculated for C₁₈H₃₄N₂O₈: 406.2; found: 307.1 [M-COO(CH₃)₃+H]⁺.

Methyl 1-amino-13-oxo-3,6,9-trioxa-12-azahexadecan-16-oate (8). Compound 7 (0.062 g, 0.153 mmol) was dissolved in DCM (3 mL) and trifluoroacetic acid (0.5 mL) added dropwise to the reaction mixture. The reaction mixture was allowed to stir overnight at room temperature. The solvent was removed in vacuo and the product was afforded as a colourless oil (0.054 g, 84% yield). The compound was used without further purification. ¹H NMR (CDCl₃, 500 MHz) δ 2.50 (t, 2H, J=5.0 Hz), 3.19 (s, 2H), 3.43 (t, 2H, J=5.0 Hz), 3.59 (t, 2H, J=5.0 Hz), 3.61-3.71 (m, 13H), 3.82 (t, 2H, J=5.0 Hz), 4.15 (bs, 2H), 7.63 (bs, 1H), 8.04 (bs, 3H); ¹³C NMR (CDCl₃, 125 MHz) δ 28.2, 29.0, 30.1, 39.5, 39.8 (CH₂), 52.1 (CH₃), 66.8, 69.5, 69.6, 70.0, 70.1, 70.3 (CH₂), 172.7, 175.2 (C═O); ¹⁹F NMR (CDCl₃, 470 MHz) δ−75.76 (CF₃). ESI−MS: calculated for C₁₃H₂₇N₂O₆⁺: 307.2; found: 307.2 [M]⁺; calculated for CF₃COO: 112.5; found: 113.0 [M]⁻.

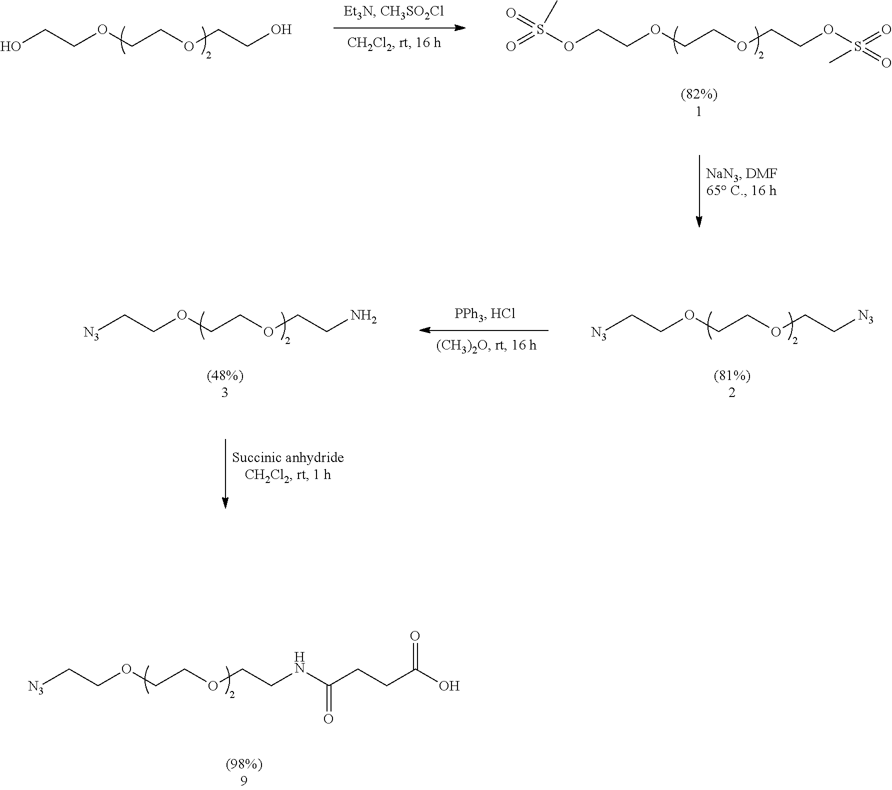

1-Azido-13-oxo-3,6,9-trioxa-12-azahexadecan-16-oic acid (9). Compound 3 (1.45 g, 0.00664 mol) was combined with succinic anhydride (1.33 g, 0.0133 mol) in dry DCM and stirred at room temperature overnight. The reaction mixture was washed with water (2×50 mL) and the aqueous layer back-extracted with DCM to remove residual organic solvents. The aqueous layers were combined and water removed in vacuo. The crude product was purified by column chromatography (silica, neat DCM ˜3% v/v methanol in DCM with 0.5% v/v glacial acetic acid) affording compound 9 as a white solid (2.08 g, 98% yield). ¹H NMR (DMSO-d₆, 500 MHz) δ 2.31 (t, 2H, J=10.0 Hz), 2.40 (t, 2H, J=10.0 Hz), 3.175 (q, 2H, J=5.0 Hz), 3.39 (t, 2H, J=5.0 Hz), 3.52-3.57 (m, 8H), 3.59-3.61 (m, 2H); ¹³C NMR (DMSO-d₆, 125 MHz) δ 28.8, 29.1, 29.9, 38.6, 50.0, 69.1, 69.2, 69.6, 69.7, 69.75, 69.78 (CH₂), 173.6, 173.8 (C═O). MALDI-TOF: calculated for C₁₂H₂₂N₄O₆: 318.15; found: 319.14 [M+H]⁺, 341.12 [M+Na]*.

### Example 2—MeCOSAR Modifications

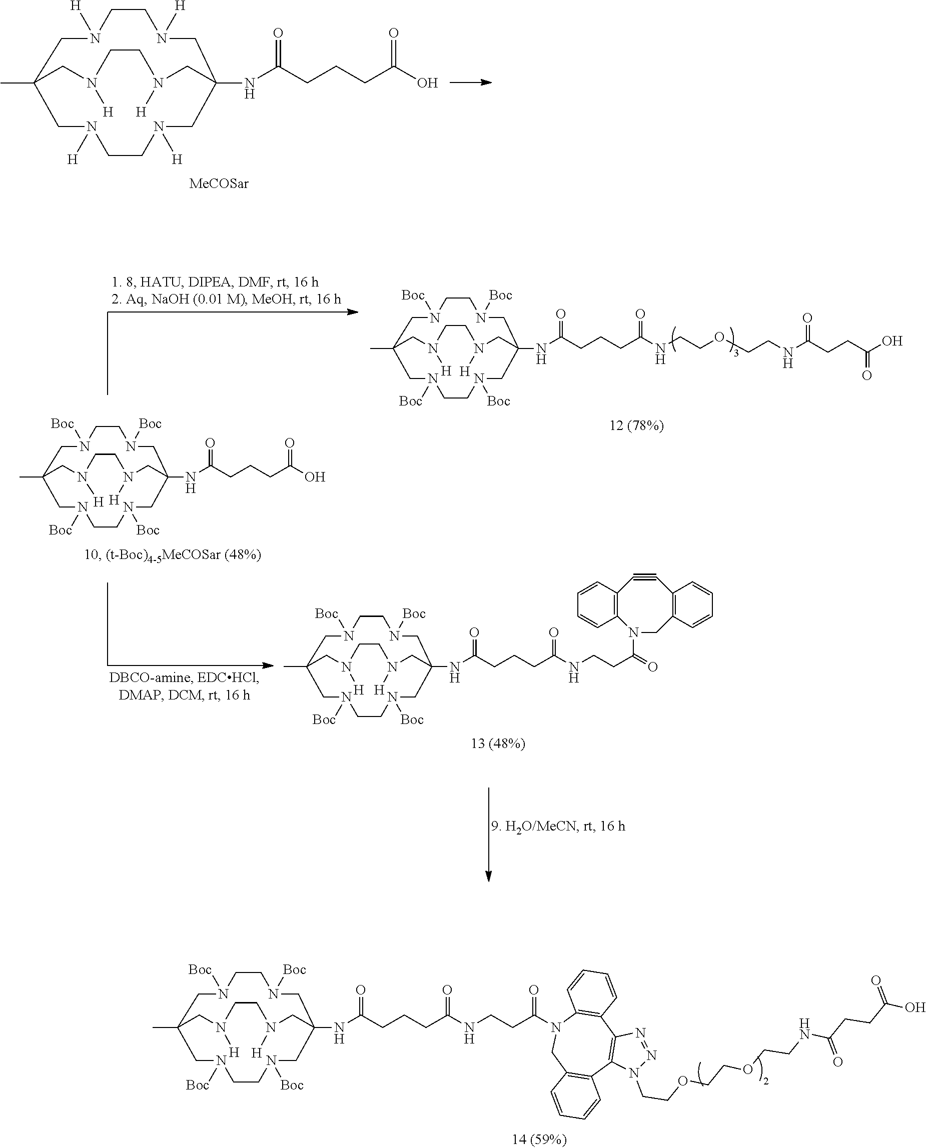

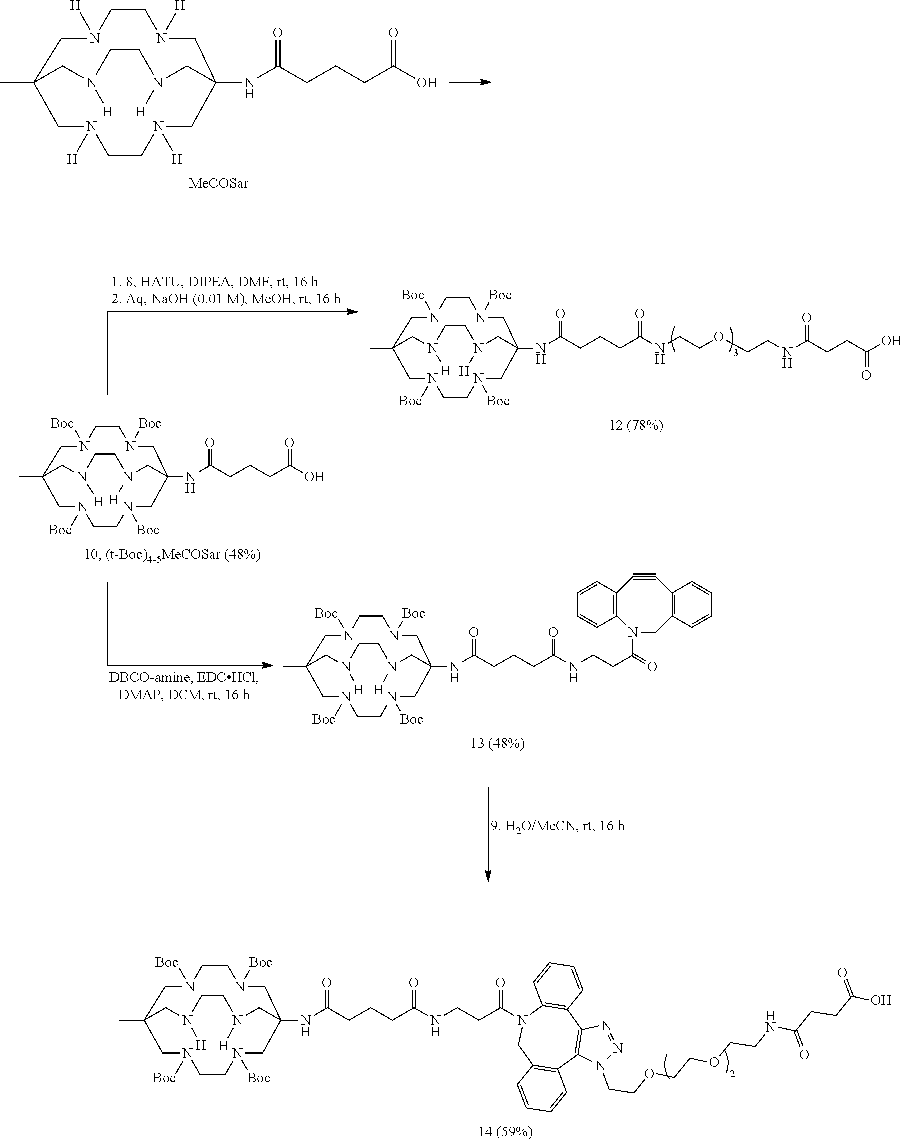

(t-Boc)₄₋₅MeCOSar-PEG3-methy ester (11) (tBoc)₄₋₅MeCOSar (3.6 mg, 4.35 mol), HATU (1.9 mg, 0.00481 mmol) and DIPEA (0.732 mg, 5.66 mol) were dissolved in dry DMF (0.3 mL), and compound 8 (1.9 mg, 4.35 μmol) added. The solution was purged with a gentle flow of nitrogen and allowed to stir at room temperature overnight. The residue was diluted with DCM and washed with aqueous ammonium chloride (2×5 mL), water (1×5 mL) and brine (2×5 mL). Organic layers were collected, dried over anhydrous magnesium sulphate, and removed in vacuo affording the product as a white solid (4.0 mg, 82% yield). ESI−MS: calculated for C₅₃H₉₇N₉O₁₆: 1115.71; found 1116.73 [M+H]+, 1138.71 [M+Na]⁺, calculated for C₅₈H₁₀₅N₉O₁₈: 1215.76; found: 1216.77 [M+H]⁺, 1238.76 [M+Na]⁺.

(t-Boc)₄₋₅MeCOSar-PEG3 (12). Compound 11 was dissolved in methanol (0.45 mL) and aq. NaOH (0.01M, 0.3 mL) added to the reaction mixture. Reaction was stirred at room temperature overnight. The resulting mixture was diluted with DCM and washed with aqueous ammonium chloride (2×5 mL), water (2×5 mL) and brine (2×5 mL). The organic layers were collected, dried over anhydrous magnesium sulphate, and removed under reduced pressure. The product was afforded as a white powder (3.0 mg, 7600 yield). ESI−MS: calculated for C₅₂H₉₅N₉O₁₆: 1101.69; found: 1102.73 [M+H]⁺, 1124.77 [M+Na]⁺, calculated for C₅₇H₁₀₃N₉O₁₈: 1201.74; found: 1224.77 [M+Na]⁺.

(t-Boc)₄₋₅MeCOSar-DBCO (13). Dibenzocyclooctyne-amine (1.67 mg, 0.00604 mmol) was dissolved in DCM (0.4 mL). N-(3-dimethylaminopropyl)-N′-ethylcarbodiimide hydrochloride (1.16 mg, 6.04 μmol), 4-dimethylaminopyridine (0.185 mg, 1.51 μmol) and (t-Boc)₄₋₅MeCOSar (5 mg, 6.04 μmol) were added to the reaction mixture. The reaction mixture was purged with a gentle flow of nitrogen and allowed to stir at room temperature overnight. The resulting mixture was washed with aqueous ammonium chloride (2×5 mL), water (1×5 mL) and brine (1×5 mL). The organic layers were collected, dried over anhydrous magnesium sulphate and removed under reduced pressure. The product was afforded as a white solid (3.0 mg, 46% yield). ESI−MS: calculated for C₅₈H₈₇N₉O₁₁: 1085.65; found: 1086.66 [M+H]⁺, calculated for C₆₃H₉₅N₉O₁₃: 1186.70; found: 1186.70 [M+H]⁺.

(t-Boc)₄₋₅MeCOSar-DBCO-triazole-PEG3 (14). The SPAAC reaction was performed by dissolving compounds 9 (1.5 mg, 4.72 μmol) and 13 (6.0 mg, 5.53 μmol) in a mixture of water and acetonitrile (1:1, 0.5 mL). The reaction mixture was stirred overnight at room temperature. The crude mixture was lyophilized affording the product as a white powder (3.9 mg, 59% yield). ESI−MS: calculated for C₇₀H₁₀₉N₁₃O₁₇: 1403.81; found: 1386.80 [M+H−H₂O]⁺, 1404.81 [M+H]⁺.

### Example 4—Peptide Conjugation

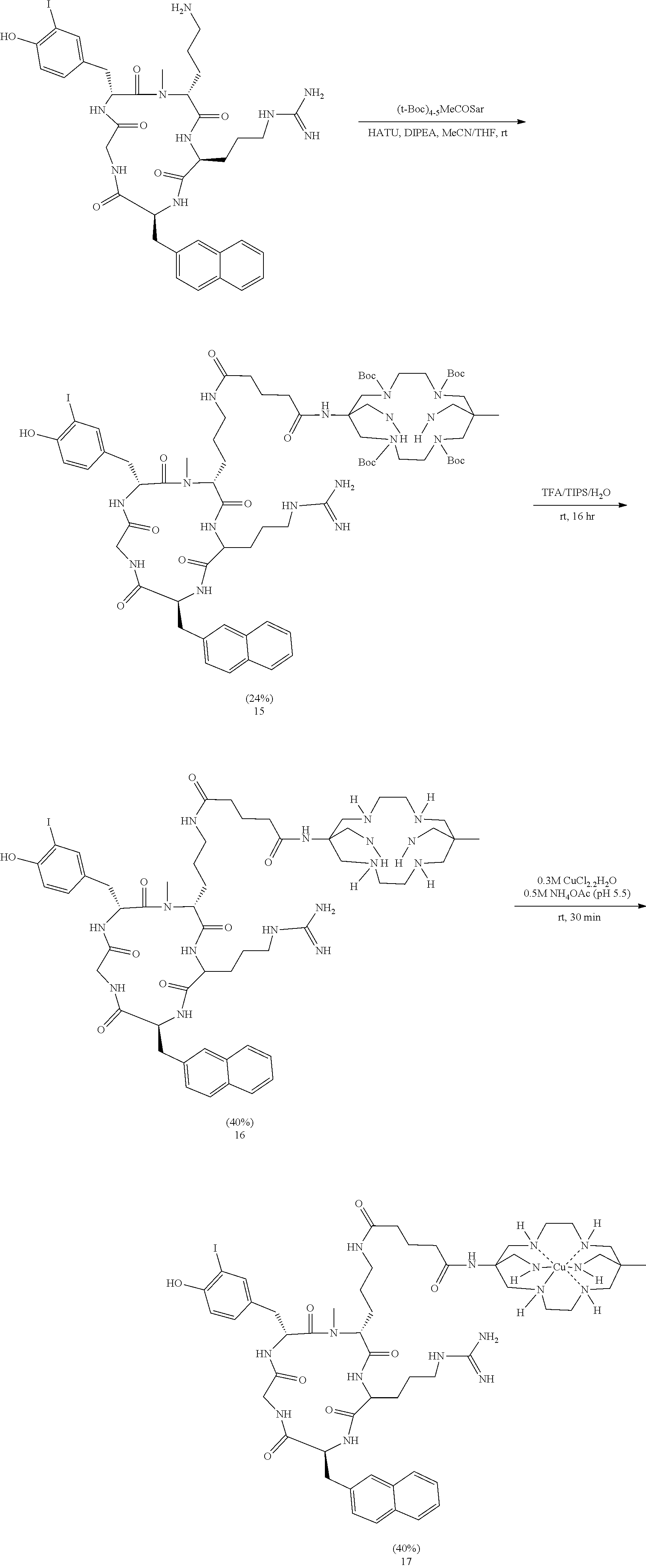

(t-Boc)₄₋₅MeCOSar-(cyclo(D-3-iodo-Tyr¹-[NMe]-D-Orn²-Arg³-2-Nal⁴-Gly⁵) (15). The peptide, (cyclo(D-3-iodo-Tyr¹-[NMe]-D-Orn²-Arg³-2-Nal⁴-Gly⁵) (3.2 mg, 3.87 μmol) was dissolved in acetonitrile and THE (1:1, 200 μL), and added to a mixture of (t-Boc)₄₋₅MeCOSar (3.2 mg, 3.87 μmol) and HATU (2.94 mg, 7.74 μmol). DIPEA (2 μL) was added to the reaction mixture, and stirred at room temperature overnight. The solvents were removed under a gentle N₂ flow. The crude mixture was purified by semi-preparative HPLC. The fractions were collected and lyophilized to give compound 15 as a white solid (1.5 mg, 24% yield). HPLC: t_{R}=11.2 min. ESI−MS: calculated for C₆H₁₁₇IN₁₆O₁₆: 1636.79; found: 819.41 [M+2H]²+, 1637.80 [M+H]⁺.

MeCOSar-(cyclo(D-3-iodo-Tyr¹-[NMe]-D-Orn²-Arg³-2-Na⁴-Gly⁵) (16). t-Boc group cleavage was carried out by adding a solution (100 μL) containing triisopropylsilane (10%), water (10%) and trifluoroacetic acid (80%) to compound 15 (1.5 mg, 0.916 μmol). The reaction mixture was allowed to stir at room temperature overnight. The crude mixture was purified by semi-preparative HPLC. The fractions were collected and lyophilized to afford compound 16 as a white solid (0.453 mg, 40% yield). HPLC: t_{R}=5.33 min. ESI−MS: calculated for C₅₆H₈₅1N₁₆O₈: 1236.58; found: 619.30 [M+2H]²+, 413.20 [M+3H]3.

[^{nat}Cu]MeCOSar-(cyclo(D-3-iodo-Tyr¹-[NMe]-D-Orn²-Arg³-2-Na⁴-Gly) (17). The non-radioactive reference compound was prepared by mixing an aqueous solution of CuCl₂.2H₂O (0.3 M, 1.5 μL) with a solution of compound 16 (0.5 mg, 0.385 μmol) in ammonium acetate buffer (0.5 M, pH 5.5, 150 μL). The reaction was carried out at room temperature for 30 minutes. The crude mixture was purified by semi-preparative HPLC and the fractions collected and lyophilized to give complex 17 as a white solid (0.2 mg, 40% yield). HPLC: t_{R}=5.59 min. ESI−MS: calculated for C₅₆H₈₅CuIN₁₆O₈: 1299.51; found: 649.75 [M]²⁺, 433.50 [M]³⁺.

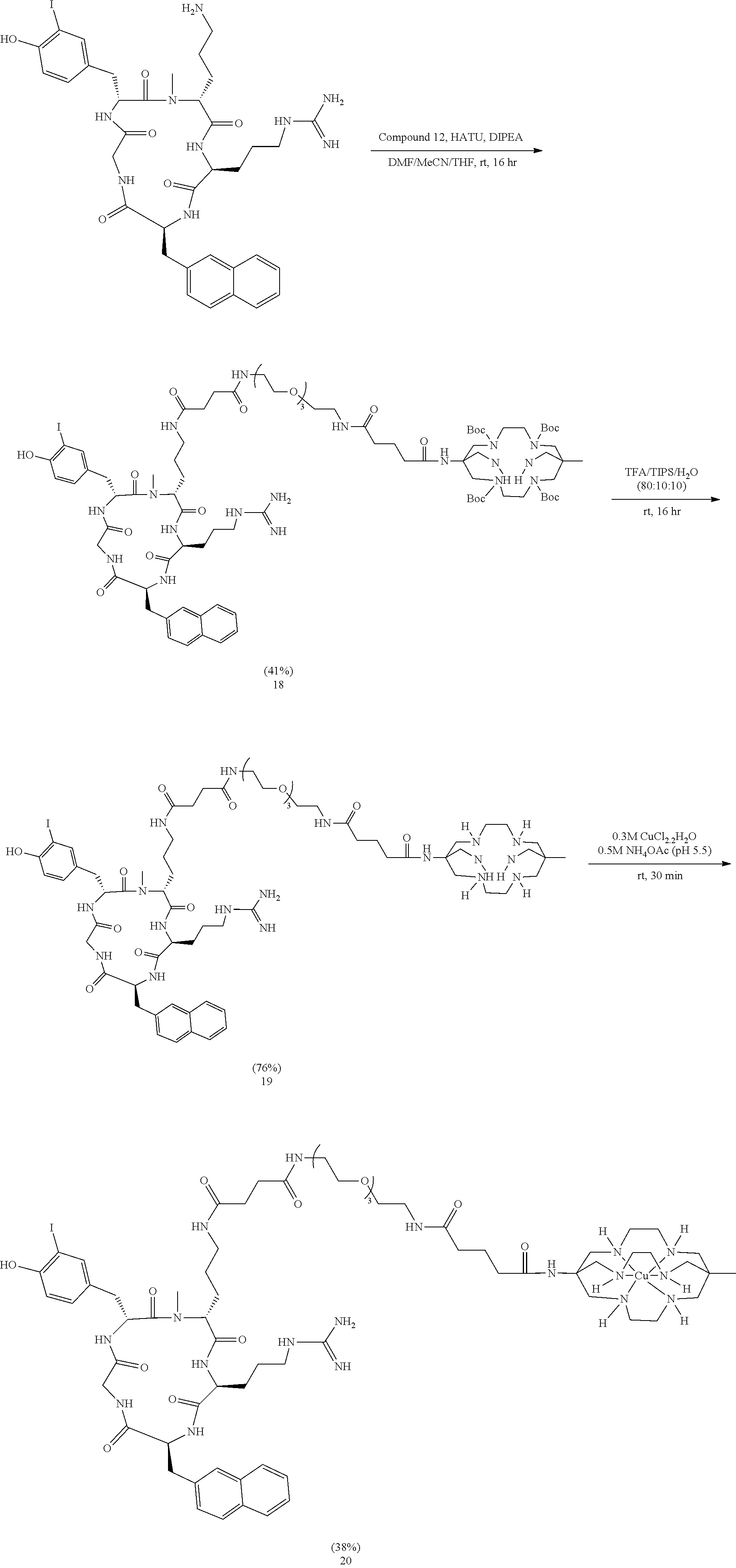

(t-Boc)₄₋₅MeCOSar-PEG3-(cyclo(D-3-iodo-Tyr¹-[NMe]-D-Orn²-Arg³-2-Nal⁴-Gly⁵) (18). Compound 12 (0.7 mg, 0.635 μmol) was dissolved in DMF (100 μL) and added to a solution containing the peptide (0.526 mg, 0.635 μmol) and HATU (0.483 mg, 1.27 μmol) in a mixture of acetonitrile and THE (1:1, 100 μL). DIPEA was added to the reaction mixture and stirred at room temperature overnight. The crude reaction mixture was purified by semi-preparative HPLC, affording compound 18 as a white solid (0.5 mg, 41% yield). HPLC: t_{R}=7.99 min. ESI−MS: calculated for C₈₈H₁₃₉IN₁₈O₂₁: 1910.94; found: 956.98 [M+2H]²⁺.

MeCOSar-PEG3-(cyclo(D-3-iodo-Tyr¹-[NMe]-D-Orn²-Arg³-2-Nal⁴-Gly⁵) (19). t-Boc group cleavage was carried out by adding a 100 μL solution containing triisopropylsilane (10%), water (10%) and trifluoroacetic acid (80%) to compound 18. The reaction mixture was allowed to stir at room temperature overnight. The crude mixture was diluted with water and purified by semi-preparative HPLC. The fractions were collected and lyophilized to afford compound 19 as a white solid (0.3 mg, 76% yield). HPLC: t_{R}=5.33 min. ESI−MS: calculated for C₆₈H₁₀₇1N₁₈O₁₃: 1510.73; found: 756.38 [M+2H]²+, 504.59 [M+3H]³⁺.

[^{nat}Cu]MeCOSar-PEG3-(cyclo(D-3-iodo-Tyr-[NMe]-D-Orn²-Arg³-2-Na⁴-Gly⁵) (20). The non-radioactive reference compound was prepared by mixing an aqueous solution of CuCl₂.2H₂O (0.3 M, 1.3 μL) with a solution of compound 19 (0.5 mg, 0.331 μmol) in ammonium acetate buffer (0.5 M, pH 5.5, 100 μL). The reaction was carried out at room temperature for 30 minutes. The crude mixture was purified by semi-preparative HPLC and the fractions collected and lyophilized to give complex 17 as a white solid (0.2 mg, 38% yield). HPLC: t_{R}=5.55 min. ESI−MS: calculated for C₆₈H₁₀₇CuIN₁₈O₁₃: 1573.66; found: 786.83 [M]²⁺, 524.88 [M]³⁺.

### Example 5—Synthesis NHS-Ester of (t-Boc)₄₋₅ BisCOSar

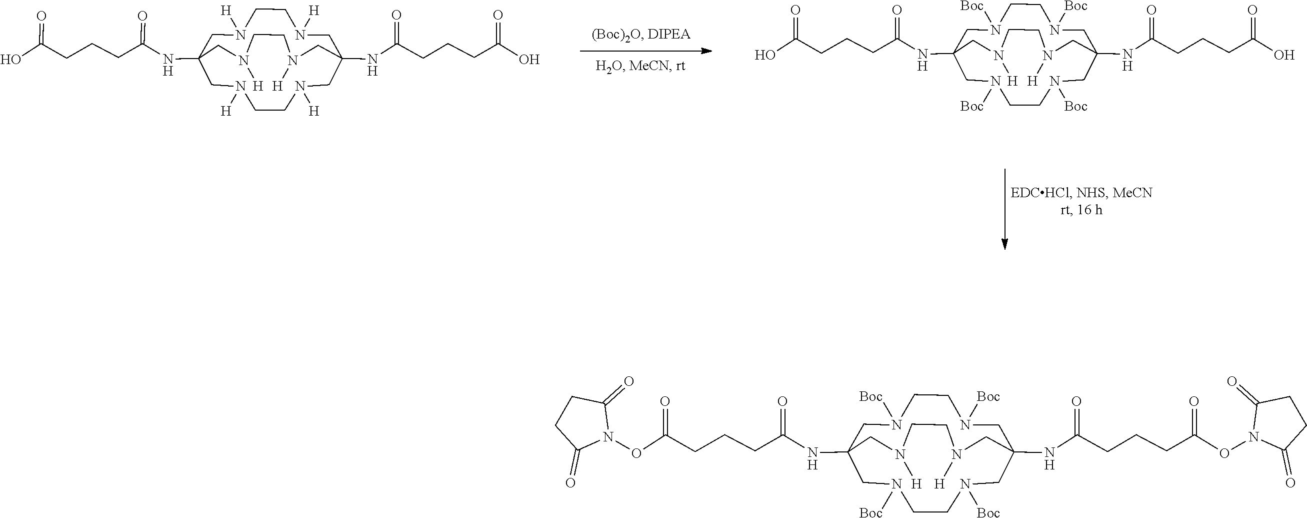 [SMILES: O=C(O)CCCC(=O)NC12CNCCNCC(NC(=O)CCCC(=O)O)(CNCCNC1)CNCCNC2 ; CC(C)(C)OC(=O)N1CCN(C(=O)OC(C)(C)C)CC2(NC(=O)CCCC(=O)O)CNCCNCC(NC(=O)CCCC(=O)O)(C1)CN(C(=O)OC(C)(C)C)CCN(C(=O)OC(C)(C)C)C2 ; CC(C)(C)OC(=O)N1CCN(C(=O)OC(C)(C)C)CC2(NC(=O)CCCC(=O)ON3C(=O)CCC3=O)CNCCNCC(NC(=O)CCCC(=O)ON3C(=O)CCC3=O)(C1)CN(C(=O)OC(C)(C)C)CCN(C(=O)OC(C)(C)C)C2]

### Example 6—Radiolabelling with Copper 64

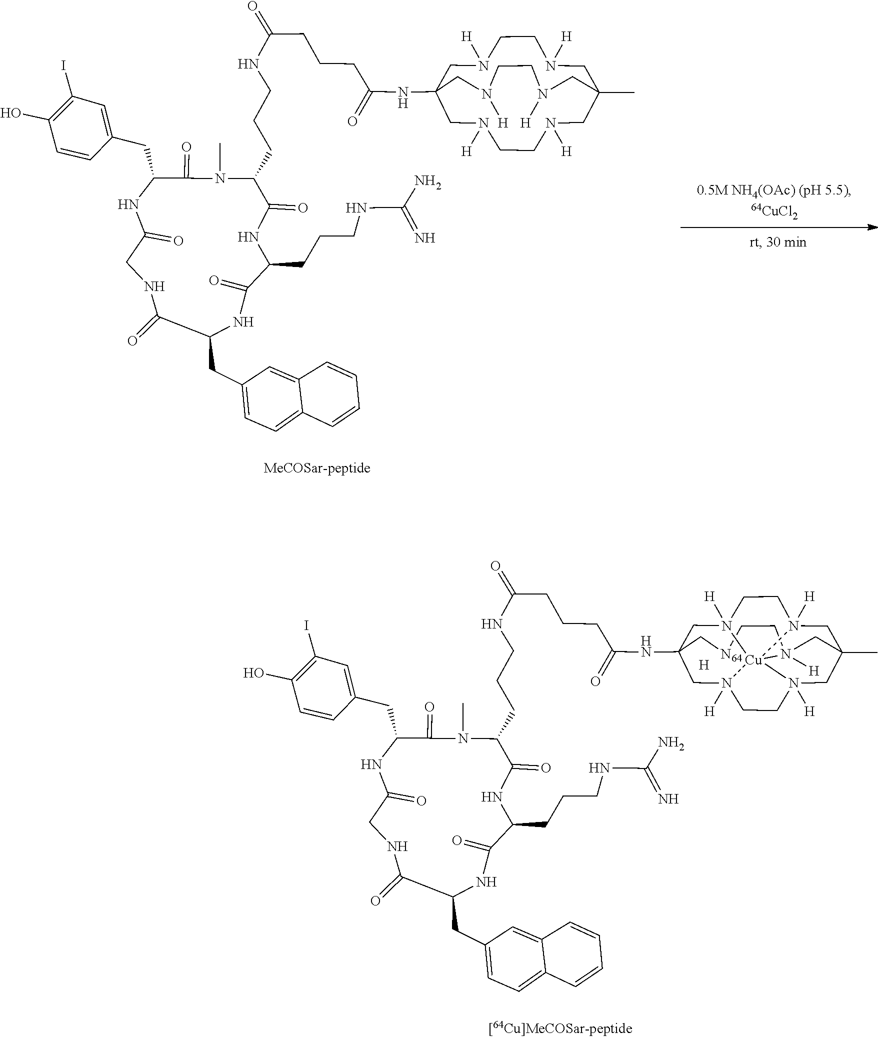

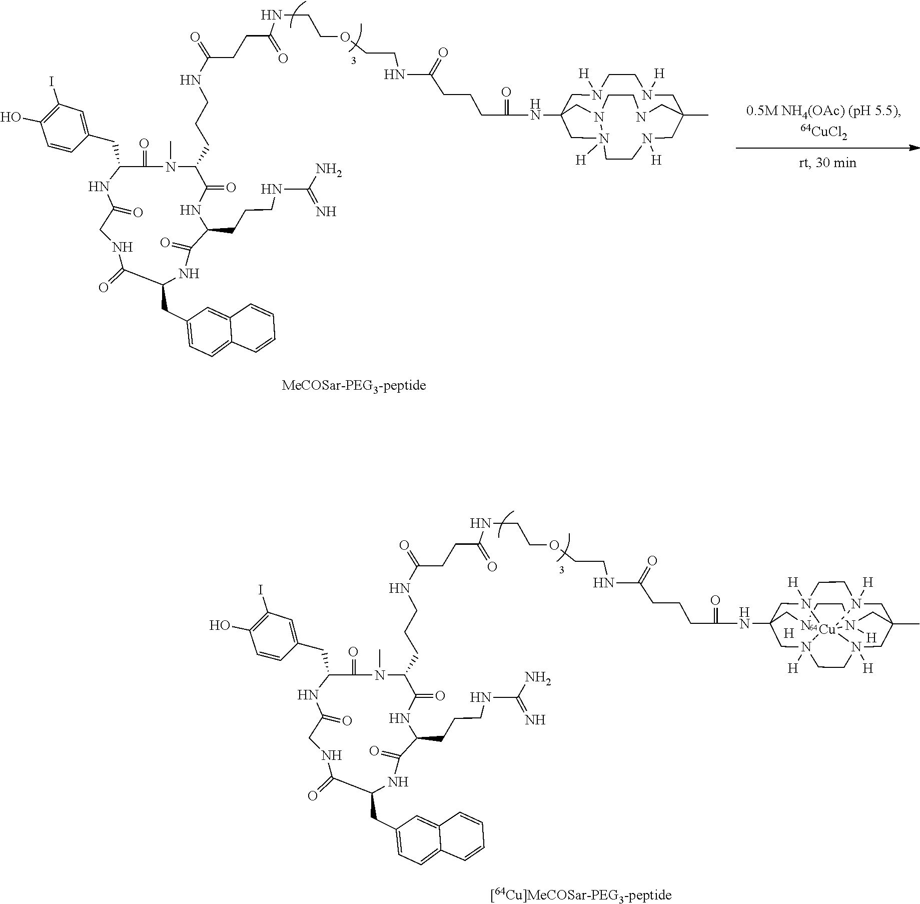

### Example 7—Radiolabelled Peptides for Animal Experiments

Exp 3—MeCOSar-Peptide; Conjugated excess 5000; Prepared dose (MBq) 50; Moles Conjugate 2.74E-08; Moles Cu 5.48E-12; RCP % by iTLC 99%; RCY (%) 96%.

Exp 4—MeCOSar-PEG₃-Peptide; Conjugated excess 1000; Prepared dose (MBq) 20; Moles Conjugate 2.19E-08; Moles Cu 2.19E-12; RCP % by iTLC 99.8%; RCY (%) 96%.

### Example 8 One Pot Synthesis for Ciscosar-Peptide Conjugate

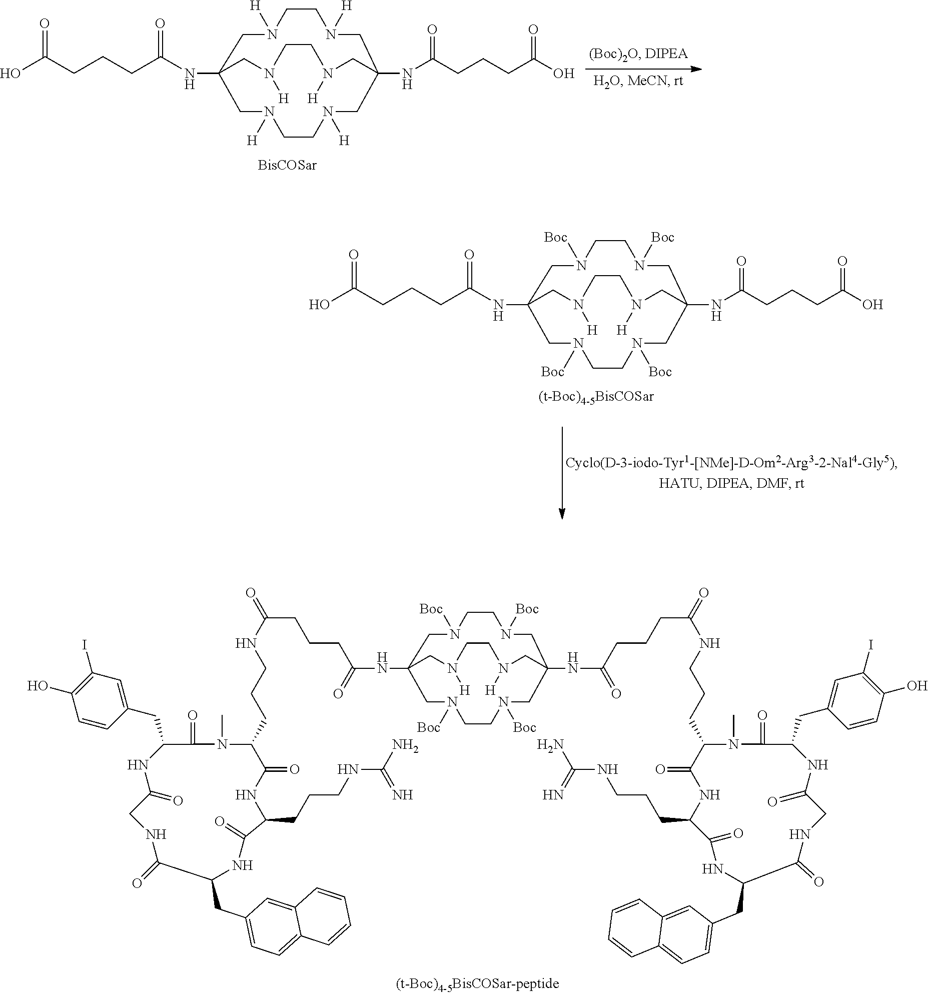

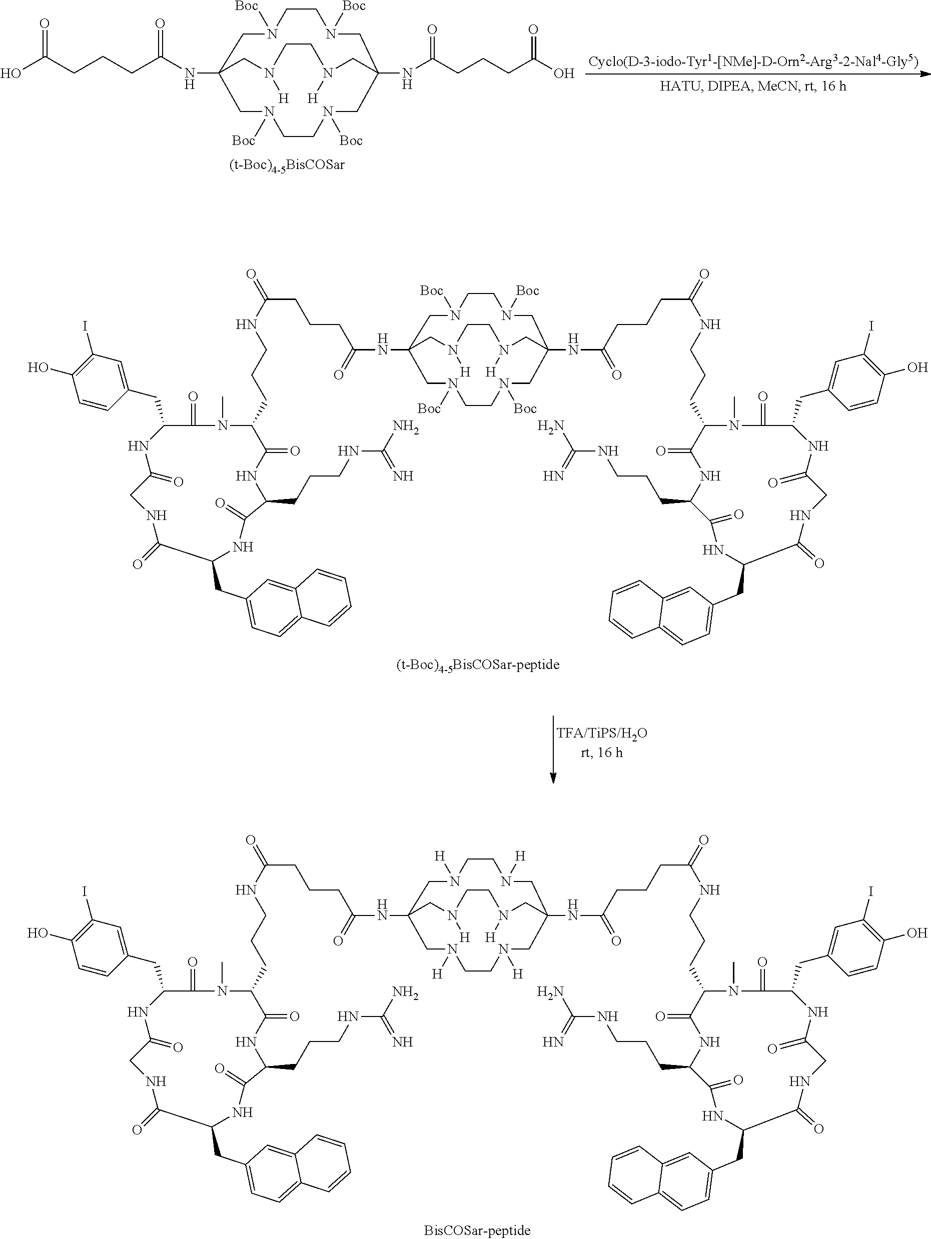

Characterization Data: (t-Boc)₄_BisCOSar-Peptide

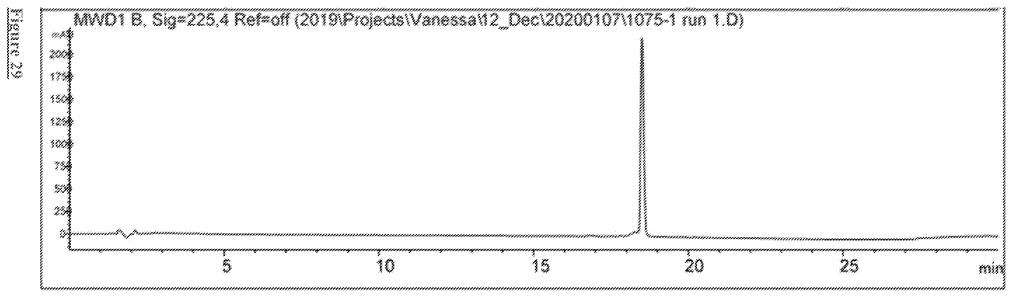

HPLC trace is depicted in FIG. 29

HPLC method: Agilent Eclipse Plus C18 5 μm (4.6×150 mm) column; (A) 0.1% TFA in H₂O, (B) MeCN; gradient method=5-95% (B) in 22 min; flow rate=1 mL/min; λ=225 nm. t_{R}=18.2 min.

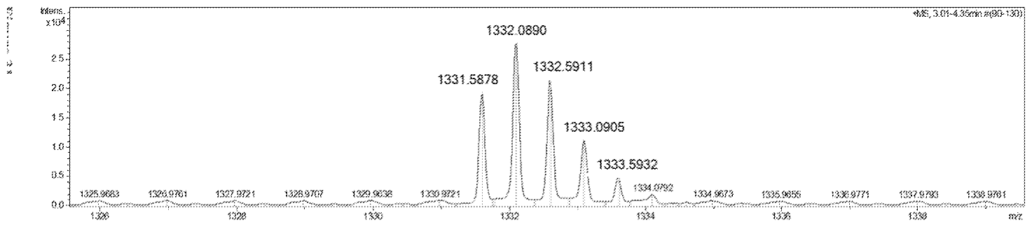

ESI−MS spectrum is depicted in FIG. 31

ESI−MS: calculated for C₁₂₁H₁₇₄I₂N₂₆O₂₆: 2661.12; found: 1332.09 [M+2H]²⁺.

### Example 10 Synthesis of Branched PEG Peptide Analogue

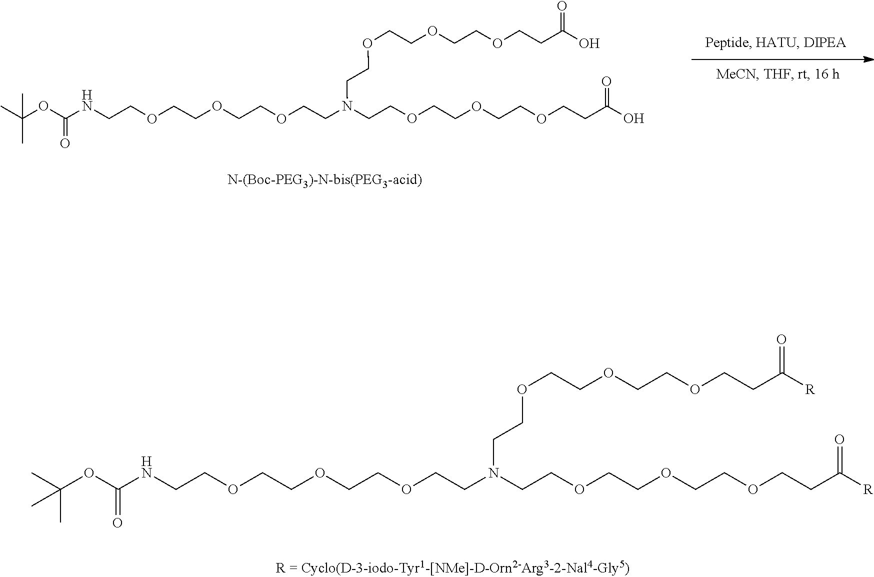

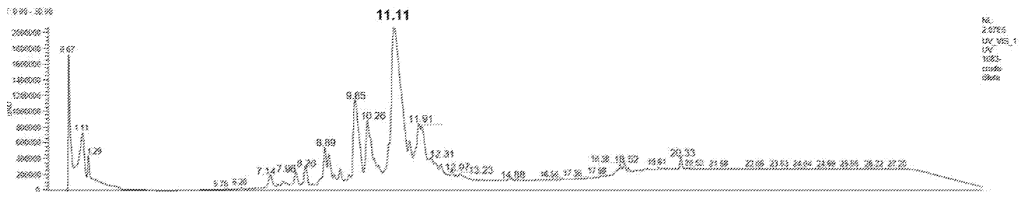

LCMS Trace is Depicted in FIG. 30

LCMS method: Phenomenex Kinetex Agilent Eclipse Plus C18 2.6 μm (50×2.10 mm) column;
(A) 0.2% FA in H₂O, (B) 0.2% FA in 80% MeCN; gradient method=5-100%
(B) in 16.5 min; flow rate=0.2 mL/min; λ=225 nm.

### Example 11 Branched PEG Peptides

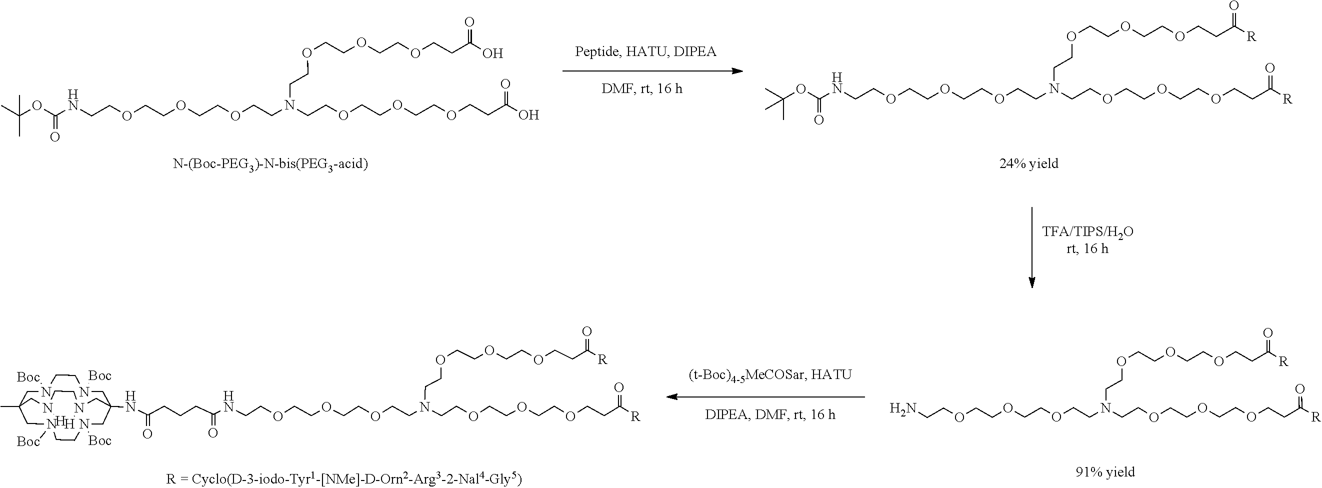

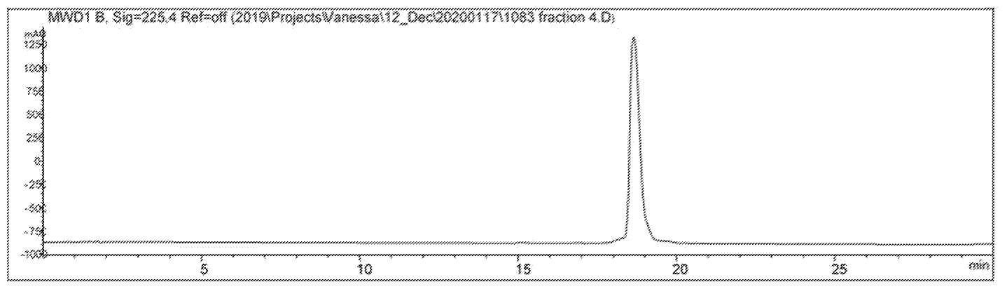

HPLC trace is depicted in FIG. 32.

Analytical HPLC method: Agilent Eclipse Plus C18 5 μm (4.6×150 mm) column; (A) 0.1% TFA in H₂O, (B) MeCN; gradient method=15-50% (B) in 25 min; flow rate=1 mL/min; λ=225 nm. t_{R}=18.6 min.

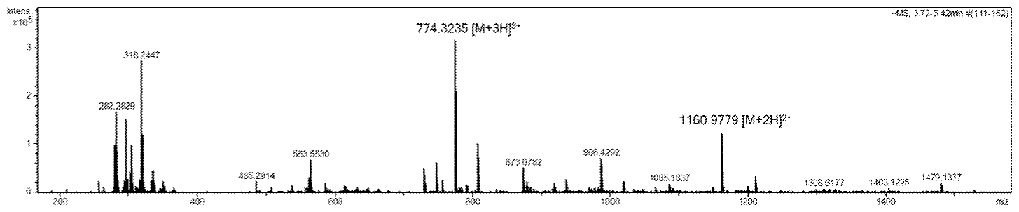

ESI−MS depicted in FIG. 33.

ESI−MS: Calcd. for C₁₀₈ H₁₄₈I₂N₂₀O₂₅: 2318.90; found 1160.98 [M+2H]²⁺, 774.32 [M+3H]³⁺

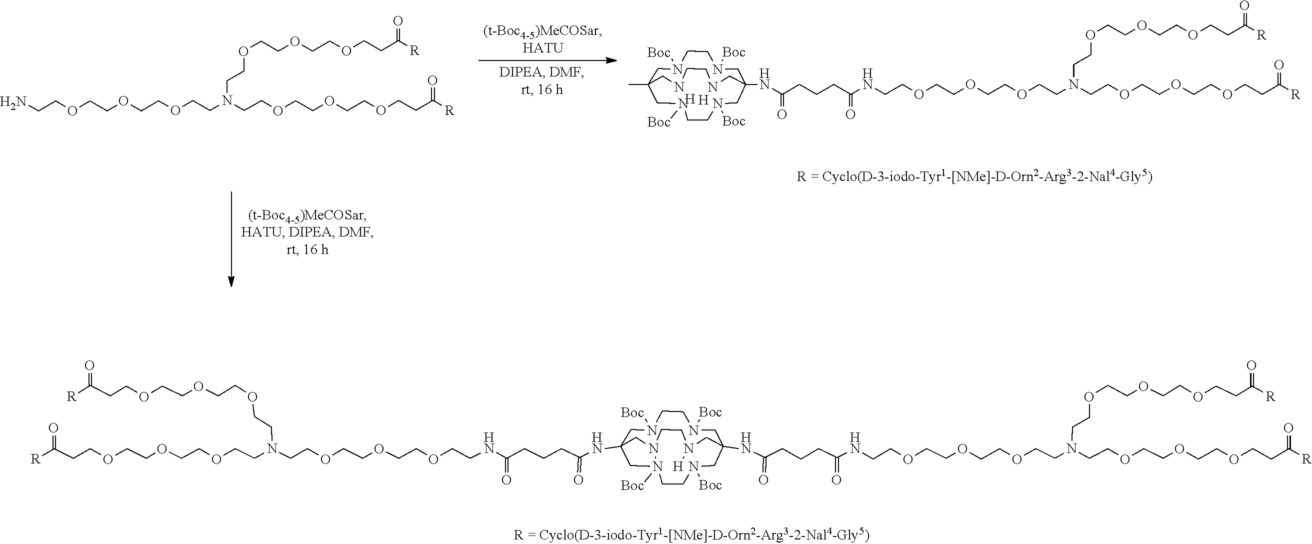

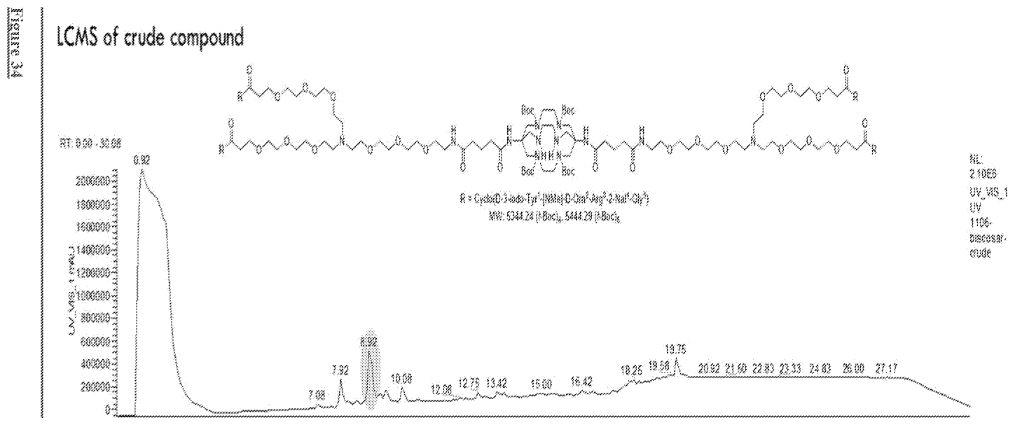

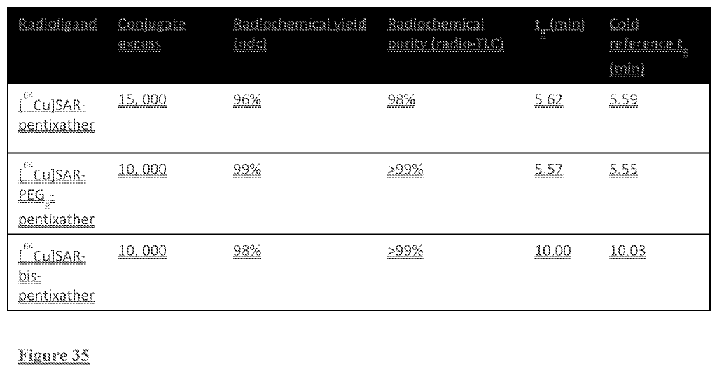

LCMS of the crude bifunctional product is depicted in FIG. 34

### Example 12 Alternative Synthesis for Branched PEG Analogues

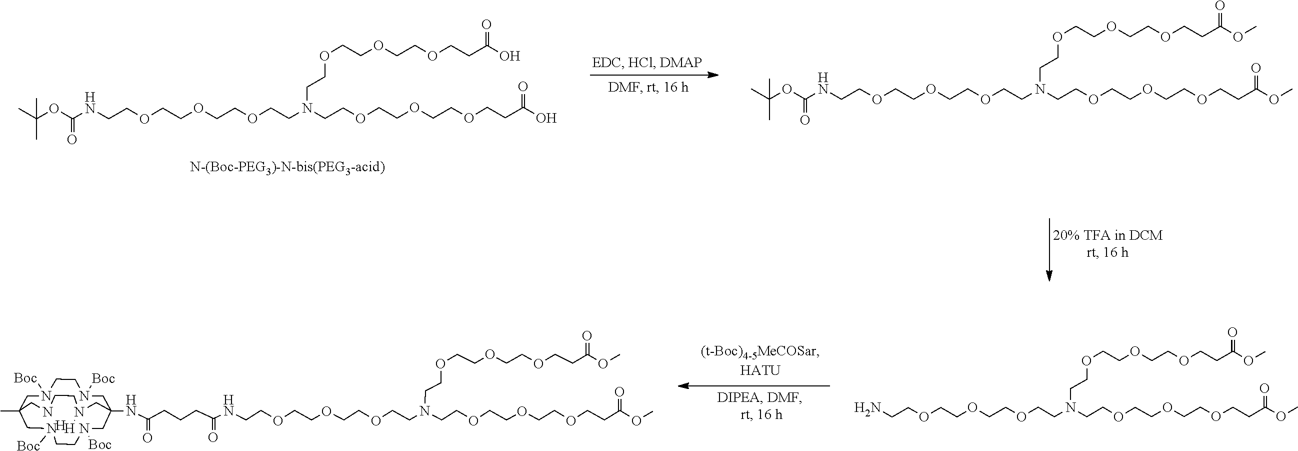

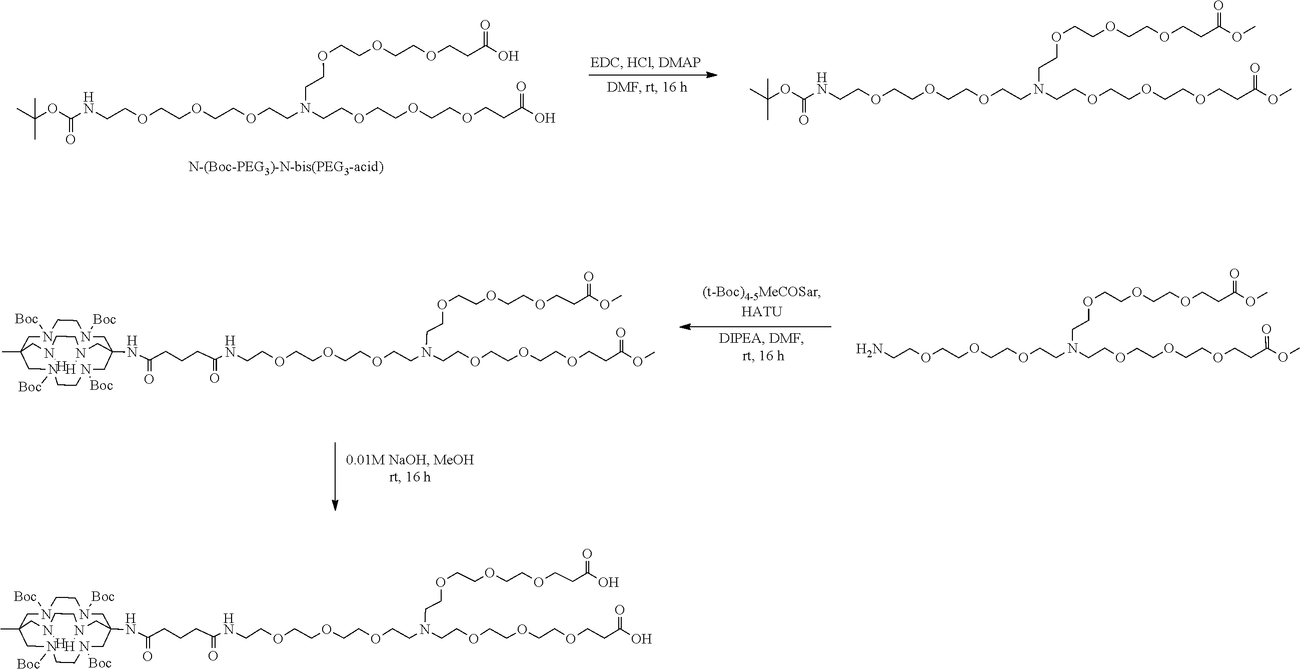

### Example 13 Labelling Compounds

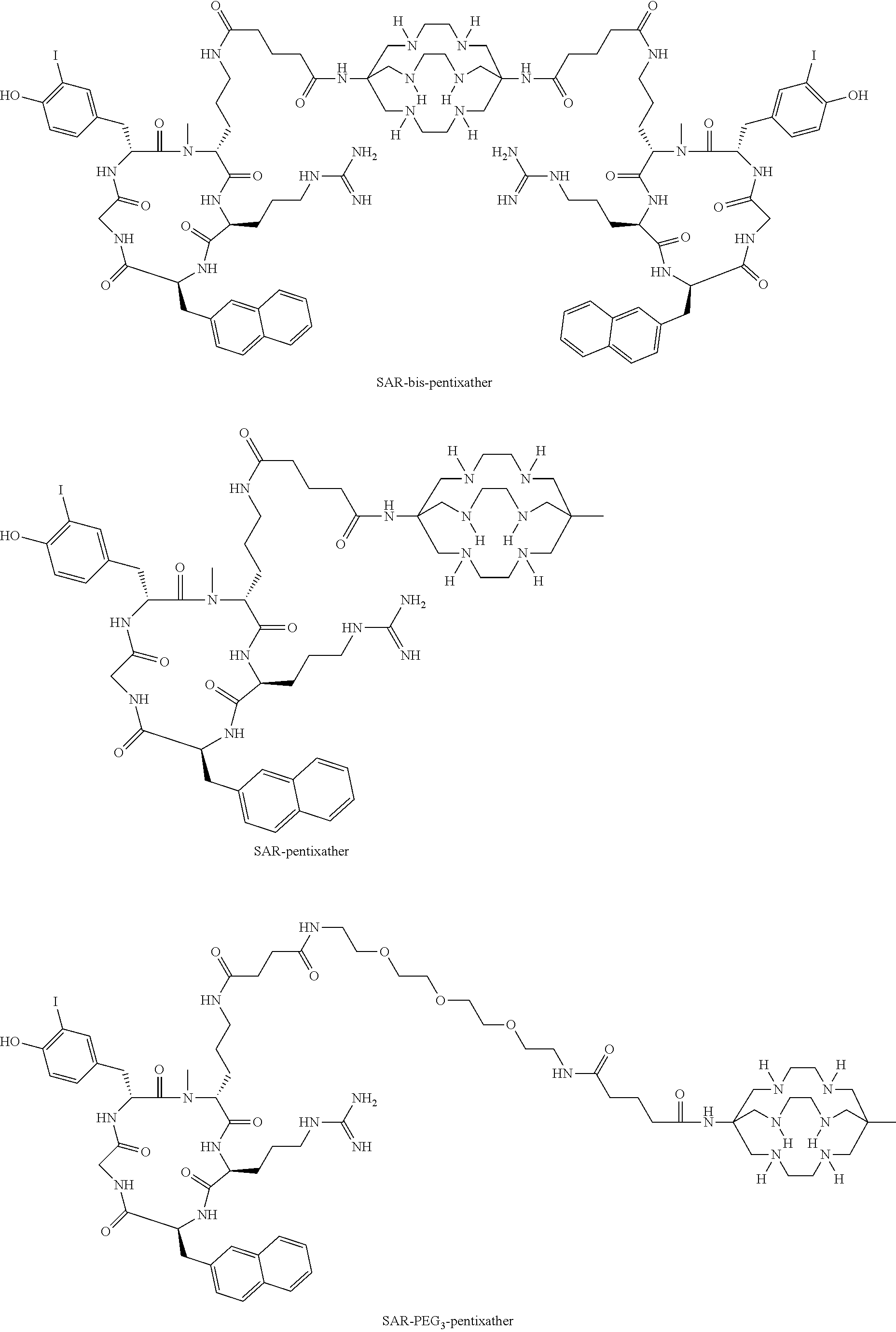

Cu-64 Labelling

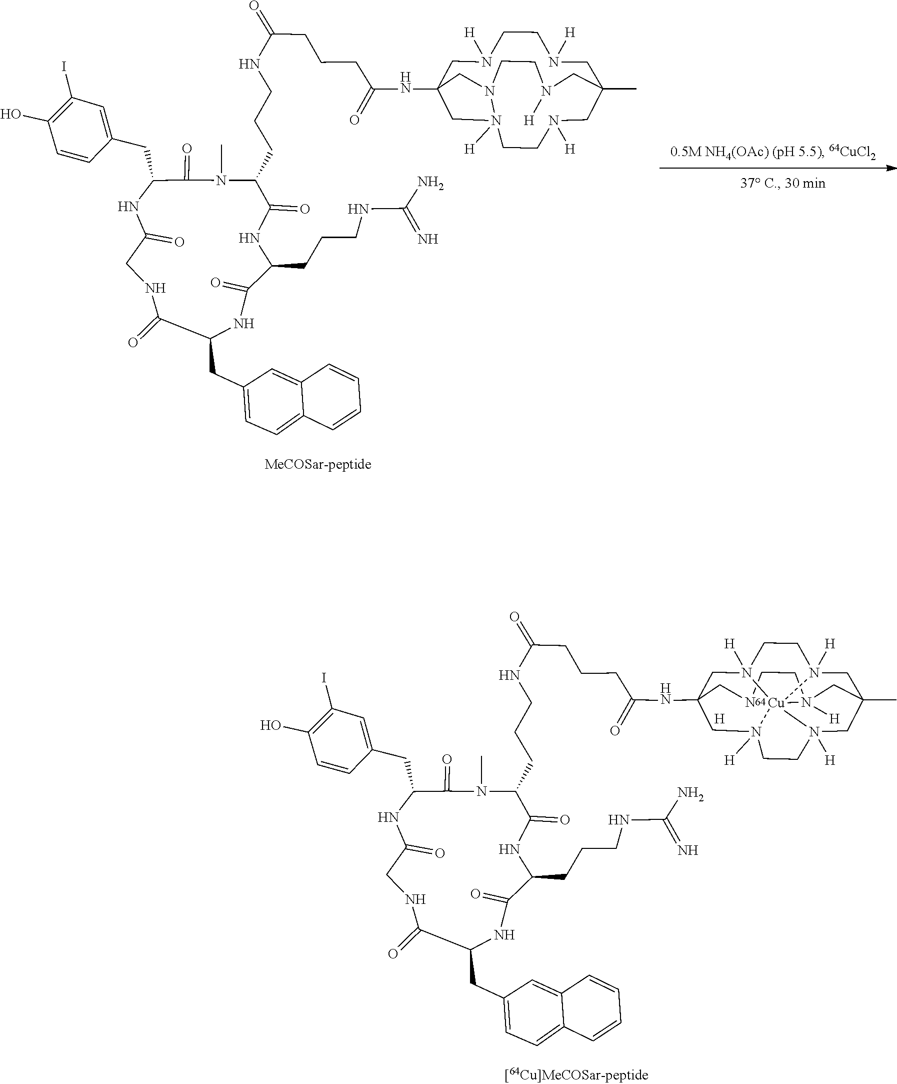

### Example 13—Cell Binding and Internalisation

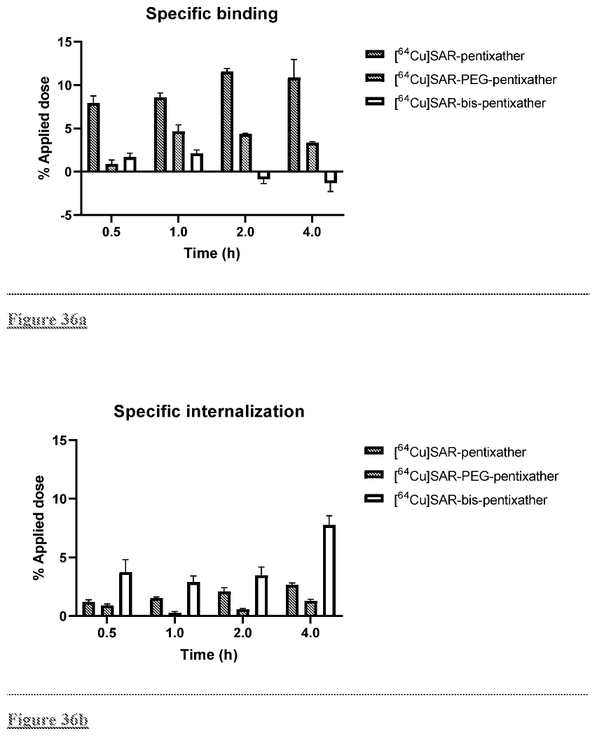

Graphs Depicting the Result are Shown in FIGS. 36a and 36b.

### Example 14 Radiolabel Stability

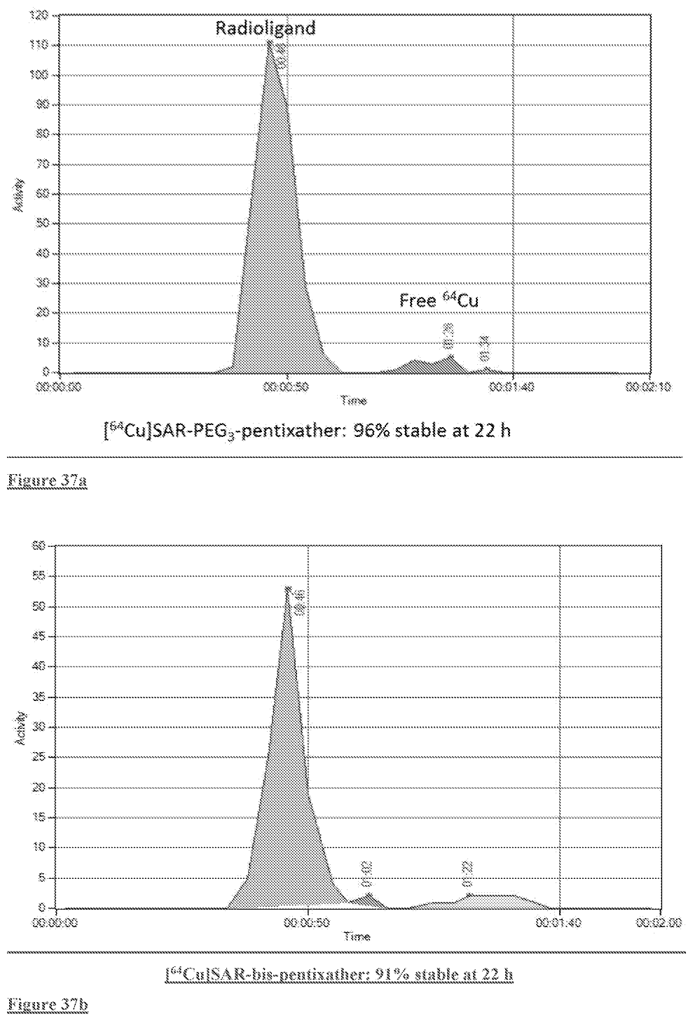

Graphs depicting the results are shown in FIGS. 37a and 37b.

### Example 15—Synthesis of Trimer

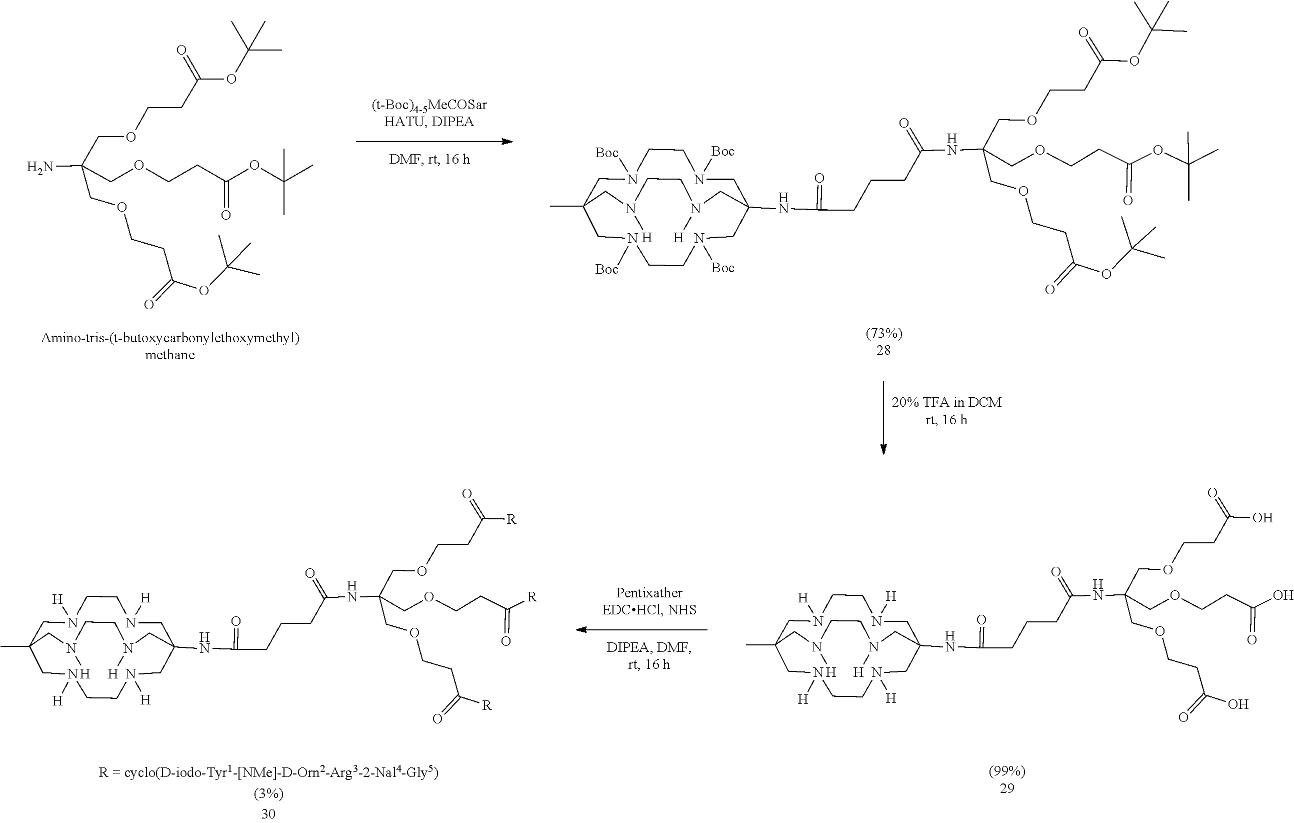

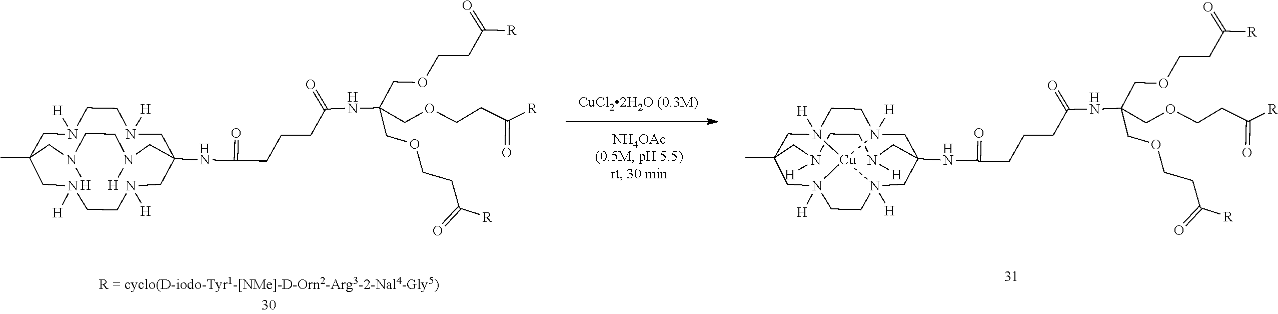

### Example 16—LogD—Lipophility

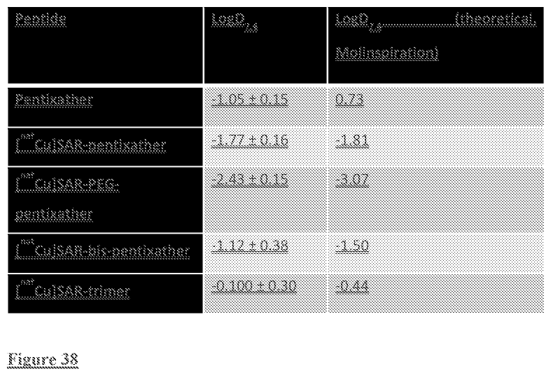

Results are tabulated in FIG. 38.

### Example 17—HSA Trimer Stability

Protocol:
1. 10 MBq of radiotracer incubated with 250 μL of HSA in PBS (50% HAS in PBS)
2. At specific timepoints, 50 μL of HSA is aliquoted out and added to cold MeCN
3. Centrifuged (5 min, 4000 rpm), supernatant removed for HPLC analysis

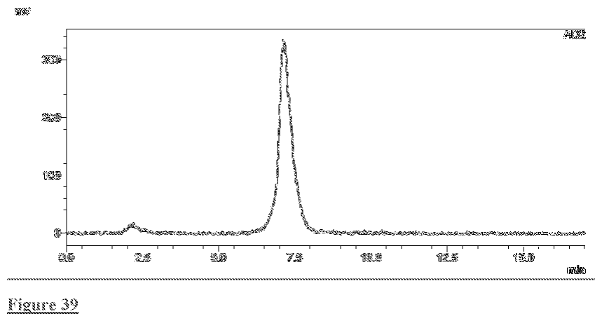

HPLC trace is depicted as FIG. 39.

Analytical HPLC method: Agilent Eclipse Plus C18 5 μm (4.6×150 mm) column; (A) 0.10% TFA in H₂O, (B) MeCN; gradient method=5-95% (B) in 15 min; flow rate=1 mL/min. 24 h, RCP 96%

### Example 17—Binding and Internalization Studies for Trimer

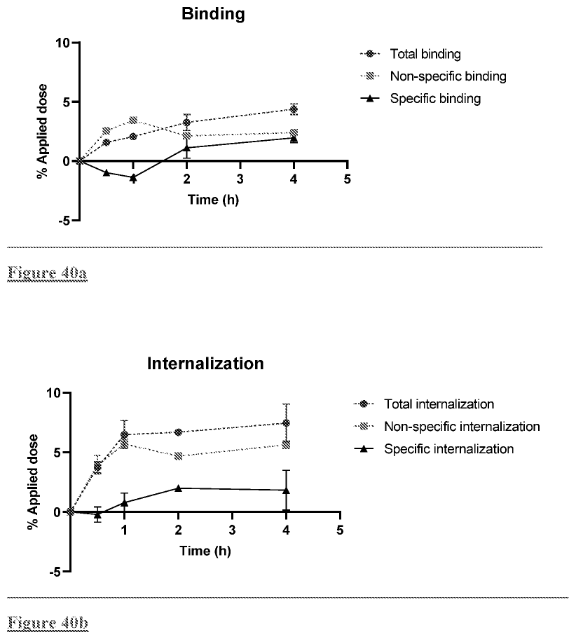

Graphs depicting binding and internalization are shown in FIGS. 40a and 40b.

### Example 18—Binding and Internalization Comparator Studies

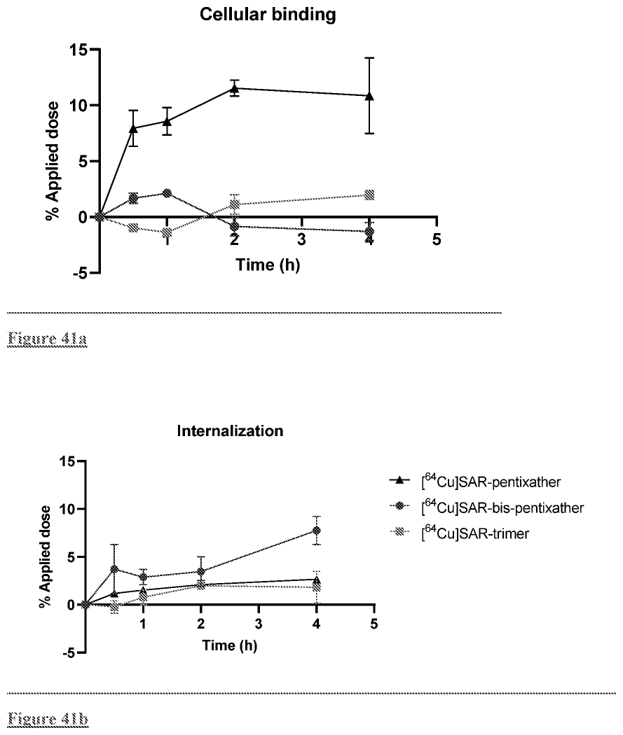

Graphs depicting cellular binding and internalization are shown in FIGS. 41a and 41b.

The claims defining the invention are as follows:

1. A compound of Formula (I) or a salt, complex, isomer, solvate or prodrug thereof: 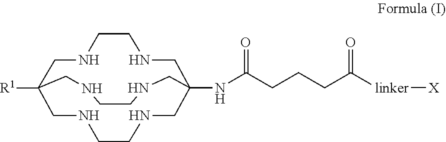
wherein:
R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;
X is 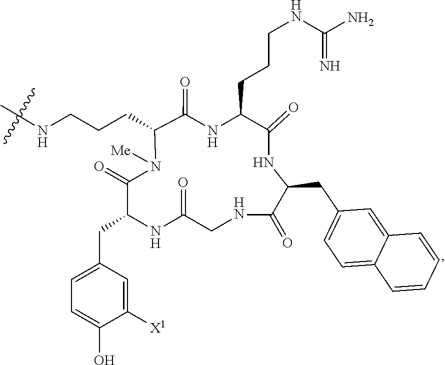
wherein X¹ is H or iodo; and
the linker is absent or selected from: 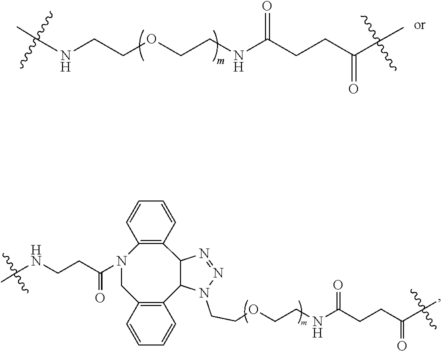
wherein m is independently an integer from 1 to 10.

2. A compound according to claim 1 of Formula (II) or salt, complex, isomer, solvate or prodrug thereof: 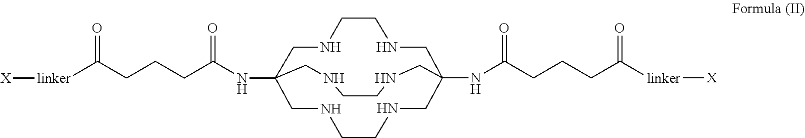
wherein:
X is 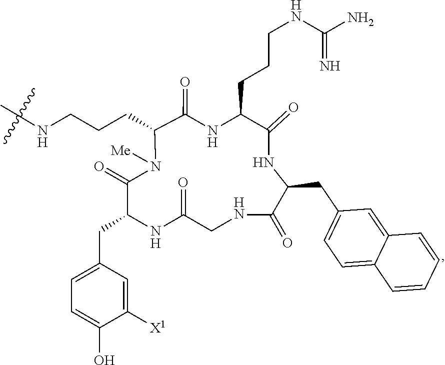
wherein X¹ is H or iodo; and
the linker is absent or selected from: 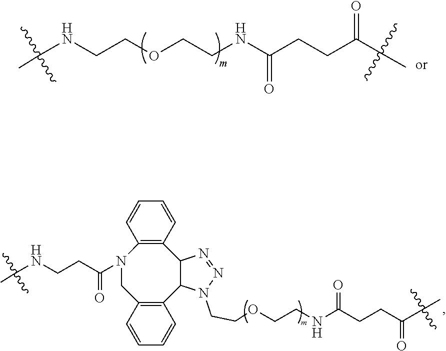
wherein m is independently an integer from 1 to 10.

3. A compound of Formula (III) or a salt, complex, isomer, solvate or prodrug thereof: 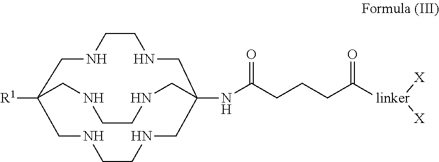
wherein:
R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;
X is 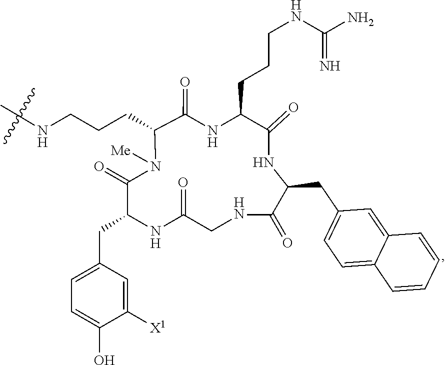
wherein X¹ is H or iodo; and
the linker is selected from: 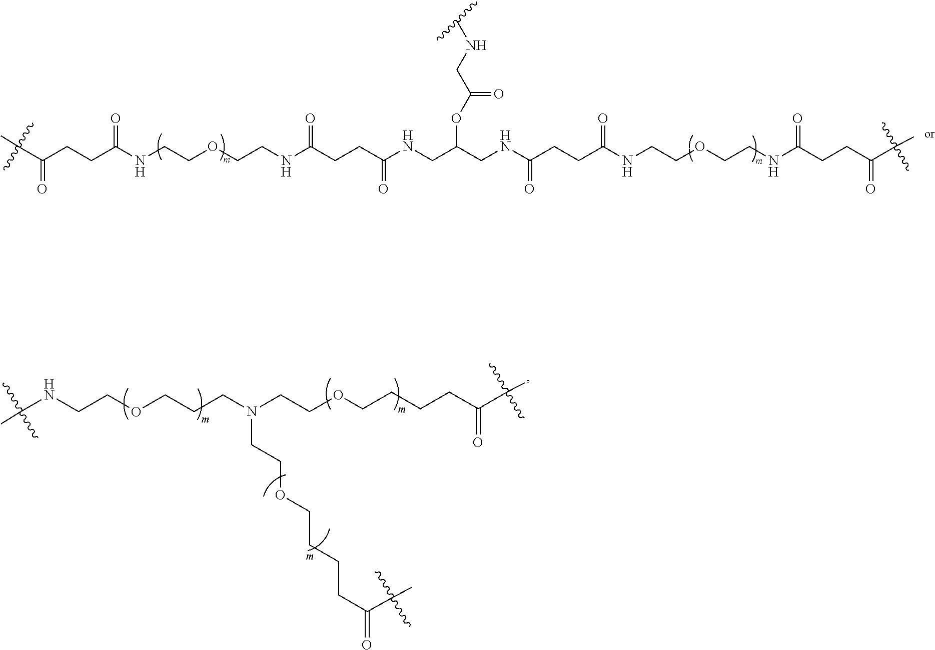
wherein m is independently an integer from 1 to 10.

4. A compound of Formula (IV) or salt, complex, isomer, solvate or prodrug thereof:
wherein: 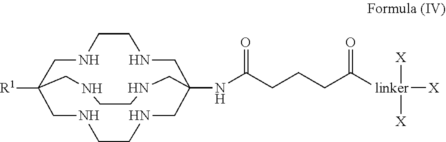
R¹ is selected from the group consisting of H, OH, halogen, cyano, NO₂, optionally substituted C₁-C₁₂ alkyl, optionally substituted alkoxy, optionally substituted acyl, optionally substituted amino, optionally substituted amide and optionally substituted aryl;
X is 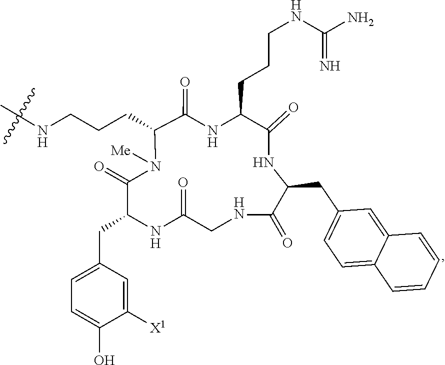
wherein X¹ is H or iodo; and
the linker is selected from: 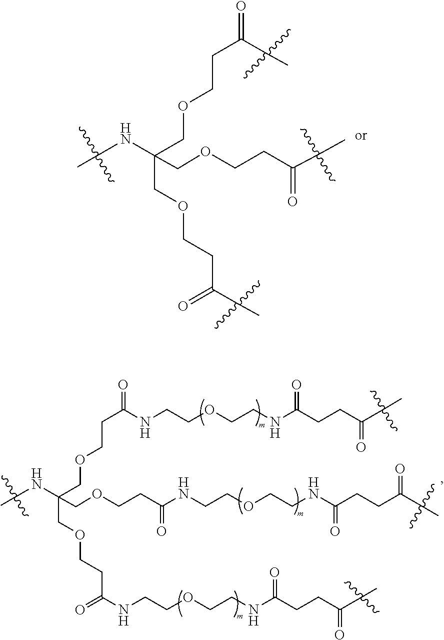
wherein m is an integer from 1 to 10.

5. A compound according to claim 1 or 2, wherein the linker is 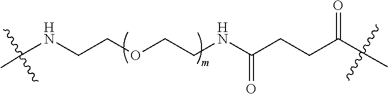
wherein m is defined above.

6. A compound according to anyone of claims 1 to 5, wherein m is selected from 1, 2, or 3.

7. A compound according to claim 6, wherein m is 0.

8. A compound according to claim 7, wherein m is 3.

9. A compound according to anyone of claims 1, and 3-8, wherein R¹ is CH₃.

10. A composition comprising a compound of any one of claims 1 to 9.

11. A compound according to any one of claims 1 to 10 wherein the compound is complexed with a metal.

12. A compound according to claim 11, wherein the metal is copper.

13. A method for radioimaging a subject, the method comprising administering to the subject a compound of claim 11 or 12.

14. A method for treating or preventing a condition in a subject, the method comprising administering to the subject a compound of claim 11 or 12.

15. A method of claim 14, wherein the condition is a cancer or a tumour.

16. Use of a compound of any one of claims 1 to 12 in the manufacture of a medicament for treating or preventing a condition.

17. Use of claim 16, wherein the condition is a cancer or a tumour.
